(19) United States
(12) Patent Application Publication
(10) Pub. No.: US 2015/0158914 A1
(43) Pub. Date: Jun. 11, 2015

(54) PEPTIDES, CONSTRUCTS AND USES THEREFOR

(71) Applicant: Auckland UniServices Limited, Auckland (NZ)

(72) Inventor: Geoffrey Wayne Krissansen, Auckland (NZ)

(21) Appl. No.: 14/535,945

(22) Filed: Nov. 7, 2014

Related U.S. Application Data
(63) Continuation of application No. 13/703,115, filed on Apr. 22, 2013, now abandoned.

(30) Foreign Application Priority Data
Jun. 10, 2010 (NZ) 586074

(51) Int. Cl. C07K 7/08; A61K 47/48; C07K 7/06

(52) U.S. Cl. CPC C07K 7/08; C07K 7/06; A61K 47/48246; C07K 2319/00; A61K 48/00

(57) ABSTRACT

Novel peptides and constructs for carrying compounds across a cell membrane. Also provided are methods for carrying compounds across the cell membrane, antagonizing or destroying X-protein of HBV, treatment and/or management of HBV infection, treatment and/or prevention of HCC, and degradation of a target protein.

### CROSS-REFERENCE TO RELATED APPLICATIONS

The present application is a continuation of U.S. patent application Ser. No. 13/703,115 filed on Apr. 22, 2013, now abandoned; which is the national stage of International Application PCT/NZ2011/000102 filed on Jun. 10, 2011; which claims the priority of New Zealand Patent Application No. 586074 filed on Jun. 10, 2010. The contents of the prior applications are incorporated by reference herein in their entirety.

### SEQUENCE LISTING

A computer readable file containing a sequence listing is being electronically co-filed herewith via EFS-Web. The computer readable file, submitted under 37 CFR §1.821(e), will also serve as the copy required by 37 §CFR 1.821(c). The file (filename “28B8036.TXT”) was created on Nov. 3, 2014 and has a size of 47,835 bytes.

The content of the computer readable file is hereby incorporated by reference in its entirety.

### BACKGROUND

1. Field of the Invention

The present invention relates to novel peptides, constructs containing same and uses therefor.

2. Background Information

The plasma membrane of eukaryotic cells has poor permeability to many chemical compounds, significantly reducing their efficacy, for example as therapeutics or experimental reagents. Technologies have been developed to improve the cell permeability of chemical compounds, including the use of lipid-, polycationic-, nanoparticle- and peptide-based methods. However, these technologies are not without problem. For example, cell permeable carrier peptides, which are typically conjugated to a chemical compound for delivery to a target cell, may be large and expensive to manufacture, making them commercially non-viable. Large carrier peptides may also interfere with the conformation of the molecule which they carry, reducing efficacy of those compounds. In addition, carrier peptides typically do not distinguish between adherent and non-adherent cells. Thus, difficulties can arise in ensuring the conjugated chemical compound is adequately delivered to target cells, particularly when used in vivo. For example, in many cases, upon delivery to an animal, blood cells may mop up the conjugate before it is able to reach a target site. Further, it is often a requirement to deliver a chemical compound to the nucleus of a cell, and some carrier peptides do not localise to the nucleus.

The human hepatitis B virus (HBV) contains four ORFs encoding the viral envelope, core and E antigen, a polymerase protein and the X-protein. The X-protein of HBV is a complex pleiotropic molecule, generally divided into six domains denoted A-F, based on homology to other X-proteins in the hepadnaviridae family (Kumar, Jayasuryan, & Kumar, 1996; Misra, Mukherji, & Kumar, 2004).^{1,2} The various functions of the X-protein have not been fully elucidated, but it is believed to confer some survival advantage to the virus.

Hepatitis B is transmitted through infected blood or body fluids containing blood. Transmission can occur via blood transfusions, sexual contact, needle stick injuries and both vertical and horizontal transmission. The focus of recent efforts has largely been to prevent infection by vaccination, given the high rates of infection of the HBV virus. The vaccine consists of one of the viral envelope proteins such as HbsAg. After a full course of vaccination seroprotection can be as high as 95-100% in healthy children and young adults (Zanetti, Van Damme, & Shouval, 2008).³ Unfortunately, vaccination cannot help those who are already chronically infected and will not reduce the burden of the disease for many years to come. In addition many high risk countries lack the funds for a wide-scale vaccination programme, and there is a lack of disease awareness within the population leading to poor reception to vaccination.

The rising incidence of hepatocellular carcinoma (HCC; liver cancer) due to chronic infection of over 400 million people world-wide with HBV is a major global health problem.⁴ Evidence indicates that the HBV X-gene (encoding the X-protein) initiates and/or accelerates the development of hepatocellular carcinoma (HCC).^{5,6} Despite extensive exploration for novel anti-cancer drugs and therapeutic strategies, there has been little success in improving the treatment of HCC. Only surgery offers a cure, but tumor resection is feasible for less than 15% of patients, and recurrence rates remain as high as 50% after tumor resection due to the aggressive features of HCC including rapid growth, resistance to chemotherapy, and lack of effective adjunct therapy after surgery.^{7,8}

The X-protein is reported to be a cofactor in the development of HCC.⁶ High sustained expression of the X-gene either led to tumour development in 84% of male transgenic mice,⁹ or rendered them more susceptible to the tumorigenic effects of hepatocarcinogens.^{10,11} The X-protein transforms NIH3T3 fibroblasts and murine hepatocytes.¹² It is a tumour promoter, which promotes the proliferation of hepatocytes, and predisposes an individual to the detrimental effects of hepatocarcinogens.¹⁰ It is detectable in the sera and livers of patients with hepatitis, and HCC.^{13,14} Expression of the X-protein in hepatocytes led to the accumulation of cells in the S phase through the inhibition of DNA repair and checkpoints.^{15,16} The X-protein upregulates factors that favour tumorigenesis, and tumour survival such as survivin,¹⁷ and TGF-β1,¹⁸ and stimulates angiogenesis by upregulating vascular endothelial growth factor (VEGF).¹⁹ The above may partly explain its ability to also accelerate hepatitis C virus-induced liver pathogenesis,²⁰ and the development of HCC.

Agents that disrupt the expression and/or function of the X-protein inhibit the proliferation and survival of the hepatitis B virus, and inhibit X-protein induced tumorigenesis, and therefore may prevent or delay the development of HCC in patients who are chronically infected with HBV. Thus, RNAi against the X-gene reduced the proliferation of X-gene expressing HCC cells, and reduced anchorage-independent growth in soft agar, and tumour development in nude mice.²¹ It inhibited hepatitis B virus replication.²²⁻²⁴ However, stabilized peptides or peptidomimetics and small molecule inhibitors derived from them that specifically inhibit the function of the X-protein or cause its degradation may be preferable as they can be delivered orally, and could have reduced risk of off target toxicity.

Bibliographic details of the publications referred to herein are collected at the end of the description.

Object

It is an object of the present invention to provide novel peptides, conjugates comprising same, and/or uses therefor, or at least to provide the public with a useful choice.

### SUMMARY

In one embodiment of the invention, there is provided a peptide comprising the amino acid sequence LCLRP (SEQ ID NO 1), or a functionally equivalent variant thereof.

In one embodiment of the invention, there is provided a peptide comprising the amino acid sequence LCLRPVG (SEQ ID NO 2), or a functionally equivalent variant thereof.

In one embodiment of the invention, there is provided a peptide comprising the amino acid sequence MAARLCCQLDPARDVLCLRP (SEQ ID NO 3), or a functionally equivalent variant thereof.

In one embodiment, the peptide or functionally equivalent variant further comprises at its N-terminus, one or more amino acids which correspond consecutively to amino acids 1 to 15 of a native X-protein, and/or at its C-terminus, one or more amino acids which correspond consecutively to amino acids 21 to 35 of a native X-protein.

In one embodiment, the peptide consists of the amino acid sequence LCLRPVG (SEQ ID NO 2).

In another embodiment, the peptide consists of the amino acid sequence LCLRPVGAE (SEQ ID NO 4).

In another embodiment, the peptide consists of the amino acid sequence LCLRPVGAESR (SEQ ID NO 5).

In another embodiment, the peptide consists of the amino acid sequence LCLRPVGAESRGRPV (SEQ ID NO 90).

In another embodiment, the peptide consists of the amino acid sequence LCLRPVGAESRGRPVSGPFG (SEQ ID NO 6).

In another embodiment, the peptide consists of the amino acid sequence MAARLCCQLDPARDVLCLRP (SEQ ID NO 3).

In a second broad aspect the invention provides a peptide comprising the amino acid sequence MAARLCCQ (SEQ ID NO 7), or a functionally equivalent variant thereof.

In one embodiment, the peptide or functionally equivalent variant further comprises at its C-terminus, one or more amino acids which correspond consecutively to amino acids 9 to 15 of a native X-protein.

In one embodiment, the peptide or functionally equivalent variant further comprises at its C-terminus, one or more amino acids which correspond consecutively to amino acids 9 to 35 of a native X-protein.

In one embodiment, the invention provides a peptide comprising the amino acid sequence MAARLCCQLDPARDV (SEQ ID NO 8), or a functionally equivalent variant thereof.

In one embodiment, the peptide consists of the amino acid sequence MAARLCCQ (SEQ ID NO 7). In another embodiment, the peptide consists of the amino acid sequence MAARLCCQLDPARDV (SEQ ID NO 8).

In a third broad aspect, the invention provides nucleic acids encoding a peptide or functionally equivalent variant of the first or second broad aspects.

In a fourth broad aspect, the invention provides a nucleic acid vector comprising a nucleic acid of the third broad aspect.

In a fifth broad aspect the invention provides the use of a peptide or functionally equivalent variant of the first or second broad aspect as a cell membrane permeable carrier for a compound.

In a sixth broad aspect, the invention provides a construct comprising at least a peptide or functionally equivalent variant of the first or second broad aspect and at least one compound desired to be delivered to a cell.

In one embodiment, the peptide consists of the amino acid sequence LCLRP (SEQ ID NO 1). In another embodiment the peptide consists of the amino acid sequence LCLRPVG (SEQ ID NO 2). In another embodiment, the peptide consists of the amino acid sequence LCLRPVGAE (SEQ ID NO 4), LCLRPVGAESR (SEQ ID NO 5), LCLRPVGAESRGRPV (SEQ ID NO 90), LCLRPVGAESRGRPVSGPFG (SEQ ID NO 6), or MAARLCCQLDPARDVLCLRP (SEQ ID NO 3).

In another embodiment, the peptide consists of the amino acid sequence MAARLCCQ (SEQ ID NO 7). In another embodiment, the peptide consists of the amino acid sequence MAARLCCQLDPARDV (SEQ ID NO 8).

In one embodiment, the compound is a nucleic acid, a peptide nucleic acid, a polypeptide, a carbohydrate, a peptidomimetic, a small molecule inhibitor, proteoglycan, lipid, a lipoprotein, glycolipid, a natural product, or glycomimetic.

In one embodiment, the compound is a peptide comprising the amino acid sequence LVRSPAPCKFFTSA (SEQ ID NO 9) or a functionally equivalent variant thereof. In other embodiments, the compound is a peptide comprising the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), or GGCRHKLVRSPAPCKFFTSA (SEQ ID NO 91) or a functionally equivalent variant thereof. In another embodiment, the compound is the oxygen-dependent degradation (ODD) of HIF 1α (MLAPYIPM) (SEQ ID NO 13) or functionally equivalent variant thereof.

In a seventh broad aspect, the invention provides a nucleic acid encoding a construct of the sixth broad aspect.

In an eighth broad aspect, the invention provides a vector comprising a nucleic acid of the seventh broad aspect.

In a ninth broad aspect the invention provides a method for increasing the cell membrane permeability of a compound, the method comprising connecting a peptide or functionally equivalent variant of the first or second broad aspect of the invention to the compound.

In a tenth broad aspect the invention provides a method of delivering a compound to a cell, the method comprising contacting a construct comprising the compound and a peptide or functionally equivalent variant of the first or second broad aspect of the invention with the cell or a composition comprising the cell.

In a related broad aspect, the invention provides a method of delivering a compound to a cell, the method comprising administering to a subject a construct comprising the compound and a peptide or functionally equivalent variant of the first or second broad aspect of the invention.

In one embodiment, the compound is delivered to the cytoplasm of a cell. In another embodiment, the compound is delivered to the nucleus of a cell.

In a thirteenth broad aspect, the invention provides the use of a peptide comprising the amino acid sequence LCLRPVGAESRGRPV (SEQ ID NO 90), or a functionally equivalent variant thereof, as an antagonist of X-protein function.

In one embodiment, the invention provides the use of a peptide comprising the amino acid sequence LCLRPVGAESRGRPVSGPFG (SEQ ID NO 6), or a functionally equivalent variant thereof, as an antagonist of X-protein function.

In a fourteenth broad aspect, the invention provides a method for antagonising or disrupting X-protein function, the method comprising contacting a peptide comprising the amino acid sequence LCLRPVGAESRGRPV (SEQ ID NO 90) or a functionally equivalent variant thereof with an X-protein or a composition comprising an X-protein.

In one embodiment, the invention provides a method for antagonising or disrupting X-protein function, the method comprising contacting a peptide comprising the amino acid sequence LCLRPVGAESRGRPVSGPFG (SEQ ID NO 6) or a functionally equivalent variant thereof with an X-protein or a composition comprising an X-protein.

In a related broad aspect, the invention provides a method for antagonising or disrupting X-protein function, the method comprising administering to a subject a peptide comprising the amino acid sequence LCLRPVGAESRGRPV (SEQ ID NO 90) or a functionally equivalent variant thereof.

In one embodiment, the invention provides a method for antagonising or disrupting X-protein function, the method comprising administering to a subject a peptide comprising the amino acid sequence LCLRPVGAESRGRPVSGPFG (SEQ ID NO 6) or a functionally equivalent variant thereof.

In a fifteenth broad aspect, the invention provides a method for the treatment of hepatitis B virus infection, the method comprising administering to a subject a peptide comprising the amino acid sequence LCLRPVGAESRGRPV (SEQ ID NO 90) or a functionally equivalent variant thereof.

In one embodiment, the invention provides a method for the treatment of hepatitis B virus infection, the method comprising administering to a subject a peptide comprising the amino acid sequence LCLRPVGAESRGRPVSGPFG (SEQ ID NO 6) or a functionally equivalent variant thereof.

In a sixteenth broad aspect, the invention provides a method for treating or inhibiting the development of hepatocellular carcinoma, the method comprising administering to a subject a peptide comprising the amino acid sequence LCLRPVGAESRGRPV (SEQ ID NO 90) or a functionally equivalent variant thereof.

In one embodiment, the invention provides a method for treating or inhibiting the development of hepatocellular carcinoma, the method comprising administering to a subject a peptide comprising the amino acid sequence LCLRPVGAESRGRPVSGPFG (SEQ ID NQ 6) or a functionally equivalent variant thereof.

In a seventeenth broad aspect, the invention provides a construct comprising at least a degradation molecule and a peptide comprising the amino acid sequence LCLRPVGAESRGRPV (SEQ ID NO 90) or a functionally equivalent variant thereof.

In one embodiment, the invention provides a construct comprising at least a degradation molecule and a peptide comprising the amino acid sequence LCLRPVGAESRGRPVSGPFG (SEQ ID NO 6) or a functionally equivalent variant thereof.

In one embodiment, the degradation molecule is a peptide comprising the amino acid sequence LVRSPAPCKFFTSA (SEQ ID NO 9) or a functionally equivalent variant thereof. In other embodiments, the degradation molecule is a peptide comprising the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), or GGCRHKLVRSPAPCKFFTSA (SEQ ID NO 91) or a functionally equivalent variant thereof. In another embodiment, the degradation molecule is a peptide comprising the amino acid sequence MLAPYIPM (SEQ ID NO 13) or a functionally equivalent variant thereof.

In an eighteenth broad aspect, the invention provides a method for treating HBV infection, the method comprising administering to a subject a construct of the seventeenth broad aspect of the invention.

In a nineteenth broad aspect, the invention provides a method for treating or inhibiting the development of hepatocellular carcinoma, the method comprising administering to a subject a construct of the seventeenth broad aspect of the invention.

In a twentieth broad aspect, the invention provides a method for antagonising or disrupting X-protein function, the method comprising contacting a construct of the seventeenth broad aspect of the invention with an X-protein or a composition comprising an X-protein.

In a twenty first broad aspect, the invention provides a method for targeting delivery of a compound to adherent cells in a mixed population of adherent and non-adherent cells, the method comprising contacting a construct of the sixth broad aspect with a mixed population of cells or a composition comprising a mixed population of cells.

In a related broad aspect, the invention provides a method for targeting delivery of a compound to adherent cells in a subject, the method comprising administering a construct of the sixth broad aspect to the subject.

In a twenty seventh broad aspect, the invention provides a construct comprising at least a targeting molecule and a peptide comprising the amino acid sequence LVRSPAPCKFFTSA (SEQ ID NO 9) or a functionally equivalent variant thereof. In other embodiments, the construct comprises a peptide comprising the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), or GGCRHKLVRSPAPCKFFTSA (SEQ ID NO 91) or a functionally equivalent variant thereof.

In one embodiment, the targeting molecule targets the X-protein. In one particular embodiment, the targeting molecule comprises the amino acid sequence LCLRPVGAESRGRPV (SEQ ID NO 90) or a functionally equivalent variant thereof. In another embodiment, the targeting molecule comprises the amino acid sequence LCLRPVGAESRGRPVSGPFG (SEQ ID NO 6) or a functionally equivalent variant thereof.

In another embodiment, the targeting molecule targets B-raf. In one particular embodiment, the targeting molecule comprises the amino acid sequence LNVTAPTPQQLQAFKNEVGVLRK (SEQ ID NO 89) or a functionally equivalent variant thereof. In other embodiments, the targeting molecule comprises the amino acid sequence TTHNFVRKTFFTLAFCDFCRKLL (SEQ ID No 92) or SLPGSLTNVKALQKSPGPQRERK (SEQ ID No 93) or functionally equivalent variants of either.

In one particular embodiment, the construct targets B-raf and comprises the amino acid sequence RRRRRRRRHKLVRSPAPCKFFTSAGGLNVTAPTPQQLQAFKNEVGVLRK (SEQ ID NO 19) or a functionally equivalent variant thereof.

In another embodiment, the targeting molecule targets the protein N-Ras. In one embodiment, the targeting molecule comprises a peptide from Raf-1. In one embodiment, the targeting molecule comprises the amino acid sequence RKTFLKLA (SEQ ID No 94) or CCAVFRL (SEQ ID No 95) or a functionally equivalent variant of either.

In another embodiment, the targeting molecule targets the PDGF receptor. In one embodiment, the targeting molecule comprises a peptide from bovine papillomavirus E5 protein. In one embodiment, the targeting molecule comprises the amino acid sequence MPNLWFLLFLGLVAAMQLLLLLFLLLFFLVYWDHFECSCTGLPF (SEQ ID No. 96) or a functionally equivalent variant thereof.

In one particular embodiment, the construct comprises at least two targeting molecules.

In a twenty eighth broad aspect, the invention provides a nucleic acid encoding a construct of the twenty seventh broad aspect. The invention also provides a vector comprising such nucleic acid.

In a twenty ninth broad aspect, the invention provides the use of a peptide comprising the amino acid sequence LVRSPAPCKFFTSA (SEQ ID NO 9) or a functionally equivalent variant thereof for targeting a protein for degradation. In other embodiments, the peptide comprises the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), or GGCRHKLVRSPAPCKFFTSA (SEQ ID NO 91) or a functionally equivalent variant thereof.

In a thirtieth broad aspect, the invention provides a method of degrading a protein the method comprising providing a construct of the twenty seventh broad aspect, or a nucleic acid of the twenty eighth broad aspect to a composition comprising the protein.

In one embodiment, the composition comprising the protein includes a cell. In a related embodiment, the method comprises administering the construct or nucleic acid to a subject in need thereof.

In another broad aspect, the invention provides the use of a construct of the twenty seventh broad aspect or a nucleic acid of the twenty eighth broad aspect in the manufacture of a medicament for the treatment of disease associated with an undesirable level or activity of one or more proteins.

In another broad aspect, the invention provides a method for the treatment of melanoma the method comprising administering to a subject a construct comprising a targeting molecule and a peptide comprising the amino acid sequence LVRSPAPCKFFTSA (SEQ ID NO 9), or a functionally equivalent variant thereof, wherein the targeting molecule targets one or more of B-raf, N-Ras, and the PDGF receptor.

In a related aspect, the invention provides a method for the treatment of melanoma the method comprising administering to a subject a nucleic acid encoding a construct comprising a targeting molecule and a peptide comprising the amino acid sequence LVRSPAPCKFFTSA (SEQ ID NO 9), or a functionally equivalent variant thereof, wherein the targeting molecule targets one or more of B-raf, N-Ras, and the PDGF receptor.

In another aspect the invention provides the use of a construct comprising a targeting molecule and a peptide comprising the amino acid sequence LVRSPAPCKFFTSA (SEQ ID NO 9) or a functionally equivalent variant thereof, wherein the targeting molecule targets one or more of B-raf, N-Ras and the PDGF receptor in the manufacture of medicament for the treatment of melanoma.

In a related aspect the invention provides the use of a nucleic acid encoding a construct comprising a targeting molecule and a peptide comprising the amino acid sequence LVRSPAPCKFFTSA (SEQ ID NO 9) or a functionally equivalent variant thereof, wherein the targeting molecule targets one or more of B-raf, N-ras, and the PDGF receptor in the manufacture of medicament for the treatment of melanoma.

In one embodiment, the targeting molecule comprises the amino acid sequence LNVTAPTPQQLQAFKNEVGVLRK (SEQ ID NO 89) or a functionally equivalent variant thereof. In other embodiments, the targeting molecule comprises the amino acid sequence TTHNFVRKTFFTLAFCDFCRKLL (SEQ ID No 92) or SLPGSLTNVKALQKSPGPQRERK (SEQ ID No 93) or functionally equivalent variants of either.

In one particular embodiment, the construct comprises the amino acid sequence RRRRRRRRHKLVRSPAPCKFFTSAGGLNVTAPTPQQLQAFKNEVGVLRK (SEQ ID NO 19) or a functionally equivalent variant thereof.

In one embodiment, the targeting molecule comprises the amino acid sequence RKTFLKLA (SEQ ID No 94) or CCAVFRL (SEQ ID No 95) or a functionally equivalent variant of either.

In one embodiment, the targeting molecule comprises the amino acid sequence MPNLWFLLFLGLVAAMQLLLLLFLLLFFLVYWDHFECSCTGLPF (SEQ ID No. 96) or a functionally equivalent variant thereof.

In another aspect, the invention provides a nucleic acid encoding a construct of the seventeenth aspect. In another aspect, the invention provides a vector comprising said nucleic acid.

In another aspect, the invention provides a host cell comprising a nucleic acid or vector of the invention.

In another aspect, the invention provides the use of a nucleic acid encoding a peptide or construct of the invention, or a nucleic acid vector comprising said nucleic acid, for any purpose mentioned herein before. In addition, the invention provides methods as mentioned hereinbefore which utilise a nucleic acid encoding a peptide or construct of the invention, or a nucleic acid vector comprising said nucleic acid.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE DRAWINGS

These and other aspects of the present invention, which should be considered in all its novel aspects, will become apparent from the following description, which is given by way of example only, with reference to the accompanying figures.

A number of the figures illustrate cells or nuclei which have been labelled or stained in different colours. When reproduced in black and white, all the spots visible in these Figures represent cells or nuclei, in accordance with the label or stain used, unless otherwise stated. In addition, in these figures, the “merge” results illustrate cell staining coinciding with incidence of nucleus staining. Generally, the central areas represent the nucleus and the halo represents the cell as a whole (including the cytoplasm).

FIG. 1: Western blot analysis of the nuclear extract of HepG2 cells transfected to express the X-protein. HepG2 cells were left untransfected (lane 1), or transfected with pCDNA3.1-HBX (lane 2), pCDNA3-HBX-Myc (lane 3), and pCDNA3-GFP (lane 4). The membrane was probed with a mouse anti-human X-protein primary antibody (1:200 dilution), followed by a secondary goat anti-mouse HRP-conjugated antibody (1:10,000 dilution). The positions of the full-length 21 kDa, and truncated 17 kDa X-protein bands are indicated in the right-hand margin.

FIG. 2: Peptide 16-35 binds to the X-protein N-terminal peptide 1-50. The fluorescence emitted by short FITC-conjugated peptides encompassing aa 1-50 that had been bound to peptide 1-50-coated plates was graphed. Wells containing peptide 16-35 at 40 μg/ml gave a higher fluorescence, which is evidence that peptide 16-35 binds peptide 1-50.

FIG. 3: Peptides shorter than peptide 16-35 fail to bind peptide 1-50. Graph of ligand-binding, indicated by level of fluorescence, using short peptides based on peptide 16-35. The binding obtained with the shortened peptides was not comparable to the binding seen with parental peptide 16-35.

FIG. 4: Peptide 16-35 fused to the ODD of HIF-1a binds peptide 1-50. The graph shows that binding of the two X-protein-ODD fusion peptides where peptide 16-35 is located at either the front or end of the targeting peptide. Parental peptide 16-35, and the non-binding peptide 21-40 were included as positive and negative controls, respectively.

FIG. 5: Western blot analysis of the nuclear compartment of HepG2 cells transfected to express the X-protein and treated with X-protein-ODD fusion peptides. HepG2 cells were left untransfected (lane 1), or transfected with pCDNA3.1-HBX (lanes 2, 5, and 8), pCDNA3-HBX Myc (lanes 3, 6, and 9), and pCDNA3-GFP (lanes 4, 7, and 10). Transfectants were left untreated (lane 1) or were treated with the X-protein-ODD fusion peptides with the ODD tag at the front (lanes 5-7), or at the end (lanes 8-10). The positions of the full-length 21 kDa, and truncated 17 kDa X-protein bands are indicated in the left-hand margin. The X-protein-ODD fusion peptides have degraded the X-proteins.

FIG. 6: Western blot analysis of the nuclear fraction of HepG2 cells transfected to express the X-protein and treated with X-protein oligomerization-instability domain fusion peptides. HepG2 cells were left untransfected (lane 1), or transfected with pCDNA3.1-HBX (lanes 2, 5, and 8), pCDNA3-HBX Myc (lanes 3, 6, and 9), and pCDNA3-GFP (lanes 4, 7, and 10). Transfectants were left untreated (lane 1) or were treated with the X-protein oligomerization-instability fusion peptides with the instability domain at the front (lanes 5-7), or at the end (lanes 8-10). The positions of the full length 21 kDa, and truncated 17 kDa X-protein bands are indicated in the left-hand margin. The X-protein oligomerization-instability fusion peptides have degraded the X-proteins.

FIG. 7: The oligomerization domain peptide (aa 16-35) inhibits truncated X-protein-mediated apoptosis of HepG2 cells. HepG2 cells were engineered to express the truncated X-protein by transfection with the pCDNA3.1-HBX plasmid. The X-protein oligomerization peptide (aa 16-35) was added to the cells at either 3 h, 3 and 24 h, or 3, 24, and 48 h post transfection, and the cells were cultured for a total of 51 h. Control transfectants were not treated with peptide. Cells were stained with annexin-V to record apoptosis, propidium iodide to record necrosis, and DAPI to visualize cell nuclei. The annexin-V and DAPI results were merged to illustrate the total number of cells, and the number of cells undergoing apoptosis, ie the proportion of cells undergoing apoptosis. The oligomerization domain peptide (aa 16-35) inhibited truncated X-protein-mediated apoptosis of HepG2 cells.

FIG. 8: The oligomerization domain peptide (aa 16-35) inhibits full-length X-protein-mediated apoptosis of HepG2 cells. HepG2 cells were engineered to express the full-length X-protein by transfection with the pCDNA3.1-HBX Myc plasmid. The X-protein oligomerization peptide (aa 16-35) was added to the cells at either 3 h, 3 and 24 h, or 3, 24, and 48 h post transfection, and the cells were cultured for a total of 51 h. Control transfectants were not treated with peptide. Cells were stained with annexin-V to record apoptosis, propidium iodide to record necrosis, and DAPI to visualize nuclei. The annexin-V and DAPI results were merged to illustrate the total number of cells, and the number of cells undergoing apoptosis, ie the proportion of cells undergoing apoptosis. The oligomerisation domain peptide (aa 16-35) inhibited full-length X-protein-mediated apoptosis of HepG2 cells.

FIG. 9: The control peptide (aa 140-153) does not inhibit truncated X-protein-mediated apoptosis of HepG2 cells. HepG2 cells were engineered to express the truncated X-protein by transfection with the pCDNA3.1-HBX plasmid. The X-protein control peptide (aa 140-153) was added to the cells at either 3 h, 3 and 24 h, or 3, 24, and 48 h post transfection, and the cells were cultured for a total of 51 h. Control cells were not treated with peptide. Cells were stained with annexin-V to record apoptosis, propidium iodide to record necrosis, and DAPI to visualize nuclei. The annexin-V and DAPI results were merged to illustrate the total number of cells, and the number of cells undergoing apoptosis ie the proportion of cells undergoing apoptosis. The control peptide (aa 140-153) did not inhibit truncated X-protein-mediated apoptosis of HepG2 cells.

FIG. 10: The control peptide (aa 140-153) does not inhibit full-length X-protein-mediated apoptosis of HepG2 cells. HepG2 cells were engineered to express the truncated X-protein by transfection with the pCDNA3.1-HBX Myc plasmid. The X-protein control peptide (aa 140-153) was added to the cells at either 3 h, 3 and 24 h, or 3, 24, and 48 h post transfection, and the cells were cultured for a total of 51 h. Control cells were not treated with peptide. Cells were stained with annexin-V to record apoptosis, propidium iodide to record necrosis, and DAPI to visualize nuclei. The annexin-V and DAPI results were merged to illustrate the total number of cells, and the number of cells undergoing apoptosis ie the proportion of cells undergoing apoptosis. The control peptide (aa 140-153) did not inhibit full-length X-protein-mediated apoptosis of HepG2 cells.

FIG. 11: Disruption of the tertiary structure of the X-protein by the oligomerization domain peptide (aa 16-35). HepG2 cells were transfected with either the pCDNA3.1-HBX (lanes 1 and 4), pCDNA3-HBX Myc (lanes 2 and 5), or pCDNA3-GFP (lanes 3 and 6) plasmids, and were either left untreated (lanes 1-3), or treated thrice over 48 h with the oligomerization domain peptide (aa 16-35) (lanes 4-6). The nuclear subfraction was resolved by SDS-PAGE under non-reducing conditions, and Western blotted with a mouse anti-human X-protein primary antibody. Molecular weight markers are shown in the left-hand margin.

FIG. 12: Peptides aa 1-20 and 16-35 and are cell-permeable. Four FITC-labelled peptides encompassing aa 1-20, 16-35, 21-40 and 34-53 from the N-terminal region of the X-protein were incubated with HepG2 cells and their uptake by the cells recorded using a Nikon E600 fluorescence microscope. Cell nuclei were stained with DAPI.

FIG. 13: Confocal microscopy confirms that peptides aa 1-20 and 16-35 and are cell-permeable. Four FITC-labelled peptides encompassing aa 1-20, 16-35, 21-40 and 34-53 from the X-protein were incubated with HepG2 cells and their uptake by the cells recorded using a Leica TCS-SP2 confocal microscope. Cell nuclei were stained with DAPI.

FIG. 14: Confocal slicing of cells reveals that peptide aa 16-35 is taken up into the cytoplasm and nucleus. FITC-labelled peptide aa 16-35 was incubated with HepG2 cells and its uptake by the cells recorded using a Leica TCS-SP2 confocal microscope. Multiple optical slices of the cell were taken from the bottom to the top of the cells. Cell nuclei were stained with DAPI.

FIG. 15: Short peptides aa 16-26, 16-24 and 16-22 derived from peptide aa 16-35 are also cell-permeable. FITC-labelled peptides aa 16-26, 16-24 and 16-22 were incubated with HepG2 cells and their uptake by the cells recorded using a Nikon E600 fluorescence microscope. Cell nuclei were stained with DAPI.

FIG. 16: Confocal microscopy confirms that peptides aa 16-26, 16-24 and 16-22 are cell-permeable. FITC-labelled peptides aa 16-26, 16-24 and 16-22 were incubated with HepG2 cells and their uptake by the cells recorded using a Leica TCS-SP2 confocal microscope. Cell nuclei were stained with DAPI.

FIG. 17: Confocal slicing of cells reveals that peptide aa 16-22 is taken up into the cytoplasm and nucleus. FITC-labelled peptide aa 16-22 was incubated with HepG2 cells and its uptake by the cells recorded using a Leica TCS-SP2 confocal microscope. Multiple optical slices of the cell were taken from the bottom to the top of the cells. Cell nuclei were stained with DAPI.

FIG. 18: Entry of X-protein peptides aa 16-22, 16-35 into HepG2 cells is dependent on heparin binding. HepG2 cells were pretreated with 5 μg/ml cytochalasin D and 2 μg/ml heparin, and then incubated with the FITC-labelled cell-permeable peptides aa 16-22, and aa 16-35 for 3 h. Peptide uptake by the cells was recorded using a Nikon E600 fluorescence microscope. Cell nuclei were stained with DAPI. The results were merged to illustrate the numbers of cells that took up the FITC-labelled peptides compared with the total number of cells, ie the proportion of cells that took up the peptides.

FIG. 19: Short peptides aa 1-15 and 16-20 are also cell-permeable. FITC-labelled peptides aa 1-15, 16-20, and 16-22 were incubated with HepG2 cells and their uptake by the cells recorded using a Nikon E600 fluorescence microscope. Cell nuclei were stained with DAPI. The results were merged to illustrate the numbers of cells that took up the FITC-labelled peptides compared with the total number of cells ie the proportion of cells that took up the peptides. Short peptides aa 1-15 and 16-20 are cell-permeable.

FIG. 20: Confocal microscopy confirms that peptides aa 1-15, 16-20 and 16-22 are cell-permeable. FITC-labelled peptides aa 1-15, 16-20, and 16-22 were incubated with HepG2 cells and their uptake by the cells recorded using a Leica TCS-SP2 confocal microscope (upper panel). Confocal slicing of cells reveals that peptides aa 1-15 and 16-22 are taken up into the cytoplasm and nucleus. Multiple optical slices of the cells were taken from the bottom to the top of the cells. Cell nuclei were stained with DAPI.

FIG. 21: Short peptide aa 1-8 is cell-permeable. FITC-labelled peptides aa 1-8, and aa 9-15 were incubated with HepG2 cells and their uptake by the cells recorded using a Nikon E600 fluorescence microscope. Cell nuclei were stained with DAPI. The results were merged to illustrate the numbers of cells that took up the FITC-labelled peptides compared with the total number of cells ie the proportion of cells that took up the peptide. Short peptide aa 1-8 is cell-permeable.

FIG. 22: The X-protein cell-permeable peptide 16-22 is able to enter the HepG2, C32 and DU145 cell lines, but not the nonadherent cell line TK1. The adherent HepG2, C32, and DU145 cell lines, and nonadherent cell line TK1 were incubated with the fluoresceinated (FITC-labelled) X-protein cell-permeable peptide 16-22 for 3 h and visualized by fluorescence microscopy. Cell nuclei were stained with DAPI. The results were merged to illustrate the numbers of cells that took up the FITC-labelled peptide compared with the total number of cells ie the proportion of cells that took up the peptide. The X-protein cell-permeable peptide 16-22 was able to enter the HepG2, C32 and DU145 cell lines, but not the nonadherent cell line TK1.

FIG. 23: The X-protein cell-permeable peptide 16-22 is taken up less efficiently into the H441, COS-7, COS-1 and Rinm5f cell lines. The adherent H441, COS-7, COS-1, and Rinm5f cell lines were incubated with the FITC-labelled X-protein cell-permeable peptide 16-22 for 3 h and visualized by fluorescence microscopy. Cell nuclei were stained with DAPI. The results were merged to illustrate the numbers of cells that took up the FITC-labelled peptides compared with the total number of cells ie the proportion of cells that took up the peptide. The X-protein cell-permeable peptide 16-22 was taken up less efficiently into the H441, COS-7, COS-1 and Rinm5f cell lines.

FIG. 24: The X-protein cell-permeable peptide 16-22 is unable to enter nonadherent peripheral blood mononuclear cells. Peripheral blood mononuclear cells were incubated for 3 h in 8-chamber slides with the FITC-labelled cell-permeable peptide 16-22, and viewed by fluorescence microscopy (lower panels). Cell nuclei were stained with DAPI (upper panels). Three different fields are presented.

FIG. 25: The X-protein cell-permeable peptide 16-22 is able to enter adherent monocytes, and potentially adherent platelets. Peripheral blood mononuclear cells were incubated for 3 h in 8-chamber slides with the FITC-labelled cell-permeable peptide 16-22, and visualized by fluorescence microscopy (middle panels). Cell nuclei were stained with DAPI (upper panels). The slides were washed with media to remove nonadherent cells. Two different fields are presented. Images were merged (lower panels) to illustrate the distribution of FITC-labelled peptides with respect to the nuclei. Cells that had taken up the peptide are indicated by the arrows. Small fluorescent specks may represent adherent platelets that have taken up the peptide.

FIG. 26: The X-protein cell-permeable peptide 16-22 does not enter nonadherent erythrocytes and platelets. Blood was collected from a finger prick, suspended in 10 mM citrate buffer, incubated with the FITC-labelled 16-22 peptide, and a blood smear prepared on a glass slide. The cells were viewed by light microscopy (upper panels), and by fluorecence microscopy (lower panels).

FIG. 27: Adherent TK1 T cells take up the X-protein cell-permeable peptide 16-22. TK1 cells were adhered to MAdCAM-1-coated glass slides (left-hand panels), or to the slides directly (right-hand panels), and incubated with the FITC-labelled X-protein cell-permeable peptide 16-22. Cell nuclei were stained with DAPI. Two fields are presented. The results were merged to illustrate the numbers of cells that took up the FITC-labelled peptide compared with the total number of cells ie the proportion of cells that took up the peptide. Adherent TK1 T cells took up the X-protein cell-permeable peptide 16-22.

FIG. 28: The X-protein cell-permeable peptide 16-22 can carry a foreign peptide into cells. TK-1 cells activated with 2 mM Mn++ in HBSS buffer were left to attach to MAdCAM-1-coated 8 well glass chamber slides. Biotinylated R9YDRREY (SEQ ID NO 14) and LCLRPVGGYDRREY (SEQ ID NO 15) peptides were added at 50 μM, and 30 min later the cells were washed, and analyzed. Cells were stained with streptavidin-FITC, and the nuclei were stained with DAPI. The YDRREY (SEQ ID NO 16) peptide is foreign, being derived from the cytoplasmic domain of the β7 integrin.

FIG. 29: Peptide aa 16-22 carries rabbit IgG into HepG2 cells. The X-protein cell-permeable peptide LCLRPVG (SEQ ID NO 2) fused to a polyglutamine stretch (biotin-LCLRPVGGGRRRQQQQQQRRR) (SEQ ID NO 17) was conjugated to FITC-labelled rabbit IgG using transglutaminase. The peptide-rabbit IgG conjugate, the peptide, and FITC-labelled rabbit IgG were added to HepG2 cells, and cell uptake of the FITC-labelled rabbit IgG cargo was assessed by fluorescence microscopy. Cell nuclei were stained with DAPI. The results were merged to illustrate the numbers of cells that took up the FITC-labelled rabbit IgG compared with the total number of cells ie the proportion of cells that took up the antibody. Peptide aa 16-22 carried the rabbit IgG into HepG2 cells.

FIG. 30A: Peptide aa 16-22 carries an 18mer oligonucleotide into HepG2 cells. The X-protein cell-permeable peptide LCLRPVG (SEQ ID NO 2) fused to a polyglutamine stretch (biotin-LCLRPVGGGRRRQQQQQQRRR) (SEQ ID NO 17) was conjugated to the Tex615-labelled 18mer oligonucleotide 5AmMC12-GAGCTGCACGCTGCCGTC (SEQ ID NO 18) containing a 5′-amino group using transglutaminase. The peptide-18mer oligo conjugate was added to HepG2 cells, and its uptake was assessed by fluorescence microscopy after 30 min, 3, and 24 h. Uptake of unconjugated oligo was assessed after 3 h. Cell nuclei were stained with DAPI. The results were merged to illustrate the numbers of cells that took up the Tex615-labelled oligonucleotide compared with the total number of cells, i.e., the proportion of cells that took up the oligonucleotide. Peptide aa 16-22 carried the 18mer oligonucleotide into HepG2 cells.

FIG. 30B: The 18 mer oligo described in the legend to FIG. 30A was conjugated at 2.3, 4.5, and 8.7 μM oligo to 10 μM carrier peptide, giving final concentrations of oligo in solution of 0.14, 0.3 and 0.6 μM. Uptake was assessed as described above.

FIG. 31: A B-Raf targeting peptide kills melanoma cells. The B-Raf X-protein fusion peptide (biotin-RRRRRRRRHKLVRSPAPCKFFTSAGGLNVTAPTPQQLQAFKNEVGVLRK.) (SEQ ID NO 19) containing a polyArg8 carrier peptide fused to a B-Raf dimerization domain, and the X-protein instability domain was added to the C32 and WM-266-4 melanoma cells to final concentrations of 10 μM and 20 μM, respectively, followed by incubation for 3 h at 37° C.

Cells were incubated with the control cell-permeable peptide biotin-RRRRRRRRMAARLCCQLDPARDVLCLRP (SEQ ID NO 20) which does not cause apoptosis. Cell apoptosis was detected by staining cells with Annexin-V fluos. Nuclei were stained with DAPI. The Annexin-V and DAPI results were merged to illustrate the proportion of cells killed by the B-Raf targeting peptide. The B-Raf targeting peptide killed the melanoma cells.

FIG. 32: Peptide 16-30 binds to the X-protein N-terminal peptide 1-50. The fluorescence emitted by short FITC-conjugated X-protein peptides aa 16-30, 20-35, and 16-35 that had been bound to peptide 1-50-coated plates was graphed. Wells containing peptide 16-30 at 10, 20, and 40 μg/ml gave a higher fluorescence, which is evidence that peptide 16-30 binds peptide 1-50. A and B, repeat experiments.

FIG. 33: A polyclonal anti-B-raf antibody carried into WM-266-4 melanoma cells by the X-protein aa 16-22 carrier peptide causes the cells to undergo apoptosis. The X-protein cell-permeable peptide LCLRPVG (SEQ ID NO 2) fused to a polyglutamine stretch (biotin-LCLRPVGGGRRRQQQQQQRRR) (SEQ ID NO 17) was conjugated to a polyclonal rabbit anti-human B-raf antibody (HPA001328; Sigma) using transglutaminase. The carrier peptide-rabbit anti-B-raf antibody conjugate (1 μg/ml), the unconjugated carrier peptide and unconjugated anti-B-raf antibody (1 μg/ml) were added to WM-266-4 melanoma cells, and cell apoptosis was assessed after 3 h by addition of Annexin-V fluos, with visualization by fluorescence microscopy. Untreated cells were included as an additional control. Cell nuclei were stained with DAPI. The results were merged to illustrate the numbers of cells that underwent apoptosis compared with the total number of cells ie the proportion of cells that underwent apoptosis. The polyclonal anti-B-raf antibody carried into melanoma cells with the X-protein carrier peptide caused the cells to undergo apoptosis.

### DETAILED DESCRIPTION

The following is a description of the present invention, including preferred embodiments thereof, given in general terms. The invention is further elucidated from the disclosure given under the heading “Examples” herein below, which provides experimental data supporting the invention, specific examples of various aspects of the invention, and means of performing the invention.

The inventors have surprisingly identified peptide motifs derived from the X-protein of the hepatitis B Virus (HBV) which are cell permeable. These peptides may be used as cell-permeable carriers to deliver chemical compounds to cells (including the cytoplasm and nucleus of cells). The peptides have a number of advantages including: they are small in size, making them relatively economical to manufacture; they target adherent cells, which may increase efficacy of delivery of compounds to such cells as they are not taken up by non-adherent cells, such as blood cells. The inventors contemplate the use of the peptides for delivery of therapeutic compounds to subjects, as well as for research purposes.

The inventors have also surprisingly identified peptide motifs that are required for dimerization of the X-protein. These peptides antagonize X-protein function and the inventors contemplate their use in the treatment and/or management of HBV infection, the treatment and/or prevention of HCC, as well as for research purposes. In addition, they may be conjugated to a degradation molecule (for example the instability domain of the X-protein, or the oxygen-dependent degradation (ODD) domain of Hypoxia-Inducible Factor 1α (HIF-1α) to target and destroy the X-protein. Such constructs may also have advantage in treatment and/or management of HBV infection, the treatment and/or prevention of HCC, as well as being useful for research purposes.

Further, the inventors have demonstrated that the instability domain of the X-protein can be used to elicit degradation of a protein. Thus, the instability domain can be linked to a molecule which targets a specific protein to form a construct which targets or marks the protein for degradation. Such constructs may have use in a number of applications, for research purposes and therapeutically. For example, it could be used to inhibit or reduce undesirable protein activity or protein levels in a cell.

As used herein, the term “treatment” is to be considered in its broadest context. The term does not necessarily imply that a subject is treated until total recovery. Accordingly, “treatment” broadly includes, for example, the prevention, amelioration or management of one or more symptoms of a disorder, the severity of one or more symptoms and preventing or otherwise reducing the risk of developing secondary complications. For example, in the case of HBV infection, treatment may include reduction in viral load or ongoing management of viral load, and preventing or otherwise reducing the risk of developing secondary complications resulting from HBV infection, in particular the development of HCC. “Prevention” of disease should not be taken to imply that disease development is completely prevented, and include delay of disease development.

### Peptides and Functionally Equivalent Variants

In one embodiment, the invention provides peptides comprising the amino acid sequence LCLRP (SEQ ID NO 1) or functionally equivalent variants of said peptides. This core amino acid sequence maps to amino acid position 16-20 of the mature X-protein of HBV (GenBank accession number Y18857; isolate HBV-C6).

Peptides of this embodiment of the invention may further comprise at the N-terminus, one or more amino acids which correspond to amino acids 1 to 15 of a native X-protein, and/or at the C-terminus one or more amino acids corresponding to amino acids 21 to 35 of a native X-protein, such that the peptide sequence corresponds to a region of consecutive amino acids from the native protein. They may also include heterologous amino acids at the N- or C-terminus.

In another embodiment, the invention provides peptides comprising the amino acid sequence MAARLCCQ (SEQ ID NO 7) or functionally equivalent variants of said peptides. This core amino acid sequence maps to the N-terminal amino acids 1-8 of the mature X-protein of HBV (GenBank accession number Y18857).

Peptides of this embodiment of the invention may further comprise at the C-terminus one or more amino acids corresponding to amino acids 9 to 35 of a native X-protein, such that the peptide sequence corresponds to a region of consecutive amino acids from the native protein. They may also include heterologous amino acids at the N- or C-terminus. In one embodiment the peptide comprises the amino acid sequence MAARLCCQLDPARDV (SEQ ID NO 8).

Skilled persons will readily appreciate amino acids at positions 1 to 35 of a native X-protein, having regard to the information herein and other published sequence information. By way of example, see OenBank accession number Y18857 also provides exemplary sequence information. In addition Gunther S, Fischer L, Pult I, Sterneck M, Will H. Naturally occurring variants of hepatitis B virus. Adv Virus Res. 1999; 52:25-137 provides sequence information for a number of X-proteins. Further, examples of useful sequence information is provided in Table 1, below.

TABLE 1

| Protein | | | |
|---|---|---|---|
| Accession No. | Locus | Sequence | SEQ ID NO |
| Q81163 | HBVC8 | MAARVCCQLDPARDVLCLRPVGAESRGRPVSGPFG | 21 |
| P0C689 | HBVC5 | As above | 21 |
| P12936 | HBVC3 | As above | 21 |
| P0C686 | HBVC1 | As above | 21 |
| Q9YZR6 | HBVC2 | MAARMCCQLDPARDVLCLRPVGAESRGRPVSGPFG | 22 |
| Q93195 | HBVD7 | MAARLCCQLDPARDVLCLRPVGAESRGRPFSGPFG | 23 |
| Q67863 | HBVC4 | MAARVCCQLDPARDVLCLRPVGAESRGRPVSRPFG | 24 |
| Q67877 | HBVD6 | MAARLCCQLDPARDVLCLRPVGAESRGRPFSGPLG | 25 |
| P24026 | HBVD2 | MAARLCCQLDPARDVLCLRPVGAESRGRPFSGPLG | 26 |
| P0C687 | HBVC9 | MAARLCCQEDPTRDVLCLRPVGAESRGRINSGPLG | 27 |
| P0C681 | HBVD5 | MAARLCCQLDPARDVLCLRPVGAESRGRPFSGPLG | 28 |
| Q913A9 | HBVC7 | MAARLCCQLDPARDVLCLRPVGAESRGRPFSGPLG | 29 |
| O91531 | HBVA7 | MAARLCCQLDPSRDVLCLRPVGAESRGRPLSGPLG | 30 |
| Q9E6S8 | HBVC0 | MAARLCCQLDPARDVLCLRPVGAESRGRPVSGSLG | 31 |
| Q9PX75 | HBVB7 | MAARLCCQLDPARDVLCLRPVGAESRGRPLPGPLG | 32 |
| P20975 | HBVB2 | MAARLCCQLDPARDVLCLRPVGAESRGRPLPGPLG | 33 |
| P0C685 | HBVB3 | MAARLCCQLDPARDVLCLRPVGAESRGRPLPGPLG | 34 |
| P20976 | HBVB1 | MAARLCCQLDPARDVLCLRPVGAESRGRPLPGPLG | 35 |
| P17102 | HBVA4 | MATRLCCQLDPSRDVLCLRPVGAESRGRPLSGPLG | 36 |
| Q9PXA2 | HBVB5 | MAARLCCQLDPARDVLCLRPVGAESRGRPLPGPLG | 37 |
| P03165 | HBVD3 | MAARLCCQLDPARDVLCLRPVGAESRGRPFSGSLG | 38 |
| P20977 | HBVB4 | MAARLCCQLDPARDVLCLRPVGAESRGRPFPGPLG | 39 |
| Q99HR6 | HBVF4 | MAARMCCQLDPARDVLCLRPVGAESRGRPLPGPLG | 40 |
| Q67923 | HBVB6 | MAARVCCQLDPARDVLCLRPVGAESRGRPLPGPLG | 41 |
| P69714 | HBVA2 | MAARLYCQLDPSRDVLCLRPVGAESRGRPLSGPLG | 42 |
| P69713 | HBVA3 | MAARLYCQLDPSRDVLCLRPVGAESRGRPLSGPLG | 43 |
| Q9IBI5 | HBVG3 | MAARLCCQLDPSRDVLCLRPVSAESSGRPLPGPFG | 44 |
| P0C678 | HBVB8 | MAARLCCQLDTARDVLCLRPVGAESRGRPLPGPLG | 45 |
| Q05499 | HBVF1 | MAARMCCKLDPARDVLCLRPIGAESRGRPLPGPLG | 46 |
| Q80IU5 | HBVE4 | MAARLCCQLDPARDVLCLRPVGAESCGRPVSGSLG | 47 |
| Q8JMY3 | HBVF2 | MAARLCCQLDPARDVLCLRPVGAESRGRSLSGSLG | 48 |
| Q9J5S3 | HBVOR | MAARLCCQLDTARDVLCLRPVGAESRGRPFSGSVG | 49 |
| Q9QAX0 | HBVE3 | MAARLCCQLDPARDVLCLRPVSAESCGRPVSGSLG | 50 |
| Q69604 | HBVE1 | MAARLCCQLDPARDVLCLRPVSAESCGRPVSGSLG | 51 |
| Q4R1S9 | HBVA8 | MAARLYCQLDSSRDVLCLRPVGAESRGRPFSGPLG | 52 |
| Q91C38 | HBVA6 | MAARLYCQLDSSRDVLCLRPVGAESRGRPLAGPLG | 53 |
| Q8JMY5 | HBVH1 | MAARLCCQLDPARDVLCLRPVGAESCGRPLSWSLG | 54 |
| Q80IU8 | HBVE2 | MAARLCCQLDPARDVLCLRPVSAESCGRSVSGSLG | 55 |
| P12912 | HBVCP | MAARLCCQLDTSRDVLCLRPVGAESCGRPFSGPL | 56 |
| Q69607 | HBVF6 | MAARLCCQLDPARDVLCLRPVGAESSGRTLPGSLG | 57 |
| Q8JMZ5 | HBVH3 | MAARLCCQLDPARDVLCLRPVGAESCGRPLS | 58 |
| Q8JN06 | HBVH2 | MAARLCCQLDPARDVLCLRPVGAESCGRPLS | 59 |
| P87743 | HBVGB | MAARMCCQLDPSQDVLCLRPVGAESRGRP | 60 |
| Q9YJT2 | HBVG0 | MAARLCCQLDPARDVLCLRPVGAEPCRRPVSG | 61 |
| Q4R1S1 | HBVA9 | MAARLYCQLDSSRNVLCLRPVGAESCGRPLSGPVG | 62 |

By way of example, in one embodiment, a peptide of the invention consists of the amino acid sequence LCLRP (SEQ ID NO 1). In another embodiment, the peptide consists of the amino acid sequence LCLRPVG (SEQ ID NO 2). In another embodiment, the peptide consists of the amino acid sequence LCLRPVGAE (SEQ ID NO 4). In another embodiment, the peptide consists of the amino acid sequence LCLRPVGAESR (SEQ ID NO 5). In another embodiment, the peptide consists of the amino acid sequence LCLRPVGAESRGRPV (SEQ ID NO 90). In another embodiment, the peptide consists of the amino acid sequence LCLRPVGAESRGRPVSGPFG (SEQ ID NO 6). In yet another embodiment, the peptide consists of the amino acid sequence MAARLCCQ

(SEQ ID NO 7). In another embodiment, the peptide consists of the amino acid sequence MAARLCCQLDPARDV (SEQ ID NO 8). In yet another embodiment, the peptide consists of the amino acid sequence MAARLCCQLDPARDVLCLRP (SEQ ID NO 3).

In other embodiments, a peptide of the invention consists of the amino acid sequence LCLRPVGAESRGRPVSGPF (SEQ ID NO 71), LCLRPVGAESRGRPVSGP (SEQ ID NO 70), LCLRPVGAESRGRPVSG (SEQ ID NO 69), LCLRPVGAESRGRPVS (SEQ ID NO 68), LCLRPVGAESRGRPV (SEQ ID NO 67), LCLRPVGAESRGRP (SEQ ID NO 66), LCLRPVGAESRG (SEQ ID NO 65), LCLRPVGAER (SEQ ID NO 64), LCLRPVGAE (SEQ ID NO 4), or LCLRPVGA (SEQ ID NO 63).

As noted herein before, the invention includes functionally equivalent variants of the peptides of the invention. The phrase “functionally equivalent variants” as used herein, includes those peptides in which one or more conservative amino acid substitutions have been made, while substantially retaining the desired function of the peptide. By way of example, in the case of peptides to be used to deliver a compound to a cell, the peptide and a functionally equivalent variant thereof will have the ability to move across a cell membrane, preferably carry the compound across a cell membrane, to enter a cell. By way of further example, in the case of peptides to be used as antagonists of the X-protein, the peptide and a functionally equivalent variant thereof will have the ability to antagonise or disrupt X-protein function. By way of further example, in the case of peptides used to target a protein for degradation, the peptide and a functionally equivalent variant thereof will have the ability to label a protein such that it is a target for subsequent degradation.

A peptide(s) of the invention and its functionally equivalent variant(s) may be referred to herein collectively as “peptide(s)”. Accordingly, where not specifically mentioned, references to a “peptide” or “peptides” of the invention herein should be taken to include reference to functionally equivalent variants thereof.

As used herein the phrases “move across a cell membrane”, “carry a compound across a cell membrane”, “cell membrane translocation” and like phrases, should be taken broadly to encompass transport of the peptide, a compound to be delivered to a cell, and/or a conjugate comprising such peptide and compound from the outside of a cell to the inside of the cell. It should not be taken to imply a particular mode or mechanism of transport across or through the cell membrane. Similarly the phrase “increasing the cell membrane permeability of a compound” should be taken broadly to mean that there has been at least some increase or improvement in the ability of the compound to move across a cell membrane.

It should be appreciated that a “functionally equivalent variant” may have a level of activity higher or lower than the peptide of which it is a variant. In various embodiments of the invention a functionally equivalent variant has at least 50%, at least 60%, at least 70%, at least 80%, at least 90%, at least 95% or at least 99% of the level of activity of the peptide of which it is a variant.

Skilled persons will readily appreciate the desired function and be able to assess function and determine the level of activity of a peptide or functionally equivalent variant thereof peptide of the invention, based on the information contained herein, and using techniques known in the art. However, by way of example, in the case of use of a peptide to deliver a compound to a cell, the peptide or variant will have the ability to move across a cell membrane, preferably carry the compound across a cell membrane, to enter a cell and this function and the level of activity may be assessed based on uptake of the variant (preferably, constructs comprising the variant) into the cell, for example, using the techniques described in the “Examples” section herein after. In other embodiments of the invention (described elsewhere herein) it is desired that a peptide of the invention act as an antagonist of the X-protein. In such cases, the peptide or a functionally equivalent variant thereof will have the ability to antagonise or disrupt X-protein function, and its function and its level of activity may be assessed, for example, using the techniques described in the “Examples” section herein after. In other embodiments (described elsewhere herein) it is desired that a peptide of the invention marks a protein for degradation and preferably elicits protein degradation. In such cases, the peptide or a functionally equivalent variant thereof will have the ability to label a protein for subsequent degradation and its function and its level of activity may be assessed using standard techniques having regard to the nature of the target protein. In one embodiment, the techniques described in the “Examples” section herein after may be used.

As used herein “conservative amino acid substitution(s)” should be taken broadly to mean substitution of amino acids that have similar biochemical properties. Persons skilled in the art will appreciate appropriate conservative amino acid substitutions based on the relative similarity between different amino acids, including the similarity of the amino-acid side chain substituents (for example, their size, charge, hydrophilicity, hydrophobicity and the like). By way of example, a conservative substitution includes substitution of one aliphatic amino acid for another aliphatic amino acid, substitution of an amino acid with a hydroxyl- or sulphur-containing side chain with another amino acid with a hydroxyl- or sulphur-containing side chain, substitution of an aromatic amino acid with another aromatic amino acid, substitution of a basic amino acid with another basic amino acid, or substitution of an acidic amino acid with another acid amino acid. By way of further example, “conservative amino acid substitution(s)” include:
substitution of Glycine, Alanine, Valine, Leucine, or Isoleucine, one for another
substitution of Serine, Cysteine, Theronine, or Methionine, one for another
substitution of Phenylalanine, Tyrosine, or Tryptophan, one or another
substitution of Histidine, Lysine, or Arginine, one for another
substitution of Aspartic acid, Glutamic acid, Asparagine or Glutamine, one for another

Functionally equivalent variants containing amino acid substitutions in accordance with this aspect of the invention will preferably retain at least 70%, 80%, 90%, 95% or 99% amino acid sequence similarity to the original peptide. In one embodiment, the functionally equivalent variant has at least 70%, 80% 90%, 95% or 99% sequence identity with the original peptide.

Peptides of the invention (including functionally equivalent variants) may be composed of L-amino acids, D-amino acids or a mixture thereof and may include non-naturally occurring amino acids.

It should be understood that peptides of the invention (including functionally equivalent variants), are “isolated” or “purified” peptides. An “isolated” or “purified” peptide is one which has been identified and separated from the environment in which it naturally resides, or artificially synthesized. It should be appreciated that these terms do not reflect the extent to which the peptide has been purified or separated from an environment in which it naturally resides.

A peptide of the invention may be isolated from natural sources, or preferably derived by chemical synthesis (for example, fmoc solid phase peptide synthesis as described in Fields G B, Lauer-Fields J L, Liu R Q and Barany G (2002) Principles and Practice of Solid-Phase peptide Synthesis; Grant G (2002) Evaluation of the Synthetic Product. Synthetic Peptides, A User's Guide, Grant G A, Second Edition, 93-219; 220-291, Oxford University Press, New York) or genetic expression techniques, methods for which are readily known in the art to which the invention relates. Standard recombinant DNA and molecular cloning techniques are described for example in: Sambrook, and Maniatis, Molecular Cloning: A Laboratory Manual, Second Edition, Cold Spring Harbor Laboratory Press, Cold Spring Harbor, N.Y. (1989); Silhavy et al., Experiments with Gene Fusions, Cold Spring Harbor Laboratory Press, Cold Spring Harbor, N.Y. (1984); and, Ausubel et al., Current Protocols in Molecular Biology, published by Greene Publishing Assoc. and Wiley-Interscience (1987). The inventor's also contemplate production of a peptide of the invention by an appropriate transgenic animal, microbe, or plant.

### Nucleic Acids

To the extent that a peptide of the present invention may be produced by recombinant techniques the invention provides nucleic acids encoding peptides of the invention and vectors comprising nucleic acids encoding peptides of the invention, which may aid in cloning and expression of peptides. Certain nucleic acids and vectors may also be of use to a therapeutic end as herein after detailed.

It should be understood that a nucleic acid in accordance with the invention, is an “isolated” or “purified” nucleic acid. An “isolated” or “purified” nucleic is one which has been identified and separated from the environment in which it naturally resides, or artificially synthesized. It should be appreciated that these terms do not reflect the extent to which the nucleic has been purified or separated from the environment in which it naturally resides. Nucleic acids of use in accordance with the invention may be isolated from natural sources, or preferably derived by chemical synthesis or recombinant techniques which will be readily known to persons skilled in the art.

Those of general skill in the art to which the invention relates will readily be able to identify a variety of nucleic acids which encode the peptides and functionally equivalent variants of the invention on the basis of the amino acid sequences provided herein, the genetic code and the understood degeneracy therein and published X-protein nucleic acid sequences (for example, see Guo, Y. and Hou, J. Establishment of the consensus sequence of hepatitis B virus prevailing in the mainland of China. Zhonghua Min Guo Wei Sheng Wu Ji Mian Yi Xue Za Zhi 19: 189-2000, 1999). However, by way of example, the following nucleic acids are suitable:

(SEQ ID NO 72)
ctt tgt cta cgt ccc (peptide comprising region
16-20 of X-protein)
(SEQ ID NO 73)
ctt tgt cta cgt ccc gtc ggc (peptide comprising
region 16-22 of X-protein)
(SEQ ID NO 74)
ctt tgt cta cgt ccc gtc ggc gct gaa (peptide
comprising region 16-24 of X-protein)
(SEQ ID NO 75)
ctt tgt cta cgt ccc gtc ggc gct gaa tcc cgc
(peptide comprising region 16-26 of X-protein)
(SEQ ID NO 76)
ctt tgt cta cgt ccc gtc ggc gct gaa tcc cgc gga
cga ccc gtc tcg ggg ccg ttt ggg
(peptide comprising region 16-35 of X-protein)
(SEQ ID NO 77)
atg gct gct agg ctg tgc tgc caa ctg gat cct gcg
cgg gac gtc ctt tgt cta cgt ccc
(peptide comprising region 1-20 of X-protein)

Nucleic acid vectors will generally contain heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and are either capable or not capable of replicating in such cell. Recombinant constructs comprising nucleic acids of the invention may be used, for example, in the cloning, sequencing, and expression of nucleic acid sequences of the invention. Additionally, recombinant constructs or vectors of the invention may be used to a therapeutic end.

Those of skill in the art to which the invention relates will recognise many constructs suitable for use in the present invention. However, the inventors contemplate the use of cloning vectors such as pUC and pBluescript and expression vectors such as pCDM8, adeno-associated virus (AAV) or lentiviruses to be particularly useful.

The constructs may contain regulatory sequences such as promoters, operators, repressors, enhancers, termination sequences, origins of replication, and other appropriate regulatory sequences as are known in the art. Further, they may contain secretory sequences to enable an expressed protein to be secreted from its host cell. In addition, expression constructs may contain fusion sequences (such as those that encode a heterologous amino acid sequence) which lead to the expression of inserted nucleic acid sequences of the invention as fusion proteins or peptides. Heterologous amino acid sequences of use may include, for example, those which can aid in subsequent isolation and purification of the peptide (for example, ubiquitin, his-tag, a c-myc tag, a GST tag, or biotin), or those which assist the activity of the peptide (for example, an additional sequence which aids in transport across a cell membrane, such as a poly arginine sequence, tat, or penetratin). Heterologous amino acid sequences may also include peptide linkers which aid in linking the peptide to another compound to form a construct of the invention

In accordance with the invention, transformation of a nucleic acid vector into a host cell can be accomplished by any method by which a nucleic acid sequence can be inserted into a cell. For example, transformation techniques include transfection, electroporation, microinjection, lipofection, adsorption, and biolistic bombardment.

As will be appreciated, transformed nucleic acid sequences of the invention may remain extrachromosomal or can integrate into one or more sites within a chromosome of a host cell in such a manner that their ability to be expressed is retained.

Any number of host cells known in the art may be utilised in cloning and expressing nucleic acid sequences of the invention. For example, these include but are not limited to microorganisms such as bacteria transformed with recombinant bacteriophage DNA, plasmid DNA or cosmid DNA expression vectors; yeast transformed with recombinant yeast expression vectors; insect cell systems infected with recombinant virus expression vectors (e.g., baculovirus); animal cell systems such as CHO (Chinese hamster ovary) cells using the pEE14 plasmid system; plant cell systems infected with recombinant virus expression vectors (e.g., cauliflower mosaic virus, CaMV; tobacco mosaic virus, TMV) or transformed with recombinant plasmid expression vectors (e.g., Ti plasmid). Those host cells detailed herein after under “Examples” are found to be particularly useful.

A recombinant peptide in accordance with the invention may be recovered from a transformed host cell, or culture media, following expression thereof using a variety of techniques standard in the art. For example, detergent extraction, sonication, lysis, osmotic shock treatment and inclusion body purification. The protein may be further purified using techniques such as affinity chromatography, ion exchange chromatography, filtration, electrophoresis, hydrophobic interaction chromatography, gel filtration chromatography, and chromatofocusing.

Additional or alternative methodology for recombinant expression of peptides of the invention may be obtained from Sambrook, and Maniatis, Molecular Cloning: A Laboratory Manual, Second Edition, Cold Spring Harbor Laboratory Press, Cold Spring Harbor, N.Y. (1989), for example.

### Carriers

In one embodiment of the invention, the peptides of the invention may be used as carriers to transport compounds across a cell membrane into a cell. Thus, the invention provides methods for delivering a compound to a cell as well as methods for increasing the cell membrane permeability of a compound to be delivered to a cell by connecting it to a peptide of the invention. It also provides constructs comprising a carrier peptide and at least one compound desired to be delivered to a cell.

The at least one compound may be any compound desired to be delivered to a cell. Such compounds include those which may provide a therapeutic or diagnostic benefit, or compounds of use for research purposes. In certain embodiments, the compounds are nucleic acids, peptide nucleic acids, polypeptides (including for example, fusion proteins), carbohydrates, peptidomimetics, small molecule inhibitors, chemotherapeutic drugs, anti-inflammatory drugs, antibodies, single chain Fv fragments (SCFV), lipids, proteoglycans, glycolipids, lipoprotein, glycomimetics, natural products, or fusion proteins. Where the compound is a nucleic acid it may be DNA, RNA, cDNA, double-stranded, single-stranded, sense, antisense, or circular, including DNAzymes, iRNA, siRNA, miRNA, piRNA, lcRNA, and ribozymes, phagemid, aptamer for example. Skilled persons may readily appreciate further examples of compounds in accordance with this embodiment of the invention.

In one particular embodiment, the compound is a peptide which is adapted to degrade the X-protein or target the X-protein for degradation. By way of example, the compound is a peptide comprising the amino acid sequence LVRSPAPCKFFTSA (SEQ ID NO 9) (or a functionally equivalent variant thereof). In other embodiments, the compound is a peptide comprising the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), or GGCRHKLVRSPAPCKFFTSA (SEQ ID NO 91) or a functionally equivalent variant thereof. “Functionally equivalent variant(s)” of these sequences (when referred to here or anywhere else herein) include, for example, peptides derived from any of the known HBV variants including those described in Table 2 herein. Exemplary sequences include:

TABLE 2

| Protein | | | |
|---|---|---|---|
| Accession | | | SEQ |
| No. | Locus | Sequence | ID No |
| P0C689 | HBVC5 | GGCRH----------------NFFTSA | 99 |
| P12936 | HBVC3 | GGCRHKLVCSPAPCNFFTSA | 100 |
| P0C686 | HBVC1 | GGCRHKLVCSPAPCNFFTSA | 101 |
| Q9YZR6 | HBVC2 | GGCRHKLVCSPAPCNFFTSA | 102 |
| O93195 | HBVD7 | GGCRHKLVCAPAPCNFFTSA | 103 |
| Q67863 | HBVC4 | GGCRHKLVCSPAPCNFFHLC | 104 |
| Q67877 | HBVD6 | GGCRHKLVCAPAPCNFFTSA | 105 |
| P24026 | HBVD2 | GGCRHKLVCAPAPCNFFTSA | 106 |
| P0C687 | HBVC9 | GGCRHKLVCSPAPCNFFTSA | 107 |
| P0C681 | HBVD5 | GGCRHKLVCAPAPCNFFTSA | 108 |
| Q913A9 | HBVC7 | GGCRHKLVCSPAPCNFFTSA | 109 |
| O91531 | HBVA7 | GGCRHKLVCAPAPCNFFTSA | 110 |
| Q9E6S8 | HBVC0 | GGCRHKLVCVPAPCNFFTSA | 111 |
| Q9PX75 | HBVB7 | GGCRHKLVCSPAPCNFFTSA | 112 |
| P20975 | HBVB2 | GGCRHKLVCSPAPCNFFTSA | 113 |
| P0C685 | HBVB3 | GGCRHKLVCSPAPCNFFTSA | 114 |
| P20976 | HBVB1 | GGCRHKLVCSPAPCNFFTSA | 115 |
| P17102 | HBVA4 | GGCRHKLVCAPAPCNFFTSA | 116 |
| Q9PXA2 | HBVB5 | GGCRHKLVCPPAPCNFFTSA | 117 |
| P03165 | HBVD3 | GGCRHKLVCAPAPCNFFTSA | 118 |
| P20977 | HBVB4 | GGCRHKLVCSPAPCNFFTSA | 119 |
| Q99HR6 | HBVF4 | GGCRHKLVCSPAPCNFFTSA | 120 |
| Q67923 | HBVB6 | GGCRHKLVCSPAPCNFFTSA | 121 |
| P69714 | HBVA2 | GGCRHKLVCAPAPCNFFTSA | 122 |
| P69713 | HBVA3 | GGCRHKLVCAPAPCNFFTSA | 123 |
| Q9IB15 | HBVG3 | GGCRHKLVCAPAPCNFFTSA | 124 |
| PQC678 | HBVB8 | GGCRHKLVCSPAPCNFFTSA | 125 |
| Q05499 | HBVF1 | GGCRHKLVCSPAPCNFFTSA | 126 |
| Q801U5 | HBVE4 | GGCRHKLVCVPAPCNFFTSA | 127 |
| Q8JMY3 | HBVF2 | GGCRHKLVCSPAPCNFFTSA | 128 |
| Q9J5S3 | HBVOR | GGCRHKLVCSPAPCNFFTSA | 129 |
| Q9QAX0 | HBVE3 | GGCRHKLVCAPAPCNFFTSA | 130 |
| Q69604 | HBVE1 | GGCRHKLVCAPAPCNFFTSA | 131 |
| Q4R1S9 | HBVA8 | GGCRHKLVCAPSSCNFFTSA | 132 |
| Q91C38 | HBVA6 | GGCRHKLVFAPSSCNFFTSA | 133 |
| Q8JMY5 | HBVH1 | GGCRHKLVCSPAPCNFFTSA | 134 |
| Q80IU8 | HBVE2 | GGCRHKLVCAPAPCNFFTSA | 135 |
| P12912 | HBVCP | GGCRHKLVCTPAPCNFFTSA | 136 |
| Q69607 | HBVF6 | GGCRHKLVCSPAPCNFFTSA | 137 |
| Q8JMZ5 | HBVH3 | GGCRHKLVCSPAPCNFFTSA | 138 |
| Q8JNO6 | HBVH2 | GGCRHKLVCSPAPCNFFTSA | 139 |
| P87743 | HBVGB | GGCRHKLVCSPAPCNFFTSA | 140 |
| Q9YJT2 | HBVGO | GGCRHKLVCAPAPCNFFTSA | 141 |
| Q4R1S1 | HBVA9 | GGCRHKLVCAPFSCNFFTSA | 142 |

In another embodiment, the compound is a peptide comprising the sequence of the oxygen-dependent degradation (ODD) of HIF 1α MLAPYIPM (SEQ ID NO 13) (or a functionally equivalent variant thereof).

Constructs of this embodiment of the invention are useful as antagonists of X-protein function and thus in the treatment of HBV infection, including complications arising from HBV infection, such as hepatocellular carcinoma, as will be described further herein after.

In another embodiment, the compound is a peptide comprising peptide forming at least a part of B-raf. In one particular embodiment the peptide forming part of B-raf is a peptide comprising the amino acid sequence LNVTAPTPQQLQAFKNEVGVLRK (SEQ ID NO 89) or a functionally equivalent variant thereof.

In one particular embodiment, the construct includes the peptide LCLRPVGAESRGRPV (SEQ ID NO 90) and the peptide RHKLVRSPAPCKFFTSA (SEQ ID NO 12). In another particular embodiment, the construct includes the peptide LCLRPVGAESRGRPVSGPFG (SEQ ID NO 6) and the peptide RHKLVRSPAPCKFFTSA (SEQ ID NO 12).

In another particular embodiment, the construct includes the peptide LCLRPVGAESRGRPV (SEQ ID NO 90) and the peptide MLAPYIPM (SEQ ID NO 13). In another particular embodiment, the construct includes the peptide LCLRPVGAESRGRPVSGPFG (SEQ ID NO 6) and the peptide MLAPYIPM (SEQ ID NO 13).

The carrier peptide and at least one compound to be delivered to a cell may be “connected” to each other by any means which allows the peptide to carry the compound across a cell membrane into a cell while retaining at least a level of the function and structure of the compound. The word “connected” or like terms should be taken broadly to encompass any form of attachment, bonding, fusion or association between the carrier peptide and the at least one compound (for example, but not limited to, covalent bonding, ionic bonding, hydrogen bonding, aromatic stacking interactions, amide bonds, disulfide bonding, chelation) and should not be taken to imply a particular strength of connection. The carrier peptide and the at least one compound may be connected in an irreversible or a reversible manner, such that upon entry into a cell the compound is released from the carrier peptide.

The at least one compound may be connected to the carrier peptide at its N-terminus, its C-terminus or at any other location. In one particular embodiment, the compound is connected to the carrier peptide at its N-terminus. In another particular embodiment, the compound is connected to the carrier peptide at its C-terminus.

Persons skilled in the art will readily appreciate methodology for manufacturing constructs of the invention, having regard to the nature of the at least one compound to be included in the construct. Such methods include manufacturing the peptide and compound separately and then connecting them, chemical synthesis of the construct, recombinant expression of the construct, and the like.

By way of example, in embodiments of the invention where the at least one compound is a peptide, the constructs may be produced in the form of fusion proteins using known recombinant expression or chemical synthesis techniques (as herein before described). The carrier peptide and the peptide (compound) to be delivered to a cell may also be manufactured separately and later connected to one another.

By way of further example, where the compound to be delivered to a cell is a nucleic acid, the carrier peptide and the nucleic acid may be made separately (using chemical synthesis or recombinant techniques, for example) and then connected via one of a number of known techniques.

By way of further example, in embodiments of the invention where the at least one compound is a carbohydrate, the carrier peptide and the carbohydrate may be made separately and then connected via one of a number of known techniques.

By way of further example, in embodiments of the invention where the at least one compound is a lipid, the carrier peptide and the lipid may be made separately and then connected via one of a number of known techniques.

By way of example only, the methodology described in WO 91/09958, WO 03/064459, WO 00/29427, WO 01/13957 may be used to manufacture various constructs of the invention.

It should be appreciated that while the carrier peptide and at least one compound to be delivered to a cell may be connected directly to one another, constructs of the invention may also utilise linker molecules which connect the at least one compound to the carrier peptide. Skilled persons will appreciate appropriate linker molecules of use in the invention. However, by way of example, the linker molecule may be a peptide. Examples of appropriate linker molecules are also provided in WO 91/09958, WO 03/064459, WO 00/29427, and WO 01/13957.

While it is not necessary for the performance of the invention, in one embodiment, the construct may further comprise at least one additional heterologous molecule. By way of example only, the heterologous molecule may be a molecule which may assist the activity of the construct (for example, the activity of the carrier peptide or the compound to be delivered to a cell, or both), protect the construct from degradation or otherwise increase the half-life of the construct, or aid in isolation and purification of the construct during manufacture. In one embodiment, the heterologous molecule is a molecule that may assist with cell membrane permeability (such as poly-arginine, Tat, or penetratin, for example). In another embodiment, the molecule may be a his-tag, a c-myc tag, a GST tag, or biotin, which may aid in isolation of a construct expressed recombinantly. In another embodiment, the heterologous molecule is a molecule that may assist in targeting the construct to a specific cell type. When used herein “targeting the construct to a specific cell type”, “specifically target a desired cell” and like phrases should not be taken to require 100% specificity, although this may be preferred. In another embodiment, the heterologous molecule is a molecule that may assist in targeting the construct to a specific molecular target. When used herein “targeting the construct to a specific molecular target”, “specifically target a desired molecule” and like phrases should not be taken to require 100% specificity, although this may be preferred.

It should be appreciated that a combination of different heterologous molecules may be used in a construct of the invention.

The heterologous molecules may be connected to the carrier peptide and/or compound to be delivered to a cell, or synthesised as a part of the construct, using any appropriate means (as described herein before in relation to manufacture of the constructs of the invention/connection of the peptide carrier to the at least one compound), having regard to the chemical nature of the heterologous molecule. In one embodiment, the heterologous molecules are peptide-based. However, those of skill in the art to which the invention relates will readily recognise molecules of an alternative nature which may be connected to or incorporated in the constructs of the invention. Examples of alternative molecules are provided, for example, in WO 91/09958, WO 03/064459, WO 00/29427, and WO 01/13957.

In certain embodiments, a construct of this aspect of the invention comprises the amino acid sequence:

(SEQ ID NO 78)
RRRRRRRRMLAPYIPMGGLCLRPVGAESRGRPVSGPFG;
(SEQ ID NO 79)
RRRRRRRRLCLRPVGAESRGRPVSGPFGGMLAPYIPM;
(SEQ ID NO 80)
RRRRRRRRHKLVRSPAPCKFFTSAGGLCLRPVGAESRGRPVSGPFG;
or,
(SEQ ID NO 81)
RRRRRRRRLCLRPVGAESRGRPVSGPFGGCRHKLVRSPAPCKFFTSA.

### Antagonists of X-Protein Function

In one embodiment, certain peptides of the invention (including functionally equivalent variants thereof), and constructs comprising the same, may be used as antagonists of X-protein function. Such peptides and constructs may thus be used to antagonise or disrupt X-protein function for research or therapeutic purposes, for example. In one particular embodiment, the inventors contemplate the use of the peptides or constructs comprising them in the treatment and management of HBV infection, and in the treatment (including prevention as herein before defined) of HCC.

As used herein “antagonist”, “antagonise”, “disrupt” X-protein function, and like terms should be taken broadly to refer to a reduction in X-protein function or activity. They should not be taken to imply complete inhibition of function or activity. Persons skilled in the art will readily appreciate methods which may be used to assess X-protein function and activity. However, by way of example, the methodology described in the “Examples” section herein after may be used.

In one embodiment, the peptides comprise the amino acid sequence LCLRPVGAESRGRPV (SEQ ID NO 90) or functionally equivalent variants thereof as herein described. In another embodiment, the peptides comprise the amino acid sequence LCLRPVGAESRGRPVSGPFG (SEQ ID NO 6) or functionally equivalent variants thereof as herein described.

In one embodiment, the invention provides a construct comprising a degradation molecule and a peptide comprising the amino acid sequence LCLRPVGAESRGRPV (SEQ ID NO 90) or a functionally equivalent variant thereof.

In one embodiment, the invention provides a construct comprising a degradation molecule and a peptide comprising the amino acid sequence LCLRPVGAESRGRPVSGPFG (SEQ ID NO 6) or a functionally equivalent variant thereof.

The degradation molecule may be any molecule which is adapted to degrade the X-protein or target the X-protein for degradation. In one embodiment, the degradation molecule is a peptide comprising the amino acid sequence LVRSPAPCKFFTSA (SEQ ID NO 9) or a functionally equivalent variant thereof. In other embodiments, the degradation molecule is a peptide comprising the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), or GGCRHKLVRSPAPCKFFTSA (SEQ ID NO 91) or a functionally equivalent variant thereof. In another embodiment, the degradation molecule is a peptide comprising the amino acid sequence MLAPYIPM (SEQ ID NO 13) or a functionally equivalent variant thereof. Skilled persons will readily appreciate other appropriate degradation molecules that may be used. However, by way of example any peptide that undergoes polyubiquination may be used.

The phrase “functionally equivalent variant” as used in this section should be taken to have the same general meaning as described herein before. In addition, a functionally equivalent variant of a specific peptide degradation molecule will retain at least some ability to degrade the X-protein or target it for degradation. The function and activity of a variant of a peptide degradation molecule may be using the methodology described in the “Examples” section herein, or any other appropriate methodology known in the art.

Peptides and constructs of this embodiment of the invention may be manufactured using the techniques described herein before for other peptides and constructs of the invention. The constructs may include any combination of one or more peptides of the invention and one or more degradation molecule. They may also include additional heterologous molecules and linkers, including for example, those previously described herein. They may also include additional motifs or molecules which may allow for the construct to be targeted to a specific cell type or to a specific molecular target and/or motifs or molecules that may assist with cell permeability, as described previously for other constructs of the invention. In one particular embodiment, the construct includes a poly arginine peptide (for example R₄ (SEQ ID NO: 143) to Rₙ, in one example R₈ (SEQ ID NO: 144)) and comprises the amino acid sequence: RRRRRRRR LCLRPVGAESRGRPVSGPFGGC RHKLVRSPAPCKFFTSA (R8-exemplary oligomerisation domain-instability domain) (SEQ ID NO 82); or RRRRRRRR LCLRPVGAESRGRPVSGPFG GMLAPYIPM (R8-exemplary oligomerisation domain-ODD of HIF) (SEQ ID NO 83).

### Targeting Proteins for Degradation

In one embodiment, peptides of the invention have the ability to target or mark a protein for subsequent degradation. These peptides include those comprising the sequence LVRSPAPCKFFTSA (SEQ ID NO 9) or functionally equivalent variants thereof. In certain embodiments, the peptide comprises the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), GGCRHKLVRSPAPCKFFTSA (SEQ ID NO 91), or a functionally equivalent variant thereof.

In accordance with this embodiment, the invention also includes constructs comprising at least one targeting molecule and a peptide comprising the amino acid sequence LVRSPAPCKFFTSA (SEQ ID NO 9) or a functionally equivalent variant thereof. In certain embodiments, the peptide comprises the amino acid sequence KLVRSPAPCKFFTSA (SEQ ID NO 10), HKLVRSPAPCKFFTSA (SEQ ID NO 11), RHKLVRSPAPCKFFTSA (SEQ ID NO 12), GGCRHKLVRSPAPCKFFTSA (SEQ ID NO 91), or a functionally equivalent variant thereof.

The phrase “functionally equivalent variant” as used in this section should be taken to have the same general meaning as described herein before. In addition, a functionally equivalent variant of the peptide referred to in the previous paragraph will retain at least some ability to degrade the X-protein (or another target protein) or target it for degradation. The function and activity of a variant of a peptide degradation molecule may be using the methodology described in the “Examples” section herein, or any other appropriate methodology known in the art.

Further, the phrase “target a protein for degradation” and like phrases are not intended to mean that all proteins present in a cell or composition are targeted or that all such targeted or labelled proteins will be subsequently degraded.

The targeting molecule may be any molecule which allows the construct to target a particular protein of interest for subsequent degradation. For example, it may be a protein implicated in disease. Preferably the targeting is specific. By way of example, the molecule may be a peptide, peptidomimetic, protein, antibody, single-chain Fv fragment (SCFV), aptamer, phagemid (phage display), or small molecule. Two or more targeting molecules may be incorporated into the construct, so that two or more target proteins may be marked for degradation.

In one embodiment, a targeting molecule which is a peptide can represent a dimerisation domain of its target or a site of interaction with a target protein.

In one embodiment, the targeting molecule is a peptide of the invention, preferably a peptide comprising LCLRPVGAESRGRPV (SEQ ID NO 90) or a functionally equivalent variant thereof. In another embodiment, the targeting molecule is a peptide comprising the sequence LCLRPVGAESRGRPVSGPFG (SEQ ID NO 6) or a functionally equivalent variant thereof. In these embodiments, the targeting molecule will target the X-protein for subsequent degradation.

In another embodiment, the targeting molecule is a peptide forming a part of the protein B-raf. In one specific embodiment, the peptide of B-raf comprises the oligomerisation domain of B-raf or a part thereof. In one embodiment, peptide of B-raf comprises or consists of the amino acid sequence LNVTAPTPQQLQAFKNEVGVLRK (SEQ ID No 89) or a functionally equivalent variant thereof. In another embodiment, the peptide of B-raf comprises the amino acid sequence TTHNFVRKTFFTLAFCDFCRKLL (SEQ ID No 92) and SLPGSLTNVKALQKSPGPQRERK (SEQ ID No 93), which represent amino acids 233 to 255 and 405 to 427 of B-raf, respectively, or a functionally equivalent variant thereof. In these embodiments, the targeting molecule will target B-raf for subsequent degradation. B-raf is implicated in melanoma and a construct of this embodiment of the invention may be used in the treatment thereof, for example.

In other embodiment, the targeting molecule targets the protein N-Ras or the PDGF receptor. For example, a peptide comprising the amino acid sequence RKTFLKLA (SEQ ID No 94) and CCAVFRL (SEQ ID No 95) representing amino acids 143 to 150 or 95 to 101 of the N-Ras binding sequences of Raf 1, respectively, or a functionally equivalent variant thereof. By way of further example, a peptide or peptide fragment of the 44 aa residue bovine papillomavirus E5 protein (MPNLWFLLFLGLVAAMQLLLLLFLLLFFLVYWDHFECSCTGLPF (SEQ ID No. 96) which interacts with the PDGF receptor, or a functionally equivalent variant thereof, may be used. In these embodiments, the targeting molecule will target N-ras or the PDGF receptor for subsequent degradation. N-ras and PDGF receptor are implicated in melanoma, and resistance to B-raf-based treatments and constructs of this embodiment of the invention may be used in the treatment thereof, for example.

In one embodiment, the construct may comprise one or more peptide targeting both N-Ras and B-raf. In another embodiment, the construct may comprise one or more peptide targeting both PDGF receptor and B-raf. In another embodiment, the construct may comprise one or more peptide targeting PDGF receptor, N-Ras, and B-raf.

The phrase “functionally equivalent variant” when used in relation to peptides of N-Ras, B-Raf, Raf 1, bovine papillomavirus E5 protein, and PDGF receptor, should be taken to have the same general meaning as described herein before, albeit having regard to the function of the peptide in this aspect of the invention; ie, the ability to target the protein for subsequent degradation.

Exemplary amino acid and nucleic acid sequences for N-Ras, B-raf and PDGF receptor may be found on GenBank; see for example NM_{—}002524 (N-Ras), NM_{—}004333 (B-raf) and NM_{—}002609 and NM_{—}006206 (PDGF receptor). Exemplary amino acid and nucleic acid sequences for Raf-1 and bovine papillomavirus E5 protein may be found on GenBank; see for example NM_{—}002880 (Raf-1) and NP_{—}056742 (E5 protein).

Skilled persons should readily appreciate a number of alternative targeting molecules and molecules that may be usefully targeted according to the invention. However, by way of example, and without limitation, the following molecules may be useful targets: in terms of cancer CD20, HER2 (ErbB-2), phospholipase C-gamma, c-Met, c-myc, insulin-like growth factor, ras, raf, mitogen-activated protein kinase (MEK), phosphatidylinositol 3-kinase (PI-3 kinase), 3-phosphoinositide-dependent protein kinase (PDK), mammalian target of rapamycin (mTOR), akt kinase, src, (histone deacetylase) HDAC, Bcl-2, XIAP, hsp90, Flt3, c-kit, cyclin-dependent kinase, lysophosphatidic acid (LPA) receptor, autotaxin, CD33, CD52, EGFR, VEGF, VEGF receptor, hypoxia-inducible factor, Delta-like 4, survivin, LMTK3, RANK ligand, indoleamine 2,3-dioxygenase (IDO), O-GlcNAc transferase, matrix metalloproteinase, Bcr-Abl, anaplastic lymphoma kinase, oestrogen receptor, aromatase, androgen receptor, poly ADP ribose polymerase, integrins, CTLA-4, and the molecules with which all the latter molecules interact may be used. Again, in terms of inflammatory disease, by way of example, and without limitation, molecules to be targeted may include leukocyte integrins (α4, β2, and β7 integrins) and their interactive partners, selectins and their partners, TNF and its receptor, prostaglandin D2 receptors DP1 and CRTH2, leukotriene receptors, chemokines and their receptors, proinflammatory cytokines and their receptors, cyclophilins, deoxyhypusine synthase and deoxyhypusine hydroxylase, cyclooxygenase, lipoxygenase, phospholipase A 2, phosphodiesterase, NF-κB, inflammasome, elastase, protease, matrix-metalloproteinase, T cell receptor, CD3 complex, toll-like receptor, G-protein-coupled receptors, inosine monophosphate dehydrogenase receptor, T cell costimulatory molecules, mitogen activated protein kinases, complement components and their receptors, interleukin-1β converting enzyme (ICE), TNF-α converting enzyme (TACE), MAP kinases (eg p38), and c-Jun N-terminal kinases (JNK).

Any of the above exemplary molecules that dimerize could be used to target themselves, otherwise interactive molecules and ligands, and derived peptides thereof, would serve as appropriate targeting molecules.

Such molecules and others may be described, for example in: Dancey J E, Chen H X. Strategies for optimizing combinations of molecularly targeted anticancer agents. Nat Rev Drug Discov. 2006 August; 5(8):649-59; Peng X, Pentassuglia L, Sawyer D B. Emerging anticancer therapeutic targets and the cardiovascular system: is there cause for concern? Circ Res. 2010 Apr. 2; 106(6):1022-34; Benson J D, Chen Y N, Cornell-Kennon S A, Dorsch M, Kim S, Leszczyniecka M, Sellers W R, Lengauer C. Validating cancer drug targets. Nature 2006 May 25; 441(7092):451-6.; Klein S, Levitzki A. Targeted cancer therapy: promise and reality. Adv Cancer Res. 2007; 97:295-319; Wu et al. Journal of Cancer Molecules 2:57-66, 2006; Simmons D L. What makes a good anti-inflammatory drug target? Drug Discov Today. 2006 March; 11(5-6):210-9.; Bucklel D R, Hedgecock C J R. Drug targets in inflammation and immunomodulation. Drug Discovery Today 2: 325-332, 1997; and, Kulkarni R G, Achaiah G, Sastry G N. Novel targets for antiinflammatory and antiarthritic agents. Curr Pharm Des. 2006; 12(19):2437-54.

The constructs of this aspect of the invention may include any combination of one or more peptides of the invention and one or more targeting molecule.

The peptides and constructs of this embodiment of the invention may be made using the techniques and containing additional components, including for example the elements described elsewhere herein for other constructs of the invention. For example, they may include additional heterologous molecules and linkers, including for example, those previously described herein. They may also include molecules that allow the construct to specifically target a desired cell. For example, where one may wish to target melanoma cells, a peptide comprising or consisting of YIGSR (a laminin pentapeptide) (SEQ ID No. 97) or Ac-Nle-Asp-His-D-Phe-Arg-Trp-Gly-Lys-NH2 (an α-melanocyte-stimulating hormone analogue) (SEQ ID No. 98) may be used. By way of further example, the constructs may also include additional motifs or molecules which may assist with cell permeability, as described previously for other constructs of the invention. In one particular embodiment, the construct includes a poly arginine peptide (for example R₄ (SEQ ID NO: 143) to Rₙ). By way of example, in one embodiment (relevant to targeting of B-raf) the construct includes R₈ (SEQ ID NO: 144) and comprises the amino acid sequence:

(SEQ ID NO 19)
RRRRRRRRHKLVRSPAPCKFFTSAGGLNVTAPTPQQLQAFKNEVGVLRK.

### Nucleic Acids Encoding Constructs and Nucleic Acid Vectors

Constructs of the invention may be produced using recombinant cloning and expression techniques and accordingly the invention should be taken to include nucleic acids encoding the constructs and vectors comprising such nucleic acids. In addition, it should be appreciated that nucleic acids encoding peptides/constructs of the invention could be used therapeutically or in vitro. For example, in an embodiment of the invention in which a peptide or construct of the invention is used in the treatment of HBV infection or HCC, or for the treatment of any other disorder, or to target the X-protein or any other protein for degradation, a nucleic acid/expression vector encoding the construct could be administered, with the peptide/construct subsequently being expressed. By way of further example, in the embodiment where peptides of the invention are used as carriers, the nucleic acid/expression vector could be administered to a subject, with the peptide/construct subsequently being expressed and delivered to a relevant cell or tissue. Accordingly, the invention includes nucleic acids and nucleic acid vectors suitable for these purposes.

Skilled persons will readily appreciate the sequence of nucleic acids encoding the constructs of the invention having regard to the nucleic acid and peptide sequences of the carrier peptides described herein before and the nature of the peptide compound to be delivered to a cell. Similarly, skilled persons will readily appreciate appropriate vectors for cloning and expressing the constructs. However, by way of example, the nucleic acid vectors (for example, pUC vectors, adeno-associated virus, lentivirus) and techniques detailed elsewhere herein (including those described in WO 91/09958, WO 03/064459, WO 00/29427, and WO 01/13957 for example) may be used.

### Compositions

The invention also provides compositions comprising the peptides and/or constructs (or nucleic acids encoding the peptides and/or constructs) of the invention in association with one or more diluents, carriers and/or excipients and/or additional ingredients. To this extent, it should be appreciated that reference herein to delivery or administration of a peptide, construct or nucleic acid of the invention is to include reference to delivery or administration of a composition comprising a peptide, construct and/or nucleic acid of the invention.

In one embodiment, the one or more diluents, carriers and/or excipients are suitable for use in vitro. In another embodiment, the one or more diluents, carriers and/or excipients are suitable for use in vivo (in this instance they may be referred to as “pharmaceutically acceptable”).

“Pharmaceutically acceptable diluents, carriers and/or excipients” is intended to include substances that are useful in preparing a pharmaceutical composition, may be co-administered with a peptide, construct, or nucleic acid encoding a peptide or construct of the invention while allowing it to perform its intended function, and are generally safe, non-toxic and neither biologically nor otherwise undesirable. Pharmaceutically acceptable diluents, carriers and/or excipients include those suitable for veterinary use as well as human pharmaceutical use. Examples of pharmaceutically acceptable diluents, carriers and/or excipients include solutions, solvents, dispersion media, delay agents, emulsions and the like.

In addition to standard diluents, carriers and/or excipients, a composition in accordance with the invention may be formulated with one or more additional constituents, or in such a manner, so as to enhance the activity of a peptide, construct, nucleic acid encoding a peptide or construct, and/or compound to be delivered to a cell, help protect the integrity or increase the half-life or shelf life of such agents, or provide other desirable benefits, for example. By way of example, the composition may further comprise constituents which provide protection against proteolytic degradation, enhance bioavailability, decrease antigenicity, or enable slow release upon administration to a subject. For example, slow release vehicles include macromers, polyethylene glycol), hyaluronic acid, poly(vinylpyrrolidone), or a hydrogel. By way of further example, the compositions may also include preserving agents, solubilising agents, stabilising agents, wetting agents, emulsifying agents, sweetening agents, colouring agents, flavouring agents, coating agents, buffers and the like. Those of skill in the art to which the invention relates will readily identify further additives which may be desirable for a particular purpose.

Furthermore, while not necessary for the performance of the invention, cell permeability of the peptides, constructs, nucleic acids encoding the peptides or constructs and/or compounds of the invention may be increased, or facilitated, through formulation of the composition. For example, the peptides, constructs, nucleic acids encoding the peptides or constructs and/or compounds of the invention may be formulated into liposomes. Further examples are provided in WO 91/09958, WO 03/064459, WO 00/29427, and WO 01/13957.

Additionally, it is contemplated that a pharmaceutical composition in accordance with the invention may be formulated with additional active ingredients which may be of benefit to a cell or a subject in particular instances. Persons of ordinary skill in the art to which the invention relates will readily appreciate suitable additional active ingredients having regard to the description of the invention herein and the purposes for which the delivery of the peptide, compound and/or construct is required, including, for example, the nature and progression of any disease to be treated. As a general example, agents used to treat HBV infection (interferon-α, Lamivudine, Adefovir dipivoxil (Hepsera), Baraclude (Entecavir)), or to prevent or inhibit the development of HCC (Sorafenib, Aurora Kinase Inhibitor PHA-739358, lactoferrin, omega 3 fatty acids, Gefitinib an EGFR inhibitor, Urocortin, angiogenesis inhibitors (eg TNP-470), Phenyl N-tert-butyl nitrone, immunostimulants) may be used

Compositions of the invention may be formulated into any customary form such as solutions, orally administrable liquids, injectable liquids, tablets, coated tablets, capsules, pills, granules, suppositories, trans-dermal patches, suspensions, emulsions, sustained release formulations, gels, aerosols, and powders, for example. Additionally, sustained release formulations may be utilised. The form chosen will reflect the purpose for which the composition is intended and the mode of delivery or administration to a sample or a subject. By way of example only, the dosage forms exemplified in WO 91/09958, WO 03/064459, WO 00/29427, and WO 01/13957 may be used where the compositions are formulated for administration to a subject, for example for the treatment of a disease (including but not limited to HBV infection and HCC).

Skilled persons will readily recognise appropriate formulation methods. However, by way of example, certain methods of formulating compositions may be found in Gennaro A R: Remington: The Science and Practice of Pharmacy, 20th ed., Lippincott, Williams & Wilkins, 2000.

### Methods

As mentioned herein before, in one embodiment, the invention provides methods of delivering one or more compounds to a cell using a carrier peptide or construct of the invention.

In another embodiment, the invention provides a method for targeting delivery of a compound to adherent cells in a mixed population of adherent and non-adherent cells. “Adherent” cells refers to are those cells attached to a substrate, matrix, other cells or surface. “Non-adherent” cells refers to cells that are not attached to a substrate, matrix, other cells, or surface (for example, circulating cells in the blood).

In another embodiment, the invention provides methods of antagonising or disrupting the function of an X-protein using certain peptides described herein (including functionally equivalent variants thereof) or constructs comprising such peptides or functionally equivalent variants. In one particular embodiment, the invention provides a method for the treatment of HBV infection, the method comprising administering such peptide (including functionally equivalent variants thereof) or constructs comprising such peptides or functionally equivalent variants to a subject. In another particular embodiment, the invention provides a method for the treatment (including prevention) of HCC, the method comprising administering such peptide (including functionally equivalent variants thereof) or constructs comprising such peptides or functionally equivalent variants to a subject. It should be appreciated that such methods may also comprise administering to a subject a nucleic acid encoding relevant peptides or constructs of the invention and/or a vector comprising such nucleic acid.

In another embodiment, the invention provides methods for targeting a protein for subsequent degradation using certain peptides described herein (including functionally equivalent variants) or constructs comprising such peptides or functionally equivalent variants. It should be appreciated that such methods may also comprise use of a nucleic acid encoding relevant peptides or constructs of the invention and/or a vector comprising such nucleic acid. Such methods may be used for research purposes or in the treatment of disease, for example a disease associated with an undesirable level or activity of one or more proteins, or for example for knockdown of proteins in cells and tissues for research purposes. By way of example, the method may be used to treat cancers by targeting one or more specific proteins (as may be exemplified herein). In one particular embodiment, the method is for the treatment of melanoma, by targeting one or more of B-raf, N-ras and The PDGF receptor for degradation. Further examples of suitable targets for the treatment of cancers and inflammatory disorders are provided herein before. However, by way of specific example, the method is for the treatment of cancer by targeting one or more of the proteins CD20, HER2 (ErbB-2), phospholipase C-gamma, c-Met, c-myc, insulin-like growth factor, ras, raf, mitogen-activated protein kinase (MEK), phosphatidylinositol 3-kinase (PI-3 kinase), 3-phosphoinositide-dependent protein kinase (PDK), mammalian target of rapamycin (mTQR), akt kinase, src, (histone deacetylase) HDAC, Bcl-2, XIAP, hsp90, Flt3, c-kit, cyclin-dependent kinase, lysophosphatidic acid (LPA) receptor, autotaxin, CD33, CD52, EGFR, VEGF, VEGF receptor, hypoxia-inducible factor, Delta-like 4, survivin, LMTK3, RANK ligand, indoleamine 2,3-dioxygenase (IDO), O-GlcNAc transferase, matrix metalloproteinase, Bcr-Abl, anaplastic lymphoma kinase, oestrogen receptor, aromatase, androgen receptor, poly ADP ribose polymerase, integrins, CTLA-4, and the molecules with which all the latter molecules interact.

Delivery of the peptides and/or constructs (or nucleic acids encoding same) of the invention may occur in vivo or in vitro, depending on the purposes for which delivery is required.

The peptides and/or constructs (or nucleic acids or vectors encoding same) may be delivered to a cell by a number of different means, as will be readily appreciated by persons skilled in the art. However, by way of example, an in vitro method may comprise bringing the construct and/or peptide (or nucleic acids or vectors encoding same) into contact with one or more cells or a composition comprising one or more cells or proteins of interest (for example, in embodiments relating to antagonising X-protein function, into contact with one or more cells and/or one or more X-proteins or a composition comprising one or more cells and/or one or more X-proteins); for example, contacting the construct or peptide (or nucleic acids or vectors encoding same) with a sample, composition or media in which the one or more cells (or proteins of interest in certain embodiments) are contained (such as mixing a composition of the invention with a liquid sample containing one or more cells or proteins). In another embodiment, a method of the invention comprises administering a construct and/or peptide (or nucleic acids or vectors encoding same) to a subject.

It will be appreciated by those of general skill in the art to which the invention relates, having regard to the nature of the invention and the results reported herein, that the present invention is applicable to a variety of different animals. Accordingly, a “subject” includes any animal of interest. However, in one particular embodiment the “subject” is a mammal, more particularly human.

As mentioned elsewhere herein, the inventors have identified that the carrier peptides and related constructs of the invention comprising the carrier peptides are not taken into non-adherent cells. The inventors contemplate that this makes such peptides and constructs more suitable for systemic administration than other known peptides and constructs as the dose administered should not be diluted by non-specific uptake of the agents into circulating blood cells. However, administration to a subject may occur by any means capable of delivering the agents of the invention (peptides, compounds, constructs or nucleic acids or vectors encoding same) to target cells within the body of a subject. By way of example, agents of the invention may be administered by one of the following routes: oral, topical, systemic (eg. transdermal, intranasal, or by suppository), parenteral (eg. intramuscular, subcutaneous, or intravenous injection), by administration to the CNS (eg. by intraspinal or intracisternal injection), by administration to the liver (eg by intraportal injection), by implantation, and by infusion through such devices as osmotic pumps, transdermal patches, and the like. Skilled persons may identify other appropriate administration routes. Exemplary administration routes are also outlined in WO 91/09958, WO 03/064459, WO 00/29427, and WO 01/13957 for example.

As will be appreciated, the dose of an agent administered, the period of administration, and the general administration regime may differ between subjects depending on such variables as the reason for delivery of the agent, the target cells to which the agent is to be delivered, and the severity of any symptoms of a subject to be treated, the type of disorder to be treated, the mode of administration chosen, and the age, sex and/or general health of a subject.

It should be appreciated that administration may include a single daily dose, administration of a number of discrete divided doses, or continuous administration as may be appropriate.

Data obtained from cell culture assays and animal studies can be used in formulating a range of dosages for use in humans. The dosage may vary within this range depending upon the dosage form employed and the route of administration utilized. For any agent used in the method of the invention, the therapeutically effective dose can be estimated initially from cell culture assays. A dose may be formulated in cell cultures or animal models to achieve a cellular concentration range that includes the IC50 (i.e., the concentration of the test compound that achieves a half-maximal inhibition of symptoms) as determined in cell culture. Such information can be used to more accurately determine useful doses in humans. The exact formulation, route of administration and dosage can be chosen by the individual physician in view of the patient's condition. (See, e.g., Fingl et al., 1975, In: The Pharmacological Basis of Therapeutics, Ch. 1, p. 1).

Administration could occur at any time during the progression of an HBV infection, or prior to or after the development of HCC, including from the time of a suspected infection. In one embodiment, the agents of the invention are administered on a daily basis for an extended period to assist with ongoing management of viral load and symptoms. In another embodiment, the agents of the invention are administered on a daily basis for an extended period or life-long to prevent or delay the development of HCC. Additional examples of administration regimes are provided in WO 91/09958, WO 03/064459, WO 00/29427, and WO 01/13957.

It should be appreciated that a method of the invention may further comprise additional steps such as the delivery of additional agents or compositions to a sample, cell or subject.

### EXAMPLES

### Example 1

### Materials and Methods

### Peptides

Peptides were ordered from Peptide 2.0, 14100 Sullyfield Circle, Suite 200, Chantilly, Va. 20151, USA.

TABLE 3

| Corresponding | | Molecular | SEQ |
|---|---|---|---|
| Hbx Regin | Peptide sequence | weight | ID NO |
| 1-20 | FITC-Acp-MAARLCCQLDPARDVLCLRP | 2747 | 3 |
| 16-35 | FITC-Acp-LCLRPVGAESRGRPVSGPFG | 2558 | 6 |
| 21-40 | FITC-Acp-VGAESRGRPVSGPFGPLSSP | 2457 | 84 |
| 34-53 | FITC-Acp-FGPLSSPAFSVPADHGAHLS | 2497 | 85 |
| 1-50 | Biotin-MAARLCCQLDPARDVLCLRPVGAESRGR | 5360 | 86 |
| | PVSGPFGPLSSPAFSVPADHGA | | |
| 16-26 | FITC-LCLRPVGAESR | 1703 | 5 |
| 16-24 | FITC-LCLRPVGAE | 1406 | 4 |
| 16-22 | FITC-LCLRPVG | 1260 | 2 |
| 16-20 | FITC-LCLRP | 1102 | 1 |
| 16-35 with HIF-1 | FITC-Acp- | 4838 | 78 |
| ODD | RRRRRRRRMLAPYIPMGGLCLRPVGAESRGRPVSG | | |
| | PFG | | |
| 16-35 with HIF-1 | FITC-Acp-RRRRRRRRLCLRPVGAESRGRPVSGP | 4781 | 79 |
| ODD | FGGMLAPYIPM | | |
| 16-35 with X- | Biotin- | 5418 | 80 |
| protein instability | RRR_RRRRRHKLVRSPAPCKFFTSAGGLCLRPVGA | | |
| domain | ESRGRPVSGPFG | | |
| 16-35 with X- | Biotin- | 5621 | 81 |
| protein instability | RRRRRRRRLCLRPVGAESRGRPVSGPFGGCRHKLV | | |
| domain | RSPAPCKFFTSA | | |

### Mammalian Cell Lines

The HepG2 cell line is a liver cancer cell line derived from a male human patient with hepatocellular carcinoma. The cell line was sourced from the ATCC (cat # HB-8065). There is no evidence of the hepatitis B virus genome in this cell line. The cells were propagated in full MEM medium at 37° and 5% CO₂ from cells that had been frozen in a freezing solution containing full MEM and 5% DMSO.

### Purification of Plasmid DNA

Three plasmids were used that had been propagated in DH5α E. coli cells. The plasmid pCDNA3.1-HBX encodes a truncated form of the X-protein lacking the instability domain (aa 141-153). It was prepared in-house from a series of 8 overlapping oligonucleotides, and the sequence confirmed by DNA sequencing, as below:

Truncated X-protein sequence with flanking restriction sites encoded by pCDNA3.1-HBX. HindIII and KpnI sites at the 5′-end and an Xba I restriction site at the 3′-end.

(SEQ ID NO 87)
5′ GGG AAG CTT GGT ACC

[symbol]

[symbol]

[symbol]

[symbol]

[symbol]

[symbol]

[symbol]

[symbol]

[symbol]

[symbol]

TAG CTC TAG TCT AGA GC 3′
(SEQ ID NO 88)
5′ GCTCTAGACTAAGCTA

[symbol]

[symbol]

[symbol]

[symbol]

[symbol]

[symbol]

[symbol]

[symbol]

[symbol]

GGTACCAAGCTTCCC 3′

The plasmid pCDNA3-GFP expresses green fluorescent protein. The plasmid pCDNA3-HBX Myc encodes the full-length X-protein and was kindly donated by Professor Massimo Levrero, Department of Internal Medicine, Universita di Cagliari).

Starter cultures containing bacteria transformed with each of the above three plasmids were created by adding 5 μl of each transformant from frozen stocks into 10 ml of LB containing 100 μg/ml of ampicillin. The cultures were placed on a shaker at 37° overnight. The starter culture was then used to make a larger batch culture. The 10 ml starter culture was combined with 400 ml of LB containing 100 μg/ml of ampicillin. The culture was placed on a shaker at 37° overnight. A maxiprep plasmid purification kit (Qiagen, Invitrogen) was then used to purify the plasmid DNA from the culture. Cultures were centrifuged at 6,000 g or 10 min, and plasmid DNA extracted from the bacterial pellet using the Qiagen maxiprep kit according to the manufacturers' instructions. The extracted DNA samples were centrifuged at 20,000 g for 30 min, and the supernatant collected and recentrifuged at 18,000 g for 15 min. The resulting supernatant was poured through elution tubes containing anion-exchange resin to capture the DNA. The DNA was then eluted with elution buffer and the recovered DNA was then precipitated by adding 10.5 ml of isopropanol to 15 ml of DNA sample, followed by centrifugation at 20,000 g for 30 min. The DNA pellet was then resuspended in dH₂O. The concentration of DNA in the samples was measured with a nano-drop spectrophometer (Nanodrop ND-1000).

### Pretreatment of Plasmid DNA Prior to Transfection

Before plasmid DNA was used in transfection it was sterilized to prevent contamination of the HepG2 cells. To the DNA suspended in dH₂O was added NaOAc to 0.3 M and 100% ETOH to 30% volume, and the solution was placed on ice for 10 min. The DNA was precipitated by centrifugation at 13,000 rpm for 10 min, and washed with 70% (v/v) cold EtOH. Traces of EtOH were removed in a speed-vac, and the DNA was resuspended in sterile dH₂O. All samples were resuspended to a final concentration of 1 μg/μl of DNA. DNA yield was measured on a nano-drop spectrophometer (Nanodrop ND-1000).

Treatment of HepG2 Transfectants with X-Protein-Degradation Domain Fusion Peptides

HepG2 cells were seeded into 6 well plates at 1×10⁶ cells per well in MEM medium supplemented with 10% FCS, and cultured overnight at 37° and 5% CO₂. No PSG antibiotic was added to the medium as it can interfere with transfection efficiency. The medium was then removed and replaced with MEM medium without 10% FCS. The amount of DNA used for transfection was 4 μg. Firstly, 4 μg of DNA was mixed with 250 μl of Opti-MEM medium, and then 4 μl of polymag transfection reagent was added. The mixture was incubated for 20 min and then added to the cells. The plate was placed onto a magnetic plate on a rotating platform for 20 min.

Cell-permeable X-protein-degradation domain fusion peptides were added at a concentration of 10 μM to the cells in MEM without 10% FCS and cells incubated for 45 h. Peptides were added to MEM without FCS as serum can rapidly degrade small peptides. An additional 10 μM of peptide was added after 3 h of incubation, and a further 10 μM of peptide was added after 21 h incubation. When the additional aliquots of peptide were added, the cells were washed in MEM without FCS, the peptides added, and the cells incubated for 3 h before changing the medium to MEM containing 10% FCS and PSG. The cells were lysed and analyzed by Western blot analysis for expression of the X-proteins.

Treatment of HepG2 Transfectants with the Oligomerization Domain Peptide

HepG2 cells were seeded into 8-well chamber slides at 1×10⁵ cells per well in MEM medium supplemented with 10% FCS, and cultured for 24 h at 37° and 5% CO₂. No PSG antibiotic was added to the medium as it can interfere with transfection efficiency. The medium was then removed and replaced with opti-MEM medium without 10% FCS. The amount of plasmid DNA used for transfection was 0.1 μg. Firstly, 0.2 μg of either pcDNA3.1-HBX or pcDNA3-HBX Myc plasmid DNA was mixed with 25 μl of Opti-MEM medium, and then 0.2 μl of polymag transfection reagent was added (these solutions were prepared as a master mixes—the amounts mentioned are indicative for one well). The mixture was incubated for 20 min and then 25 μl added to the cells. The plate was placed onto a magnetic plate on a rotating platform for 20 min, and then the cells incubated for 3 h.

Cells were transfected with X-protein plasmid vectors as described above. After 3 h of transfection on the magnetic plate, the oligomerization domain peptide aa 16-35 or the control X-protein peptide aa 140-153 were added at a final concentration of 10 μM to the cells in MEM without 10% FCS, and cells incubated for either 3 h, 21 h, or 45 h. Peptides were added to MEM without FCS as serum can rapidly degrade small peptides. For cells incubated for 21 h, an additional 10 μM of peptide was added after the 3 h incubation. For cells incubated for 45 h with peptide, an additional 10 μM of peptide was added after the 3 h incubation, and a further 10 μM of peptide was added after the 21 h incubation. When the additional aliquots of peptide were added, the cells were washed in MEM without FCS, the peptides added, and the cells incubated for 3 h before changing the medium to MEM containing 10% FCS and PSG. In summary, the peptides were added to the cells at either 3 h, 3 and 24 h, or 3, 24, and 48 h post transfection, and the cells were cultured for a total of 51 h.

Annexin-V labelling solution was prepared by adding 20 μl of Annexin V labelling reagent and 10 μl of propidium iodide to 1 ml of incubation buffer. The cells were washed with incubation buffer, and 100 μl of Annexin-V labelling solution was added to each well, followed by incubation for 15 min in the dark at room temperature. The cells were washed with incubation buffer, fixed with 2% formaldehyde in PBS for 30 min, washed with PBS, and the slide chambers removed. A drop of mounting solution with DAPI was added to the cells, and covered with a cover slip. Slides were examined by fluorescence microscopy by using a Nikon E600 fluorescent microscope and photos were taken using Nikon ACT-1 software.

### Cell Lysis

Cells were washed with PBS, and detached from plates by incubation with trypsin diluted in PBS. Cells were washed with MEM containing FCS to inactivate trypsin, and pelleted by centrifugation at 175 g for 5 min. They were then resuspended in 1 ml of PBS, and a 10 μl sample was removed, stained with Trypan blue, and cell number counted with a haemocytometer. The cells were then pelleted at 4,000 g for 5 min and thrice washed in PBS. All traces of liquid were removed and the cells were ready for cell lysis. Complete Lysis Buffer was added to the cells at 20 μl per 1×10⁶ cells. The cells were resuspended very well to aid lysis and then left on ice for 10 min. They were centrifuged at 15,000 g for 10 min, and the supernatant collected and saved. The nuclear precipitate was processed by adding 13 μl of lysis buffer, 2 μl of 2% SDS, 2 μl of Na deoxycholate, 2 μl of DNAse buffer and 1 μl of DNAse I. The sticky cell lysate was resuspended vigorously after each reagent was added then incubated at 37° for 10 min.

### Peptide Binding Assay

Qligomerization peptides were received in powdered form, and were weighed and diluted in a sterile 10% DMSO solution to make a 10 mg/ml stock solution. The wells of a Reactibind neutravidin-coated 96 well plate (Thermoscientific cat #15507) were thrice washed with 200 μl of plate washing buffer. Peptide 1-50, which contains a biotin tag, was diluted to 10 μg/ml in wash buffer and 100 μl of the solution was placed into each well being used. The plate was incubated at room temperature for 2 h, and the wells washed thrice with 200 μl of wash buffer. Truncated fluorescent variants of peptide 1-50 at 40 μg/ml, 20 μg/ml, and 10 μg/ml were added to the wells in triplicate, and the plates incubated at room temperature for 30 min. The wells were then washed thrice with 200 μl of wash buffer under low light conditions, and the plate read on a Biotek fluorescent plate reader at an excitation wavelength of 490 nm and an emission wavelength of 520 nm.

### Assay for Cell Permeability of X-Protein Oligomerization Peptides

Cells were plated into 8-well chamber slides at 1×10⁵ cells per well in MEM with 10% FCS and PSG. The cells were then incubated overnight at 37° and 5% CO₂, and then washed thrice with media without FCS. The X-protein peptide in 250 μl of appropriate media without FCS was added to the cells at a final concentration of 10 μM, and the slides incubated for 3 h at 37° C. and 5% CO₂. The cells were washed with PBS, fixed with 4% formaldahyde in PBS for 30 min, and washed thrice with PBS. The plastic frame was removed from the slides, and a drop of Prolong Gold anti-fade reagent with DAPI (Invitrogen cat# P36931) was added to each sample. The slides were dried overnight in the dark, and examined by microscopy by using a Nikon E600 fluorescent microscope. Photos were taken using Nikon ACT-1 software. For further examination, slides were then examined by confocal microscopy using a Leica TCS-SP2 confocal microscope.

Elucidating the Pathway by which the Cell-Permeable X-Protein Peptide Enters Cells

Cells were plated overnight at 1×10⁵ per well in eight chamber slides in complete MEM medium containing 10% FCS. The cells were washed thrice with MEM medium, and pretreated for 30 min with either 10 μm cytochalasin D or 2 μg/ml of heparin in MEM at 37° C. Peptide was then added at a concentration of 10 μM and cells were incubated for 3 h at 37° and 5% CO₂. The cells were washed with PBS, fixed with 4% formaldehyde and PBS for 30 min, and a drop of Prolong Gold antifade reagent with DAPI (Invitrogen cat# P36931) was added to each well. The slides were dried overnight in the dark, and examined by microscopy by using a Nikon E600 fluorescent microscope. Photos were taken using Nikon ACT-1 software.

Carriage of a Foreign Peptide into TK-1 Cells by the X-Protein Cell-Permeable Peptide aa 16-22

Eight-well glass chamber slides were coated overnight at 4° C. with MAdCAM-1-Fc. They were blocked with FCS at RT for 2 h. TK-1 T cells were activated with 2 mM Mn2+/Ca2+ in HBSS buffer and added to MAdCAM-1 coated glass slides, followed by incubation for 15 min. Peptides LCLRPVGGYDRREY (SEQ ID NO 15) and R9YDRREY (SEQ ID NO 14) were added at 50 μM followed by incubation for 30 min. The cells were fixed overnight in 4% PFA, permeabilised with PBS+0.1% Tween 20 for 15 min, blocked with 3% BSA in PBS for 1 h, and the presence of the peptides detected with FITC-Streptavidin (1:200 dilution in 3% BSA in PBS 1 h). Slides were mounted with Prolong gold with DAPI.

### Results

### Engineered Expression of the X-Protein in HepG2 Cells

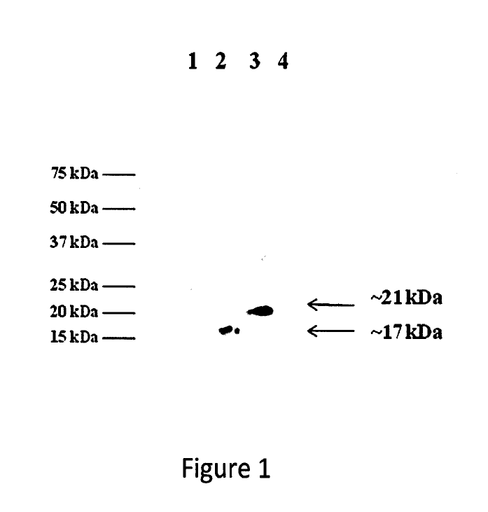

HepG2 cells were transfected with 4 μg of the X-protein plasmid pCDNA3.1-HBX, pCDNA3-HBX Myc or 4 μg of the pCDNA3-GFP vector and cultured for 48 h. Preliminary experiments had shown that 4 μg of plasmid DNA was the optimum amount of plasmid, as higher amounts did not produce higher levels of X-protein. Cells were transfected with the pCDNA3-GFP vector to visualize transfection efficiency as the X-protein vectors did not possess markers. Transfection efficiency 24 hours post-transfection was determined to be 20-30% (data not shown). This level of transfection was similar to that seen at 48 hours post-transfection, though the level of fluorescence emitted by each cell was visibly higher indicating higher expression of the GFP protein by each cell. Transfection of cells for 48 h was used for the remainder of the study as the X-protein was only detected by Western blot analysis after 48 h of transfection. Transfectants were subjected to Western blot analysis to determine whether they expressed the X-protein. In addition, normal HepG2 cells that had not been transfected were used as an additional control to confirm that the X-protein was not endogenously expressed in the HepG2 cell line. The cells were lysed, and the lysates subfractionated into the supernatant which contained the detergent soluble proteins, and the precipitate which contained the cell nuclei and detergent-insoluble material. The supernatant and nuclear subfractions were resolved by SUS-PAGE, and the proteins transferred onto a PVDF membrane. The membrane was probed with a mouse anti-human X-protein primary antibody, followed by a secondary goat anti-mouse HRP-conjugated antibody. The anti-X-protein antibody recognized two distinct bands in the lanes containing the nuclear extracts of HepG2 cells transfected with the pCDNA3.1-HBX and pCDNA3-HBX Myc plasmids (FIG. 1). The bands were not present in the detergent soluble fraction of HepG2 cells. The sizes of the bands correlated with the expected sizes of the plasmid-encoded proteins. The protein encoded by the pCDNA3.1-HBX plasmid is a truncated ˜17 kDa version of the X-protein which is missing the instability domain encompassing aa 141-153. In contrast, the protein encoded by pCDNA3-HBX-Myc is the full-length X-protein to which a myc tag has been added, giving a total size of ˜21 kDa. The X-protein was not expressed in HepG2 cells that had not been transfected or in HepG2 cells that were transfected with pCDNA3-GFP only.

### Defining the Minimum Peptide Motif Needed for Oligomerization of the X-Protein

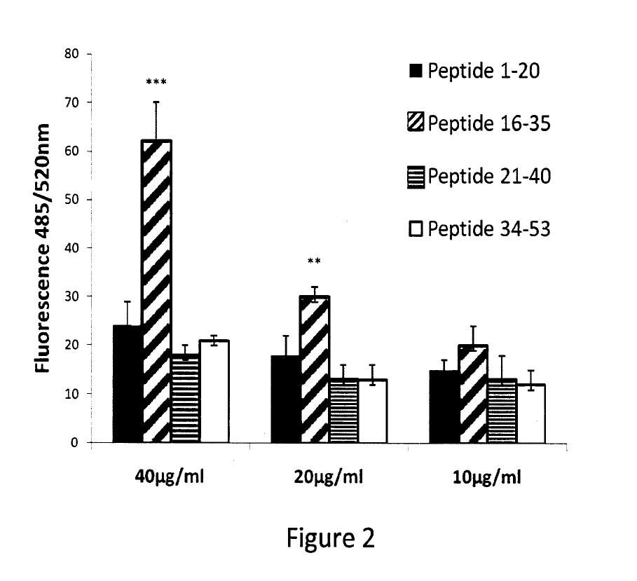

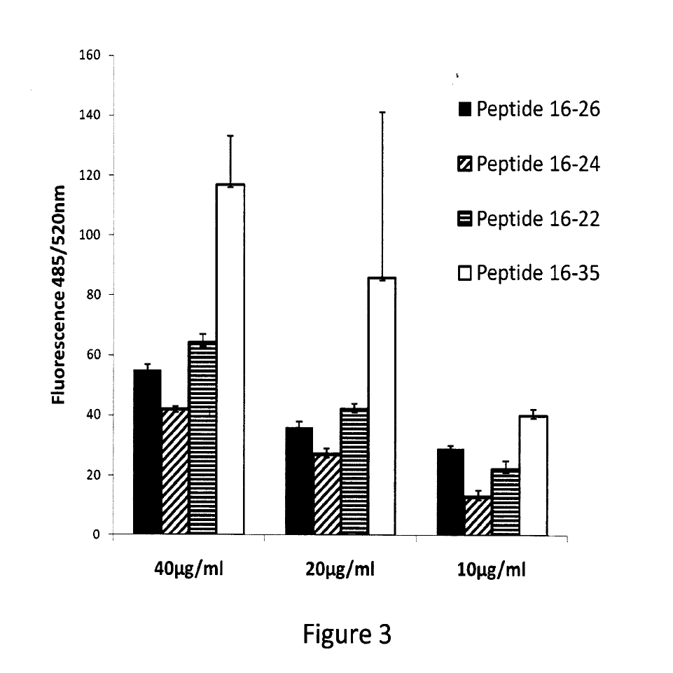

A ligand-binding assay was performed using streptavidin-coated plates in order to define the minimum peptide motif needed for dimerization of the X-protein. In this scenario the X-protein monomer serves as its own ligand. The oligomerization domain for the X-protein has previously been shown to be contained within the N-terminal 50 aa residues. A series of short peptides from the region encoding aa 1-50 were synthesized. The parent peptide containing aa 1-50 conjugated N-terminally to a biotin tag was added to the streptavidin coated plates. Streptavidin displays high affinity binding to biotin so was able to capture and secure the peptide to the plate. Non-biotinylated peptides encoding aa 1-20, 16-35, 21-40 and 34-53 were added at 10, 20, and 40 μg/ml to the wells coated with peptide aa 1-50. The smaller peptides were each conjugated to a FITC tag for detection using a fluorescent plate reader. Peptide 16-35 bound significantly more strongly to peptide 1-50 at 20 and 40 μg/ml than did, peptides 1-20, 21-40, and 34-53 (Table 4; FIG. 2). Binding performed with lesser concentrations of the peptides was not significant. Even smaller peptides encompassing that region 16-35 were made to determine whether the aa 16-35 was the minimum peptide motif needed for X-protein dimerization. These peptides synthesized, namely aa 16-26, 16-24, and 16-22 were analyzed using the same peptide binding assay. The peptides showed no difference in their ability to bind peptide 1-50, and could not match the strong binding shown by the parental peptide 16-35 (FIG. 3). Thus of the peptides examined, the minimal peptide motif needed for dimerization of the X-protein is the 30 aa peptide 16-35.

TABLE 4
Statistical significance of binding of X-protein peptides to peptide aa 1-50

| | P values | | |
|---|---|---|---|
| | 40 μg/ml | 20 μg/ml | 10 μg/ml |
| Peptide 1-20 | 0.05137 | 0.21476 | 0.49701 |
| Peptide 16-35 | 0.00076 | 0.00579 | 0.12771 |
| Peptide 21-40 | 0.19482 | 0.92516 | 0.93715 |
| Peptide 34-53 | 0.06399 | 0.9283 | 0.78122 |

### Creation of a Novel Peptide Capable of Degrading the X-Protein Based on the ODD of HIF-1

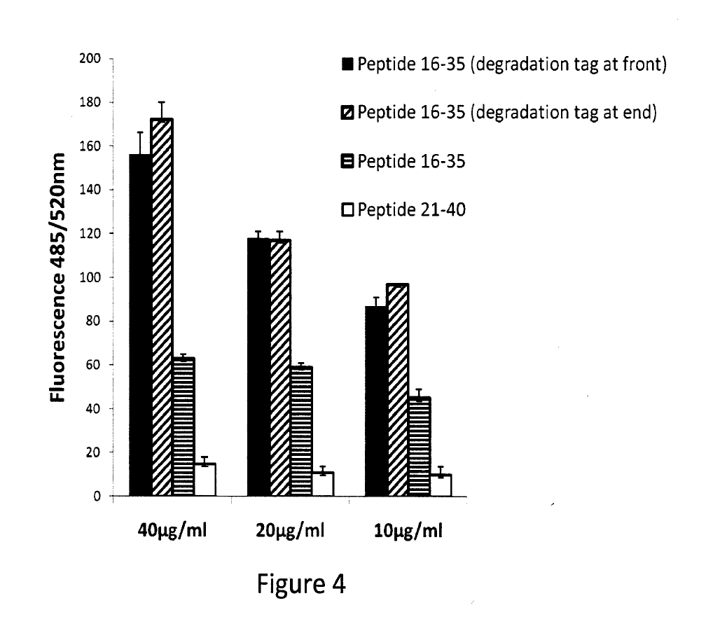

The above results established that the X-protein oligomerization domain encompasses aa 16-35. An approach was devised to exploit peptide 16-35 as a targeting peptide to which the oxygen-dependent degradation (ODD) domain of HIF-1α would be attached. Peptide 16-35 was fused N- and C-terminally during synthesis to the 8 aa ODD domain, and an R8 (SEQ ID NO: 144) sequence was added N-terminally to render the peptide cell-permeable. The notion was that the targeting X-protein-ODD fusion peptides would enter X-protein expressing cells, bind to the oligomerization domain of the X-protein and be recognized by pVHL, leading to ubiquination of the peptide and its degradation together with the attached X-protein. The X-protein-ODD fusion peptides were first tested to determine whether they were capable of binding to the oligomerization peptide 1-50. They were able to bind peptide 1-50, irrespective of whether peptide 16-35 was located at the front or end of the targeting peptide (FIG. 4). Surprisingly, they bound better to peptide 1-50 than did the parental peptide 16-35. The non-binding peptide 21-40 was used as a negative control, and again failed to appreciably bind peptide 1-50.

### X-Protein-ODD Fusion Peptides Decrease X-Protein Expression

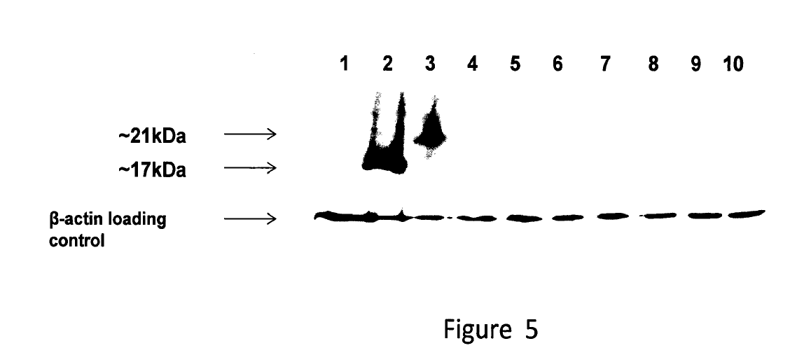

The two X-protein-ODD fusion peptides were tested for their ability to decrease the level of X-protein expression. HepG2 cells were transfected with 4 μg of either the X-protein plasmids pCDNA3.1-HBX and pCDNA3-HBX Myc, or 4 μg of the pCDNA3-GFP vector. The X-protein-ODD fusion peptides were repetitively added to the cells at 3 h, 24 h and 48 h post transfection. Control transfectants were not treated with peptide, but were otherwise cultured under the same conditions. Transfectants were subjected to Western blotting to determine whether the X-protein was expressed. The cells were lysed, and the lysates centrifuged to give a supernatant fraction which contained detergent-soluble proteins, and a precipitate which contained the cell nuclei and detergent-insoluble material. The supernatant and nuclear subfractions were resolved by SDS-PAGE, and the proteins transferred onto a PVDF membrane. The membrane was probed with a mouse anti-human X-protein primary antibody, followed by a secondary goat anti-mouse HRP-conjugated antibody. As above, the X-protein was not present in the detergent soluble fraction of HepG2 transfectants (FIG. 5). In contrast, it was present in the nuclear fraction of cells transfected with pCDNA3-HBX and pCDNA3-HBX Myc as a truncated protein of ˜17 kDa and a full-length protein of ˜21 kDa, respectively. The X-protein-ODD fusion peptide with the ODD tag at the front caused complete degradation of the X-protein produced by pCDNA3.1-HBX and pCDNA3-HBX Myc (FIG. 5). The X-protein-ODD fusion peptide with the ODD tag at the end caused near complete degradation of the X-protein (FIG. 5). The control cells transfected with pCDNA3.1-GFP and untransfected HepG2 cells gave no bands indicating that the mouse anti-human X-protein primary antibody specifically recognized the X-protein. The blot was probed with an antibody against β-actin which confirmed that each lane contained an equal amount of lysate.

### Creation of a Novel Peptide Capable of Degrading the X-Protein Based on the Instability Domain of the X-Protein

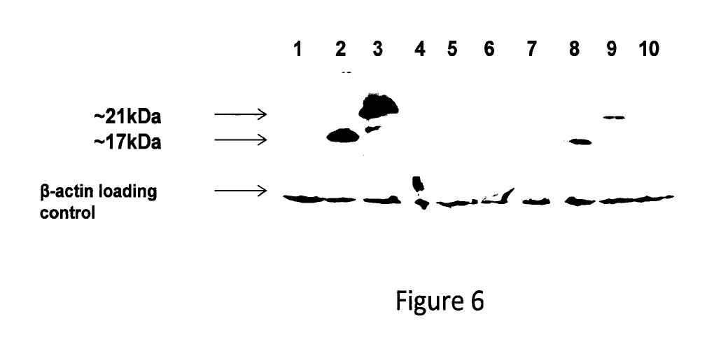

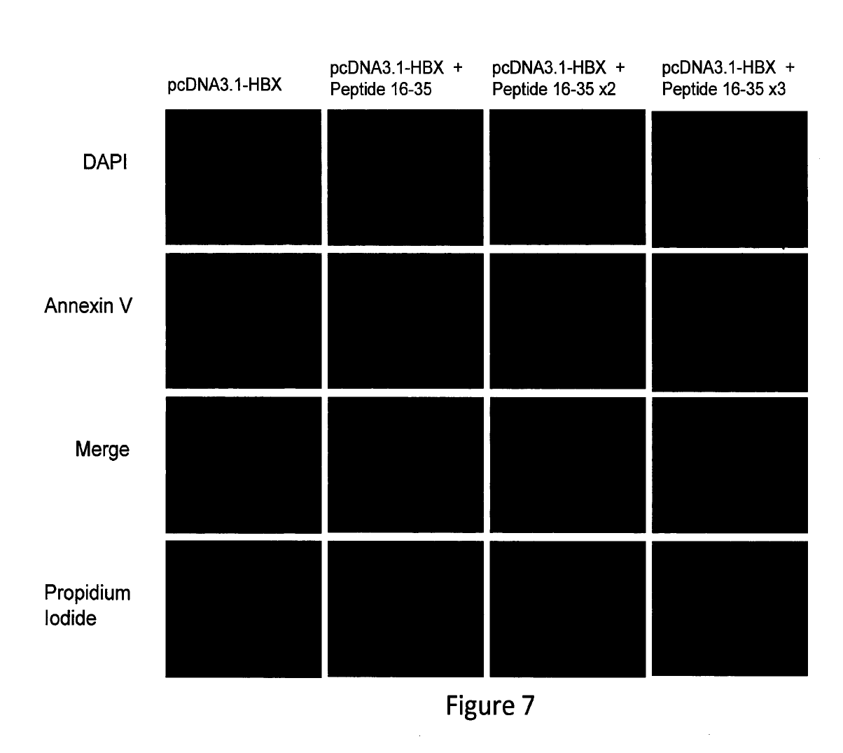

The X-protein contains a region which maps to aa 140-153 which is responsible for the stability of the protein (Li et al. 2006).²⁵ Cell-permeable peptides containing the instability domain (aa 140-153) fused to the X-protein oligomerization domain (aa 16-35) were used to investigate the effect of the instability domain on X-protein expression. Two fusion peptides were created with the instability domain attached either in front of the X-protein oligomerization domain or at the end. They were rendered cell-permeable with an N-terminal R8 (SEQ ID NO: 144) tag carrier peptide. The peptides were repetitively added to HepG2 cells 3 h, 24 h and 48 h post transfection with X-protein plasmids. Control transfectants were not treated with peptide, but were otherwise cultured under the same conditions. The nuclear fraction of transfectants were subjected to Western blotting to determine whether the X-protein was expressed. The anti-X-protein antibody recognized the truncated (˜17 kDa) and full-length (˜21 kDa) forms of the X-protein in cells transfected with pCDNA3.1-HBX and pCDNA3-HBX Myc, respectively (FIG. 6). The X-protein oligomerization-instability domain fusion peptide with the instability domain in front caused complete degradation of the X-protein produced by pCDNA3.1-HBX and pCDNA3-HBX Myc. The X-protein oligomerization-instability domain fusion peptide with the instability domain at the end caused near complete degradation of the X-protein, with slight preference for the full length protein (FIG. 7). Untransfected parental HepG2 cells and those transfected with pCDNA3.1-GFP showed no X-protein bands.

### A Stand-Alone Form of the Oligomerization Domain of the X-Protein Inhibits the Proapoptotic Function of the X-Protein

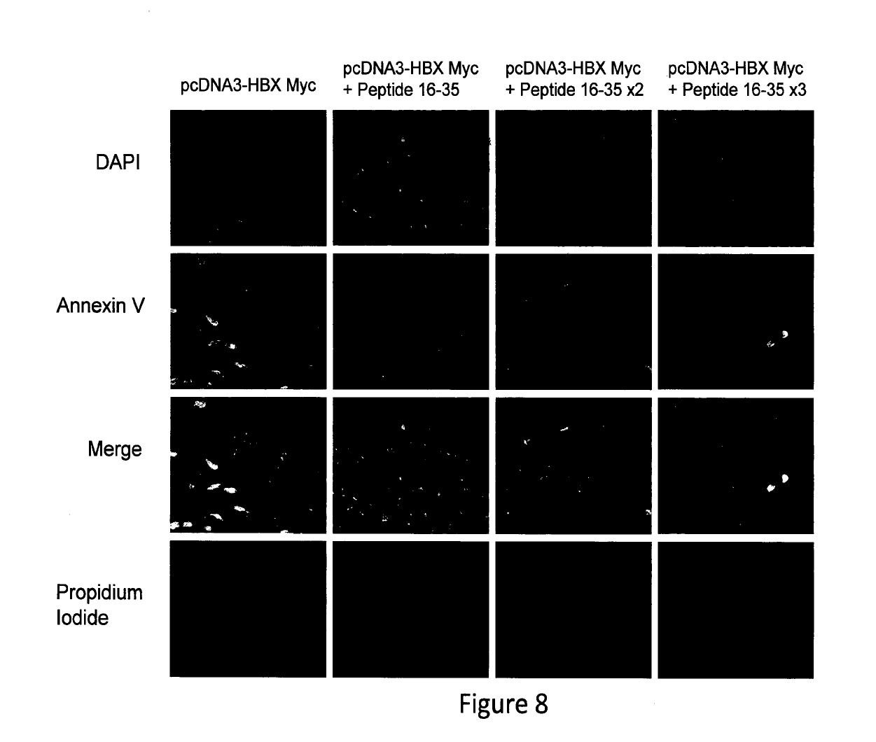

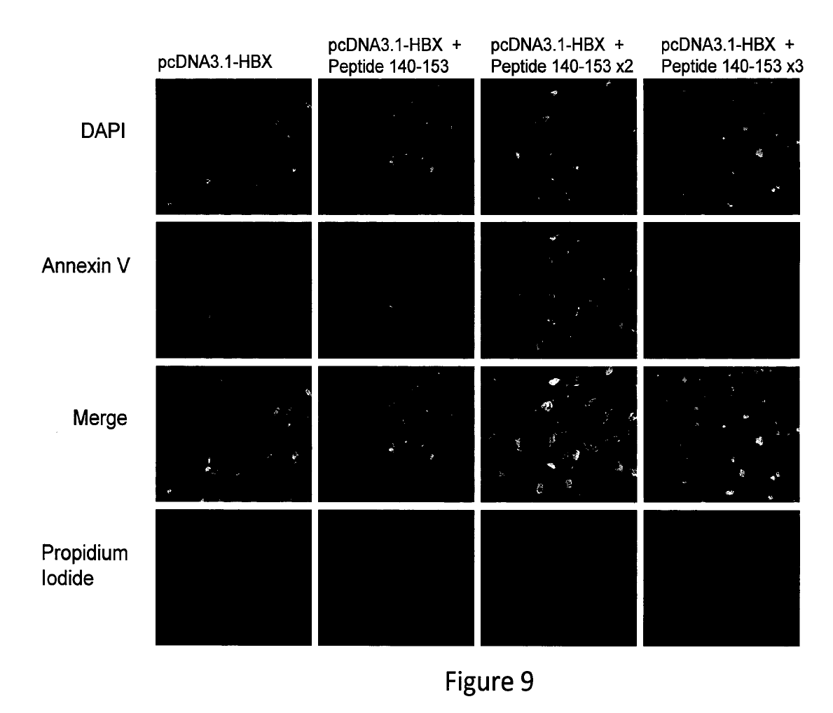

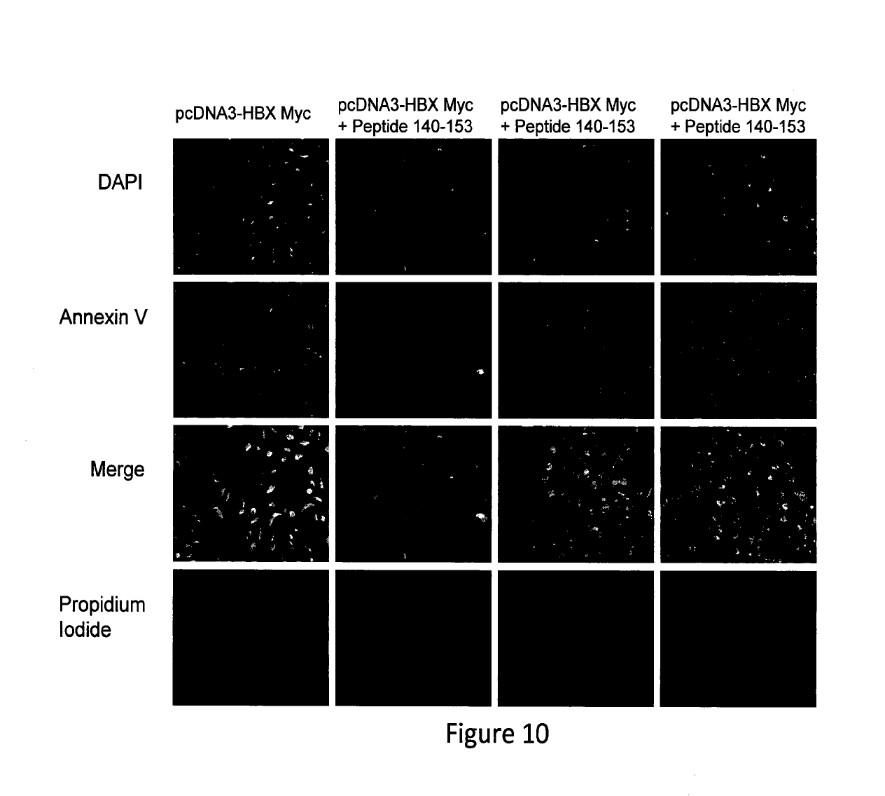

The oligomerization domain peptide (aa 16-35) was tested for its ability to inhibit the pro-apoptotic function of the X-protein. The hypothesis was that the peptide would inhibit dimerization of the X-protein which is a pre-requisite for function (Murakami et al, 1994).²⁶ HepG2 cells were engineered to express the X-protein by transfection with the pCDNA3.1-HBX and pCDNA3-HBX Myc plasmids. The X-protein oligomerization peptide (aa 16-35) was added to the cells at either 3 h, 3 and 24 h, or 3, 24, and 48 h post transfection, and the cells were cultured for a total of 51 h. Control cells were transfected with either pCDNA3.1-HBX or pCDNA3-HBX Myc and treated with a cell-permeable form of the X-protein peptide aa 140-153 which does not bind the X-protein. Cells were stained with annexin V to visualize the early stage of apoptosis, and counterstained with propidium iodide (PI) to detect necrosis. DAPI was added to stain the cell nuclei. Approximately 80 to 90% of the cells transfected to express either the truncated (FIG. 7) or full-length (FIG. 8) forms of the X-protein underwent apoptosis. No cell necrosis was seen over the time course examined. Addition of the oligomerization domain peptide (aa 16-35) significantly reduced X-protein-mediated apoptosis in a dose-dependent fashion (FIGS. 7 and 8). In contrast, apoptosis of transfectants was unaffected by X-protein peptide aa 140-153 which does not bind the X-protein (FIGS. 9 and 10). Thus, the ability to inhibit X-protein-mediated apoptosis is an inherent property of the oligomerization domain peptide. It was noted that the full-length X-protein caused a higher level of apoptosis (20-30% increase in cell apoptosis) than the truncated X-protein.

### The Oligomerization Domain Peptide (aa 16-35) Disrupts the Tertiary Structure of the X-Protein

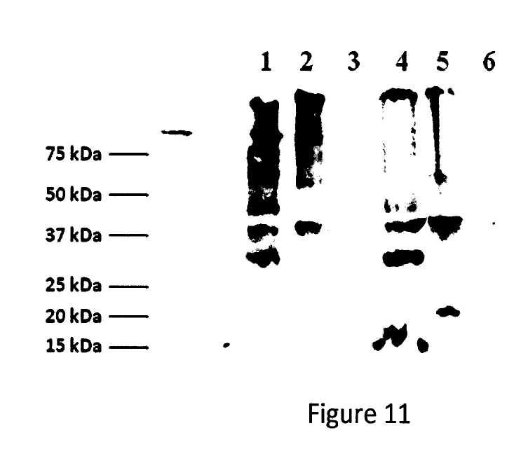

The inventors investigated how the oligomerization domain peptide (aa 16-35) antagonized X-protein-mediated function. The aim was to determine whether the peptide disrupted the tertiary structure of the X-protein. HepG2 cells were transfected with either the pCDNA3.1-HBX, pCDNA3-HBX Myc or pCDNA3-GFP plasmids and were treated thrice over 48 h with the oligomerization domain peptide (aa 16-35). Control transfectants were not treated with peptide. The nuclear subfraction was resolved by SDS-PAGE using non-reducing loading buffer to keep the disulphide bonding of the X-protein intact. The proteins were transferred onto a PVDF membrane, and probed with a mouse antihuman X-protein primary antibody, followed by a secondary goat anti-mouse HRP-conjugated antibody. The oligomerization domain peptide (aa 16-35) did not decrease the overall level of the X-protein, ruling out a decrease in X-protein expression as a mechanism for disruption of apoptosis (FIG. 11). Transfectants not treated with peptide expressed higher order forms of the X-protein, including the dimeric form at ˜34 kDa, and apparently even higher order structures (FIG. 11). In contrast, cells transfected with pCDNA3.1-HBX and pCDNA3-HBX Myc and treated with the oligomerization domain peptide expressed monomeric forms at ˜17 kDa and ˜21 kDa, respectively. They also expressed dimeric forms of the X-protein at ˜34 kDa, but did not express the higher order forms. The results indicate that the oligomerization domain peptide disrupts the tertiary structure of the X-protein, preventing dimerization and the formation of higher order structures. The X-protein is postulated to function as a dimer, hence disruption of its dimerization may be the mechanism by which the oligomerization domain peptide (aa 16-35) antagonizes the proapoptotic function of the X-protein.

### Cell Permeability of Oligomerization Domain Peptides

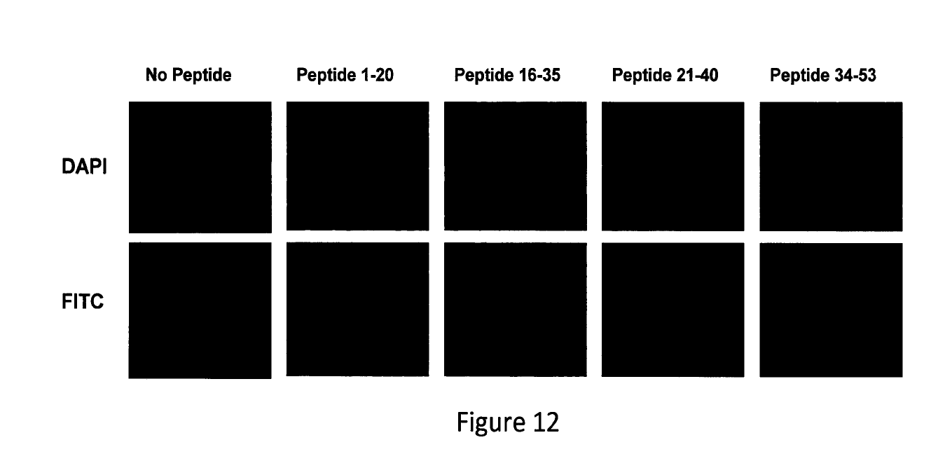

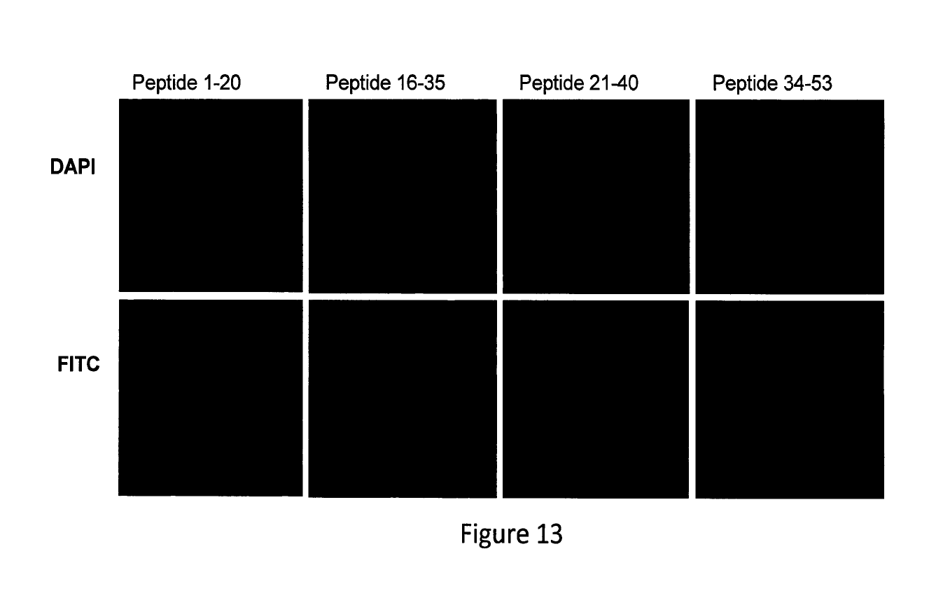

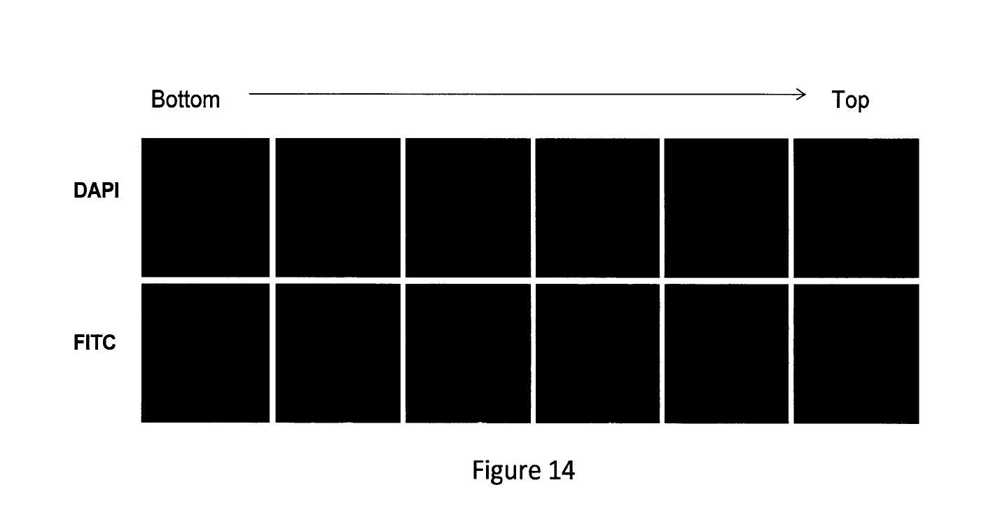

The oligomerization domain peptide aa 16-35 was serendipitously discovered to be able to spontaneously enter HepG2 cells without fusion to an octa-arginine carrier peptide. This suggests that the oligomerization domain of the X-protein contains a protein transduction domain as found in the TAT protein expressed by HIV. Four FITC-labelled peptides encompassing aa 1-20, 16-35, 21-40 and 34-53 from the N-terminal region of the X-protein were incubated with HepG2 cells and their uptake by the cells recorded using a Nikon E600 fluorescence microscope. Peptides aa 1-20 and aa 16-35 were the only peptides of the four peptides tested that were readily taken up by the cells (FIG. 12). Low fluorescence by cells treated with the remaining two peptides could possibly be attributed to non-specific binding of the peptides to the cell-surface (FIG. 12). Control cells which were not treated with peptide did not fluoresce. The above cells were examined by confocal microscopy to gain a better idea of the distribution of the peptides. Cross-sectional analysis of the cells confirmed that peptides aa 1-20 and aa 16-35 were taken up into the cell cytoplasm, as well as into the nucleus (FIGS. 13 and 14). Peptides aa 21-40 and 34-53 were not cell-permeable (FIG. 13).

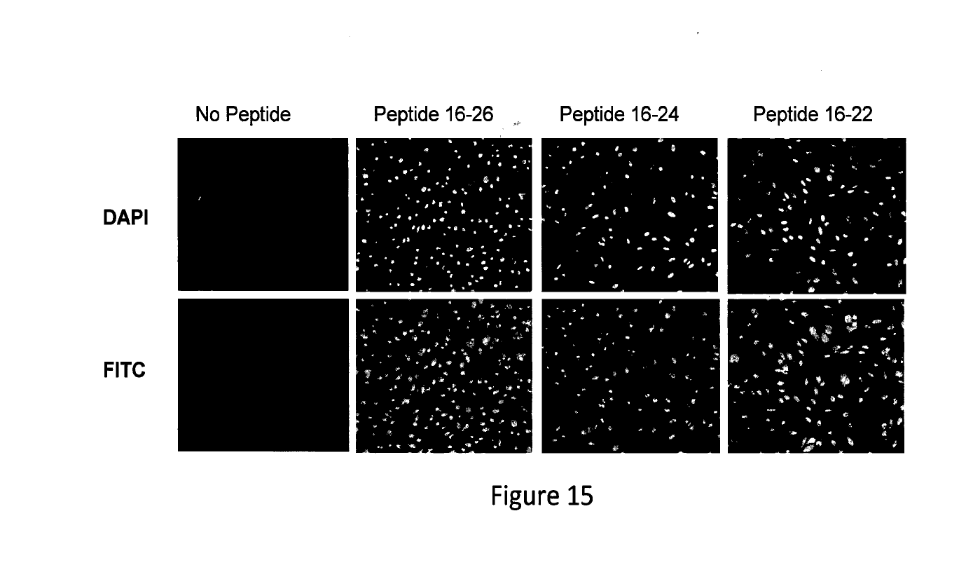

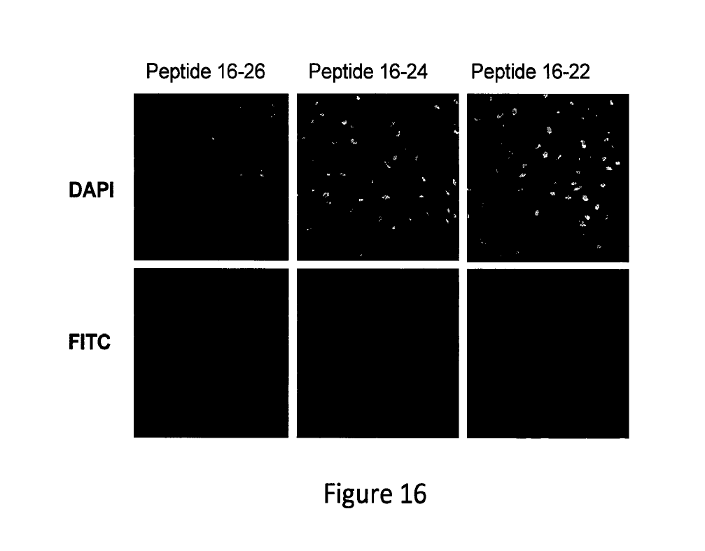

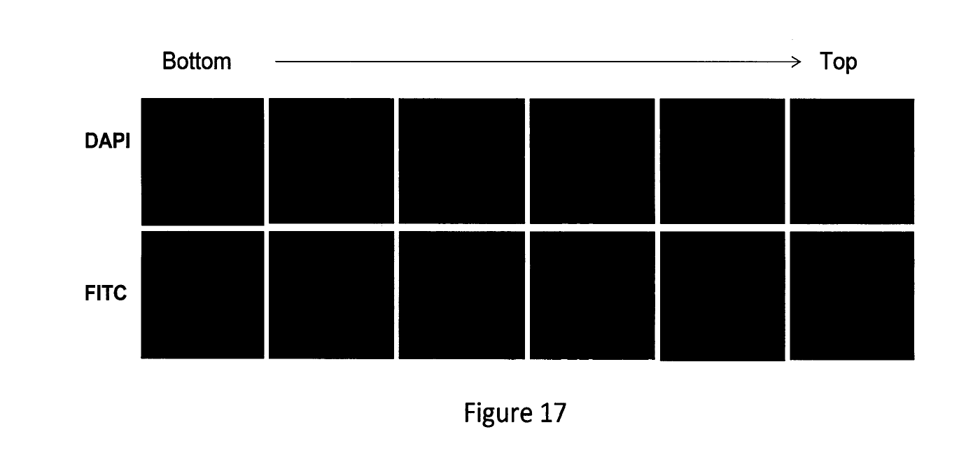

Peptide aa 16-35 was divided into the smaller fluoresceinated peptides aa 16-26, 16-24 and 16-22 which were tested for their ability to enter HepG2 cells. All three peptides were cell-permeable, being taken up by cells at a level equivalent to that of the parental peptide aa 16-35 (FIG. 15). Confocal microscopy confirmed that all three peptides were taken up by cells (FIG. 16). Confocal slices taken through the cells established that peptide aa 16-22 was taken up into both the cytoplasm and nucleus of cells (FIG. 17).

Entry of the Cell Permeable Peptide into HepG2 Cells is Dependent on Heparin Binding

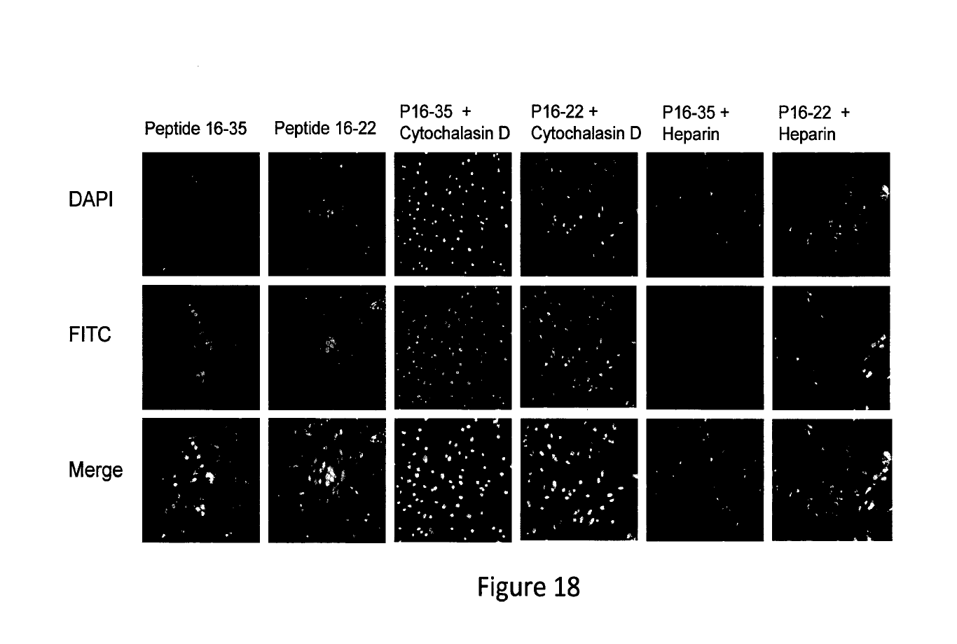

Exactly how cell-permeable peptides pass through the plasma membrane of cells is still not fully understood. It has been suggested that some peptides gain entry via macropinocytosis, which is an endocytic process driven by actin. Actin elongation on the cell surface leads to an extension of the plasma membrane into the extracellular environment. When the plasma membrane fuses back with itself it forms macropinosomes, and in doing so encapsulates and internalizes a large volume of extracellular fluid. Another mechanism is via the binding of peptides to cell surface heparan sulphate proteoglycans, which leads to internalization of the peptide through an endocytic process. HepG2 cells were pretreated with cytochalasin D and heparin, and then incubated with FITC-labelled cell permeable peptides aa 16-22, and aa 16-35 for 3 h to determine whether either of the above two mechanisms mediated cell uptake of the X-protein peptides. Control cells were treated with the X-protein peptides but were not treated with either chemical. Cytochalasin D is an F-actin elongation inhibitor, which inhibits macropinocytosis by inhibiting extension of the plasma membrane. Heparin would be expected to antagonize the binding of the peptides to cell surface heparan sulphate proteoglycans. As shown in FIG. 18 pretreatment of cells with cytochalasin D had no effect on the entry of the peptides into the cell, indicating macropinocytosis is not involved in peptide internalization. In contrast, pretreatment with heparin effectively blocked peptide entry into the cells, indicating that heparin binding of peptides to the cell surface plays an important role in cell entry by the X-protein peptides (FIG. 18).

### Short N-Terminal Peptides aa 1-8, 1-15 and 16-20 are Cell-Permeable.

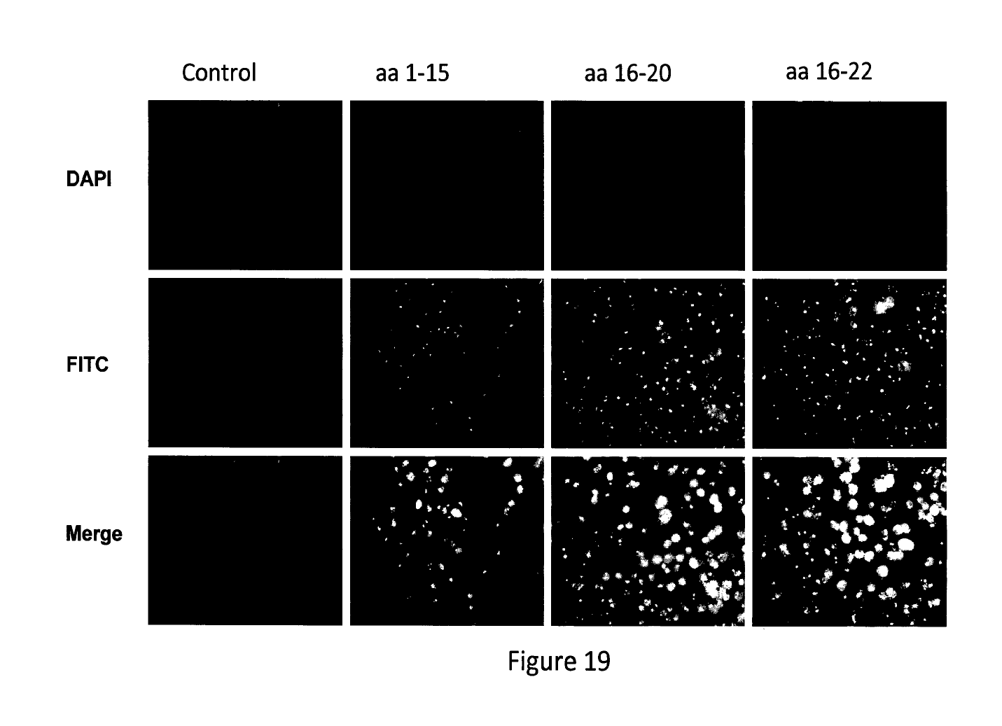

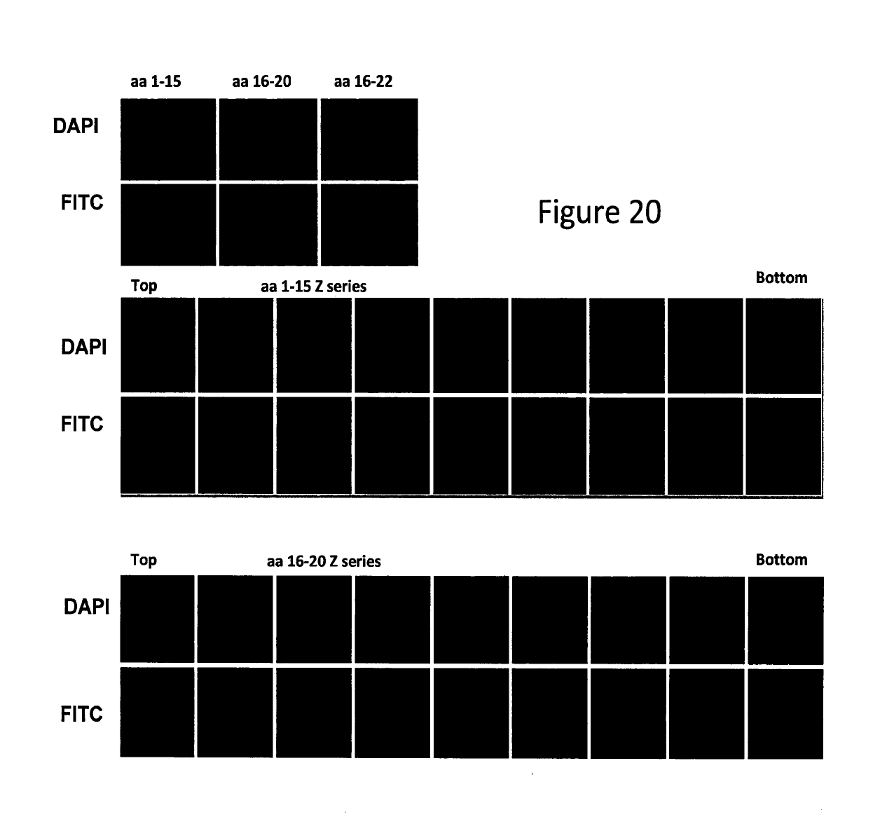

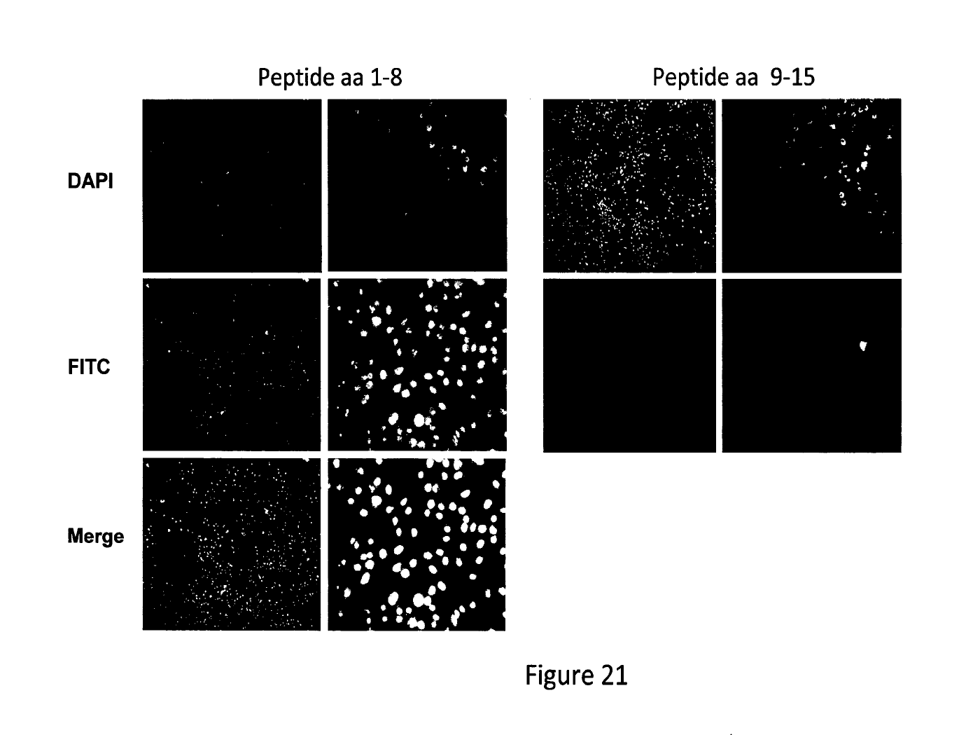

Fluoresceinated peptides aa 1-15, 16-20, and 16-22 were incubated with HepG2 cells and their uptake by the cells recorded using a Nikon E600 fluorescence microscope. All three peptides were taken up into the cells (FIG. 19). There appeared to be no difference in the efficiency of uptake of peptide aa 16-20 versus peptide aa 16-22. Confocal microscopy confirms that peptides aa 1-15 and 16-20 are cell-permeable. Peptide uptake was confirmed using a Leica TCS-SP2 confocal microscope (FIG. 20, upper panel). Confocal slicing of the cells revealed that peptides aa 1-15 and 16-22 were taken up into the cytoplasm and nucleus (FIG. 20, lower 2 panels). The N-terminal peptide aa 1-15 was divided into two, and each fluoresceinated peptide tested for uptake into HepG2 cells. Only peptide aa 1-8 proved to be cell-permeable (FIG. 21).

### The X-Protein Cell-Permeable Peptide 16-22 is Able to Enter Several Different Adherent Cell Lines, but not the Nonadherent Cell Line TK1

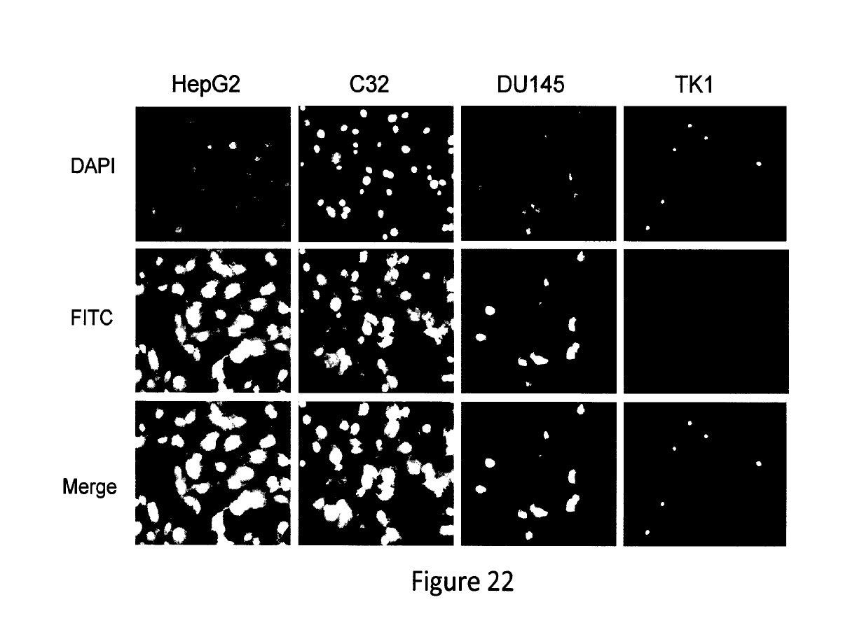

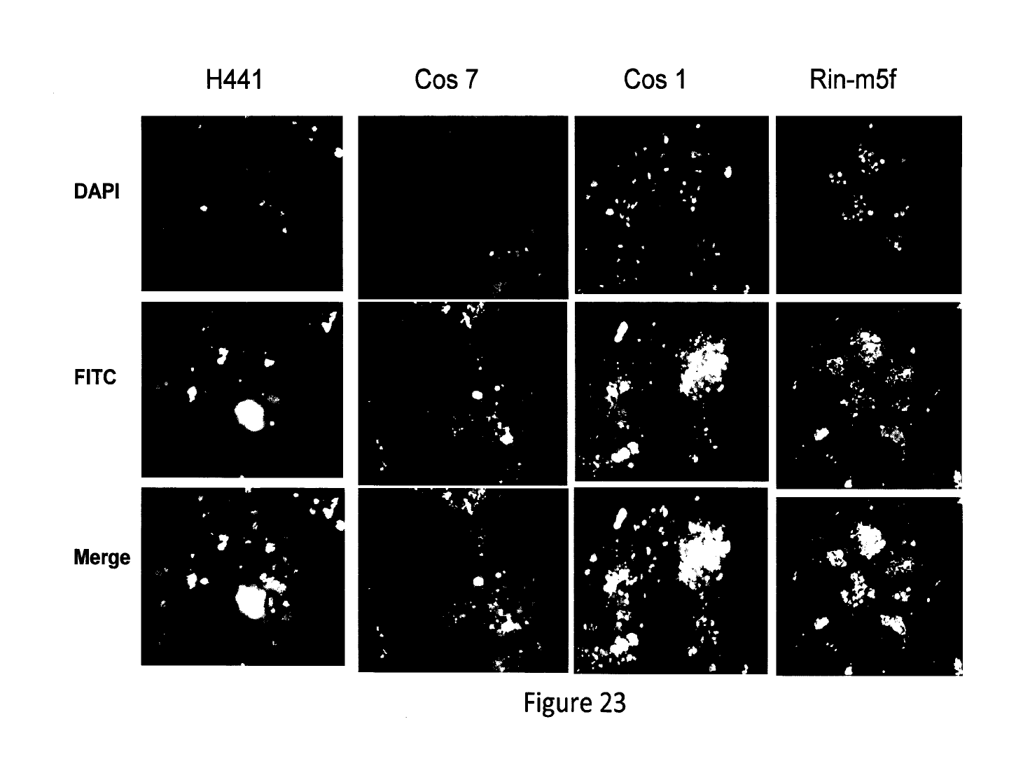

The X-protein cell-permeable peptide aa 16-22 was tested for its ability to be taken up by other mammalian cell lines including the human C32 melanoma, DU145 prostate carcinoma, TK1 T cell lymphoma, H441 lung carcinoma, Rin-m5f rat pancreatic islet tumor and Cos-1/7 green monkey kidney cell lines. TK1 cells are suspension cells whereas all other cell lines are adherent cells. Cells were incubated with the fluoresceinated X-protein cell-permeable peptide 16-22 for 3 h in medium appropriate for that cell line in the absence of FCS. The HepG2 cell line was used as a positive control. The X-protein peptide was able enter all adherent cell lines regardless of type or source of the cell (FIGS. 22 and 23). The HepG2, C32, and DU145 cell lines showed efficient (>90% cells positive) uptake of the peptide confirming the peptide was cell permeable (FIG. 22). In contrast, the peptide was taken up less efficiently (60 to 70% cells positive) into the H441, Cos-7, Cos-1 and Rin-m5f cell lines (FIG. 23). The peptide was completely unable to enter the suspension cell line TK1 (FIG. 22).

### The X-Protein Cell-Permeable Peptide 16-22 Cannot Enter Unattached Monocytes and Lymphocytes

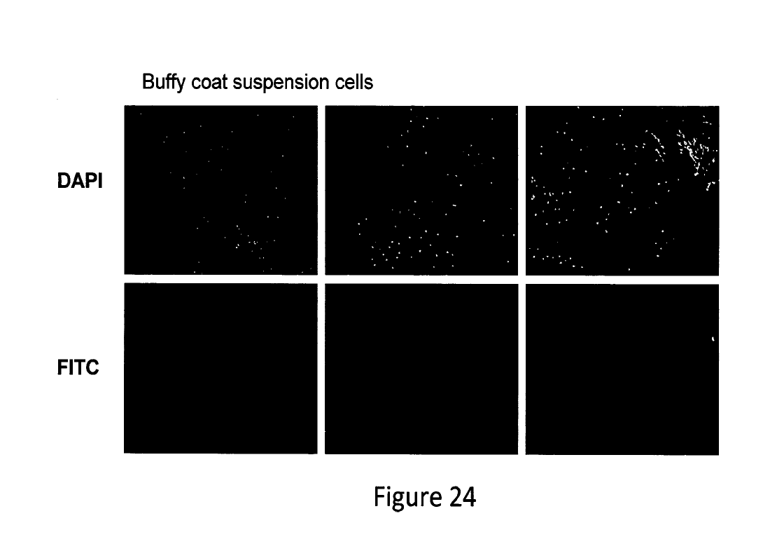

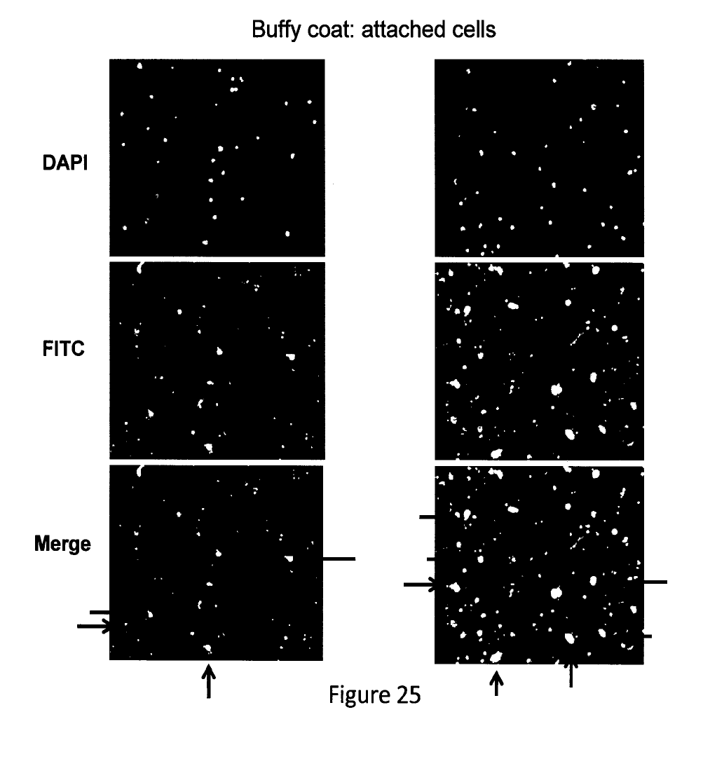

The previous experiment indicated that peptide aa 16-22 is not able to enter the suspension cell line TK1. To determine whether this was a general feature of nonadherent cells, the ability of the peptide to enter non-adherent human peripheral blood mononuclear cells was examined. A buffy coat containing monocytes, lymphocytes, and platelets was isolated from 10 ml of human blood and incubated in 8-chamber slides for 3 h with the cell-permeable peptide aa 16-22 in RPMI medium without FCS. The nonadherent buffy coat cells did not fluoresce indicating that they did not take up the peptide (FIG. 24). In contrast, monocytes and platelets that had adhered to the bottom of the 8-chamber slides fluoresced indicating they had taken up the peptide (FIG. 25).

The X-Protein Cell-Permeable Peptide aa 16-22 does not Enter Nonadherent Red Blood Cells and Platelets

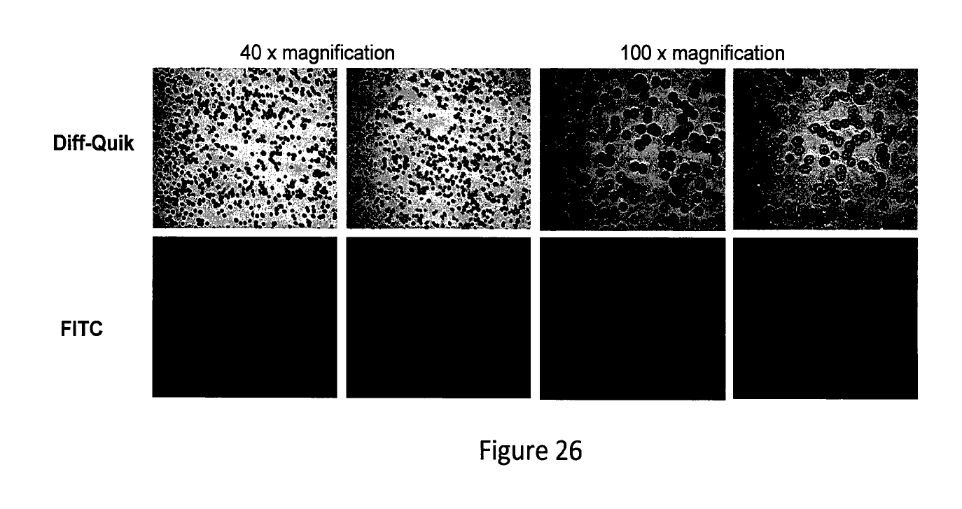

Whole blood was incubated with the fluoresceinated X-protein cell-permeable peptide aa 16-22 for 3 h to determine whether erythrocytes and platelets are able to take up the peptide. Blood was collected from a finger prick and suspended in 10 mM citrate buffer to prevent the blood clotting. After incubation with peptide the cells were washed in PBS and a blood smear prepared on a glass slide. The cells were stained with Diff-Quik. None of the cell types present, the majority of which were erythrocytes (majority of large cells had doughnut appearance) and platelets were able to take up the cell-permeable peptide (FIG. 26).

### The X-Protein Cell-Permeable Peptide aa 16-22 is Able to Enter Attached TK1 Cells

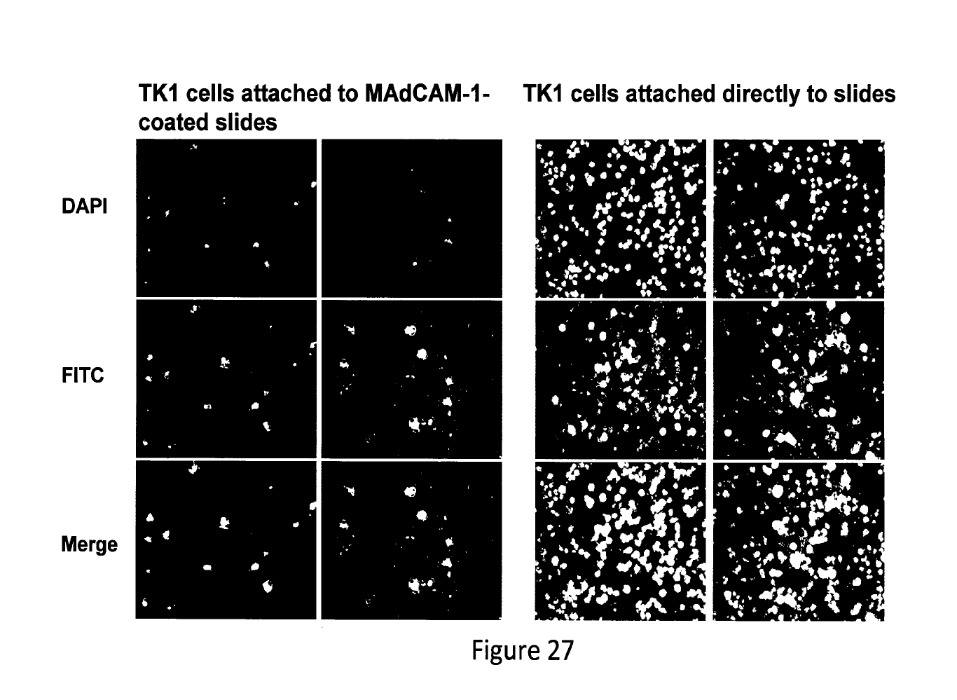

As above, the lymphocytic cell line TK1 in suspension was not able to take up the X-protein cell-permeable peptide aa 16-22. Once activated, circulating lymphocytes have the ability to adhere to the vascular endothelium, to attach to antigen presenting cells, and to attach to and move through the extracellular matrix. The fact that adherent monocytes took up the peptide raised the possibility that TK1 T cells attached to a surface might also be able to take up the X-protein cell permeable peptide aa 16-22. TK1 cells were adhered to MAdCAM-1-coated glass slides or left to bind directly to the surface of the glass slides. Cells attached to the slides were incubated with the X-protein cell-permeable peptide aa 16-22 for 3 h in RPMI without FCS. Virtually all TK1 cells attached via α4β7 to MAdCAM-1 took up the peptide, whereas only 50 to 60% of cells directly attached to the glass slide took up the peptide (FIG. 27).

X-Protein Cell-Permeable Peptide aa 16-22 is Able to Carry a Foreign Peptide into Adherent TK-1 T Cells

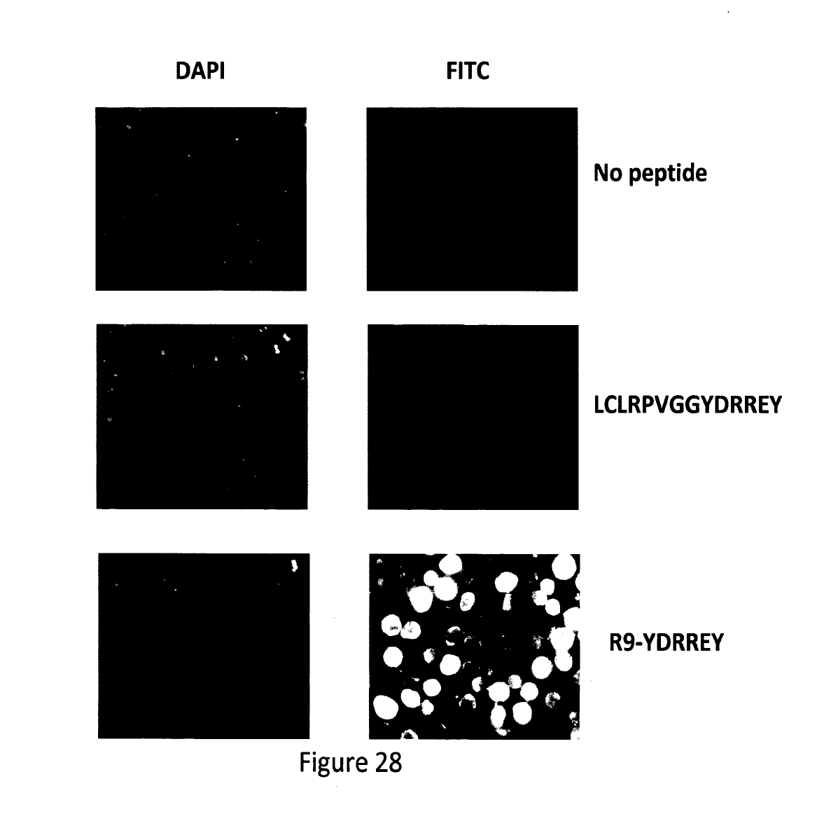

The X-protein cell-permeable peptide LCLRPVG (SEQ ID NO: 2) was fused to the hexapeptide YDRREY (SEQ ID NO 16) taken from the cytoplasmic domain of the human integrin β7 subunit. The peptides were separated by a glycine linker, and a biotin tag was added N-terminally. The fusion peptide was compared with the YDRREY (SEQ ID NO 16) peptide fused to an R9 (SEQ ID NO: 145) carrier peptide for its ability to enter TK-1 T cells adherent to MAdCAM-1. Both peptides were taken up into TK-1 cells (FIG. 28). Whilst the R9YDRREY (SEQ ID NO 14) peptide entered all the cells, it appeared to be preferentially taken up in greater amounts by a specific subpopulation of cells. In contrast, the LCLRPVGGYDRREY (SEQ ID NO 15) peptide was uniformly taken up in similar amounts by all cells.

### Example 2

### Materials and Methods

### Materials

The peptides biotin-LCLRPVGGGRRRQQQQQQRRR (SEQ ID NO 17), biotin-RRRRRRRRMAARLCCQLDPARDVLCLRP (SEQ ID NO 20), and biotin-RRRRRRRRHKLVRSPAPCKFFTSAGGLNVTAPTPQQLQAFKNEVGVLRK (SEQ ID NO 19) were synthesized by Peptide 2.0. The oligonucleotide 5AmMC12-GAGCTGCACGCTGCCGTC-Tex615 (SEQ ID NO 18) was synthesized by Integrated DNA Technologies. FITC-conjugated rabbit IgG was purchased from Sigma (cat# F9887). The C32 melanoma cell line (Cat# CRL-1585), Wm 266-4 melanoma cell line (Cat# CRL-1676), and HepG2 liver cancer cell line (Cat# HB-8065) were purchased from the American Type Culture Collection.

### Methods

### Purification of Transglutaminase

Activa dairy powder (ACTIVA TG-BP-MH, Kerry Ingredients, Otahuhu, Auckland; purchased from Lesnie's, Tamaki, Auckland) which contains a mixture of casein and transglutaminase was dissolved in 10 ml of H₂O to a concentration of 20 mg/ml. Two millilitres of 20 mM sodium acetate buffer (pH 4.0) was added with mixing, and the solution was centrifuged at 13,000 g for 30 min. The supernatant containing only transglutaminase was dialyzed against PBS (pH 7.4), and the enzyme was concentrated using Aquacide. Analysis of the isolated enzyme by electrophoresis on a 12% polyacrylamide SDS gel revealed a single band of ˜37 kDa, as expected.³²

### Cross-Linking of Cargoes to the X-Peptide Using Transglutaminase

Reaction buffer (30 μl) consisting of 5 mM CaCl₂, 1 mM DTT and 50 mM Tris-HCl was mixed with 1.5 μl of 200 μM X-protein cell-permeable peptide, and either 3 μl of 100 μM rabbit IgG-FITC or 3 μl of 100 μM 18mer GFP antisense oligo to give final concentrations of the cargoes of 8.7 Transglutaminase was then added at a concentration of 100 μg/ml. The solutions were incubated at 37° C. for 1 h, and then the reaction was stopped by addition of EDTA to 100 mM.

Testing the Ability of the X-Protein Carrier Peptide to Deliver Rabbit IgG-FITC and a 18mer GFP Antisense Oligo into HepG2 Cells

HepG2 cells were seeded at 1×10⁵ cells per well into 8-well chamber slides overnight at 37° C. and 5% CO₂. The next day the cells were washed thrice with serum-free MEM, resuspended in 500 μl of the same medium, and added to the wells. The X-protein carrier peptide cross-linked to rabbit IgG-FITC and to the GFP antisense oligo were added to the cells at final concentrations of 0.5 μM, and incubated at 37° for 3 h. In an additional experiment, the X-protein carrier peptide conjugated to the 18mer oligo was incubated with cells for 24 h, 3 h, and 30 min in order to determine the timing of uptake of the conjugate into cells. The unconjugated oligo was added to cells for 3 h as a control. The cells were washed thrice with PBS, and fixed with 4% formaldehyde. They were washed thrice with PBS, DAPI added for visualization of cell nuclei, followed by examination by fluorescence microscopy for entry of the fluorescent cargoes.

Testing Different Ratios of Conjugation of the X-Protein Carrier Peptide and 18mer GFP Antisense Oligo for Entry into HepG2 Cells

HepG2 cells were seeded at 1×10⁵ cells per well into 8-well chamber slides overnight at 37° C. and 5% CO₂. The next day the cells were washed thrice with serum-free MEM, resuspended in 500 μl of the same medium. Aliquots of 0.75, 1.5, and 3 μl of a 100 μM solution of the 18mer oligo were mixed with 1.5 μl of 200 μM of X-protein carrier (final concentration of 8.7 μM) peptide to give final concentrations of 2.3, 4.5, and 8.7 μM of oligo, followed by cross-linking with transglutaminase. The X-protein carrier peptide conjugated to the 18mer oligo was added to the cells at final concentrations of the oligo of 0.14, 0.3, 0.6 μM, and incubated at 37° for 3 h. The cells were fixed and stained with DAPI for fluorescence microscopy, as above.

### Testing the Ability of a B-Raf Targeting Peptide to Kill Melanoma Cells

The melanoma cell lines WM-266-4 and C32 were seeded into 8-well chamber slides at 1×10⁵ cells per well in full MEM media and incubated at 37° C. and 5% CO₂ overnight. The next day the cells were washed thrice with serum-free MEM, resuspended in 500 μl of the same medium, and added to the wells. The B-Raf X-protein fusion peptide (biotin-RRRRRRRRHKLVRSPAPCKFFTSAGGLNVTAPTPQQLQAFKNEVGVLRK (SEQ ID NO 19)) and a control cell-permeable peptide (biotin-RRRRRRRRMAARLCCQLDPARDVLCLRP (SEQ ID NO 20)) which does not cause apoptosis were added to the C32 and WM-266-4 melanoma cells to final concentrations of 10 μM and 20 μM, respectively, followed by incubation for 3 h at 37° C. Annexin-V labelling solution was prepared by adding 20 μl of Annexin V labelling reagent and 10 μl of propidium iodide in 1 ml of incubation buffer. The cells were washed with incubation buffer, and 100 μl of Annexin-V labelling solution was added, followed by incubation for 15 min in the dark. The cells were washed with incubation buffer, fixed with 4% formaldehyde in PBS for 30 min, washed with PBS and slide chambers removed. A drop of Prolong Gold anti-fade reagent with DAPI was added to each sample, the slides dried overnight, and then examined by fluorescence microscopy.

### Results

Refining the Size of the Oligomerization Domain of the X-Protein (to be Placed after the Relevant Section in the Provisional)

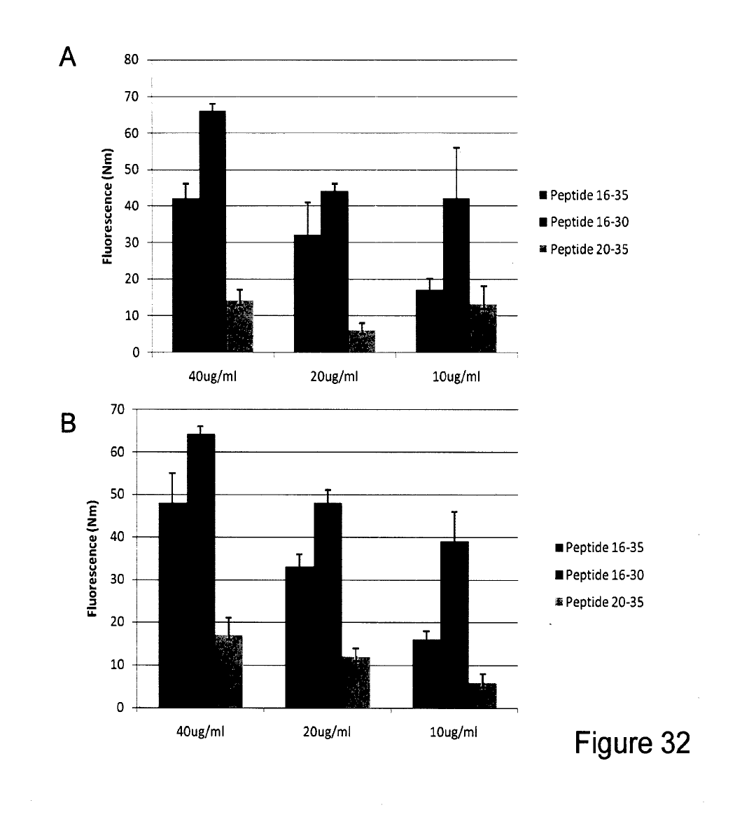

Two smaller peptides encompassing region 16-35 were synthesized to determine whether the aa 16-35 was the minimum peptide motif needed for X-protein dimerization. These peptides were truncated N- and C-terminally, namely aa 20-35 and 16-30. They were analyzed using the same peptide binding assay. Peptide 16-30 bound peptide 1-50 even more strongly than peptide 16-35, whereas peptide 20-35 showed only weak binding (FIG. 32). Thus of the peptides examined, the minimal peptide motif needed for dimerization of the X-protein is the 15 aa peptide 16-30 (SEQ ID NO 90).

X-Protein Cell-Permeable Peptide aa 16-22 is Able to Carry a Conjugated Rabbit IgG into HepG2 Cells

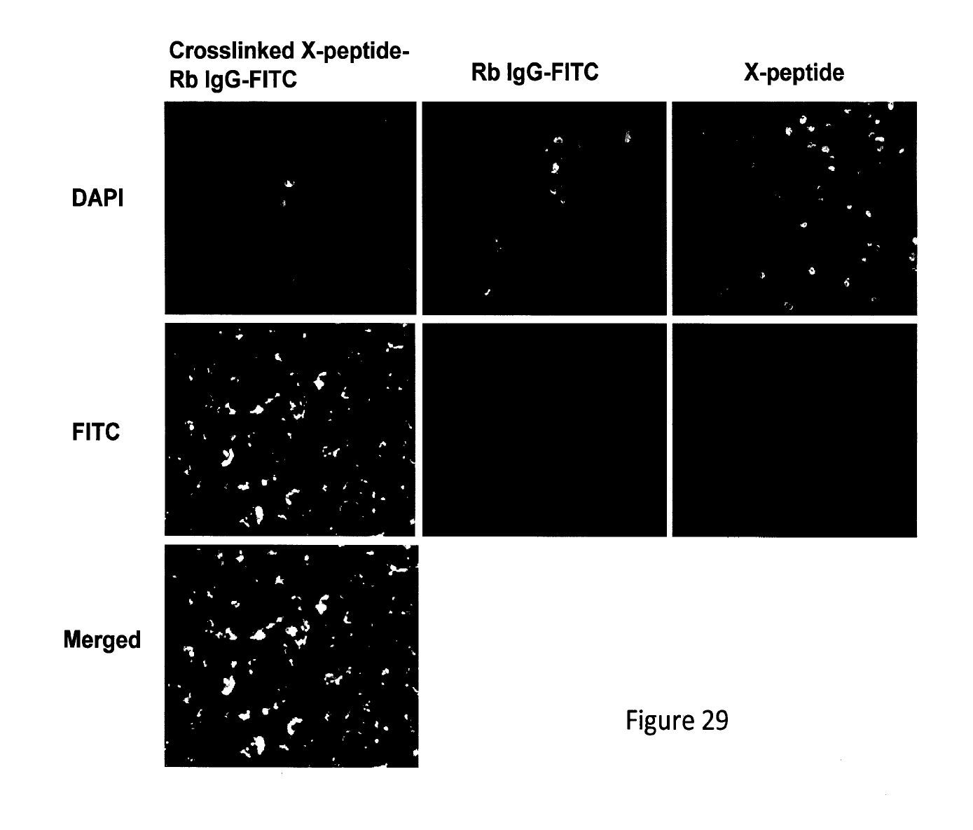

The X-protein cell-permeable peptide LCLRPVG (SEQ ID NO 2) fused to a polyglutamine stretch (biotin-LCLRPVGGGRRRQQQQQQRRR (SEQ ID NO 17)) was conjugated to FITC-labelled rabbit IgG using transglutaminase. The peptides were separated by a glycine linker, the polyglutamine stretch was surrounded by arginine residues to aid solubility, and a biotin tag was added N-terminally. The X-protein cell-permeable peptide conjugated to rabbit IgG was not purified, but simply added to HepG2 cells. It was readily taken up by HepG2 cells, whereas unconjugated FITC-labelled rabbit IgG was not (FIG. 29).

X-Protein Cell-Permeable Peptide aa 16-22 is Able to Carry an 18mer Oligonucleotide into HepG2 Cells

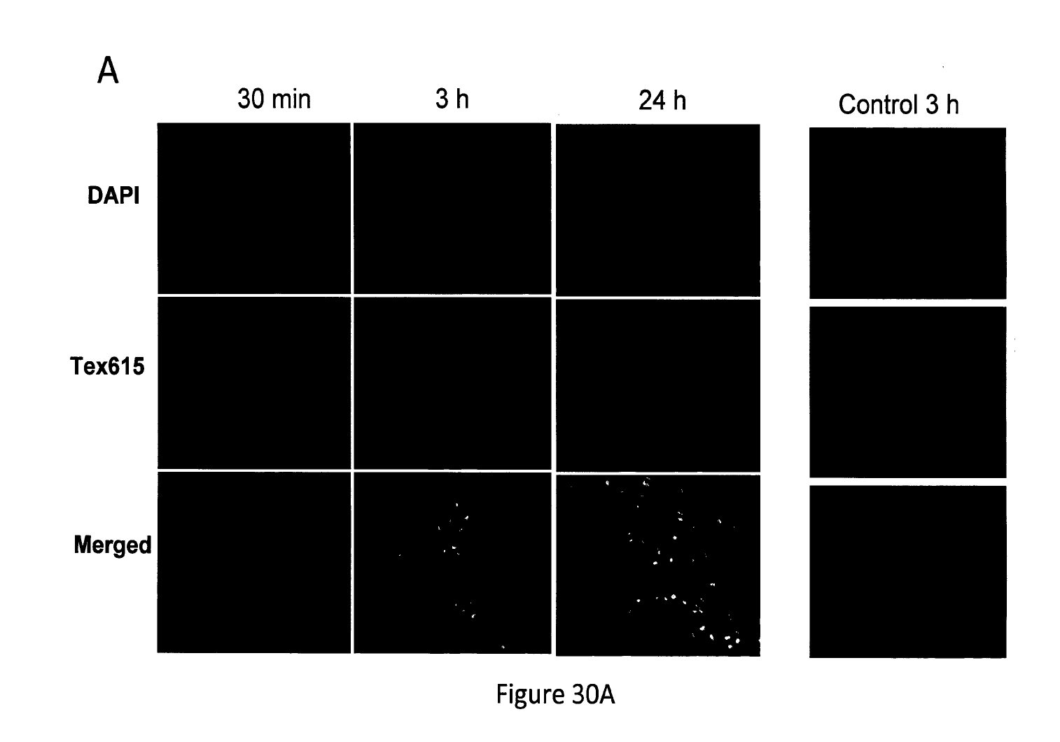

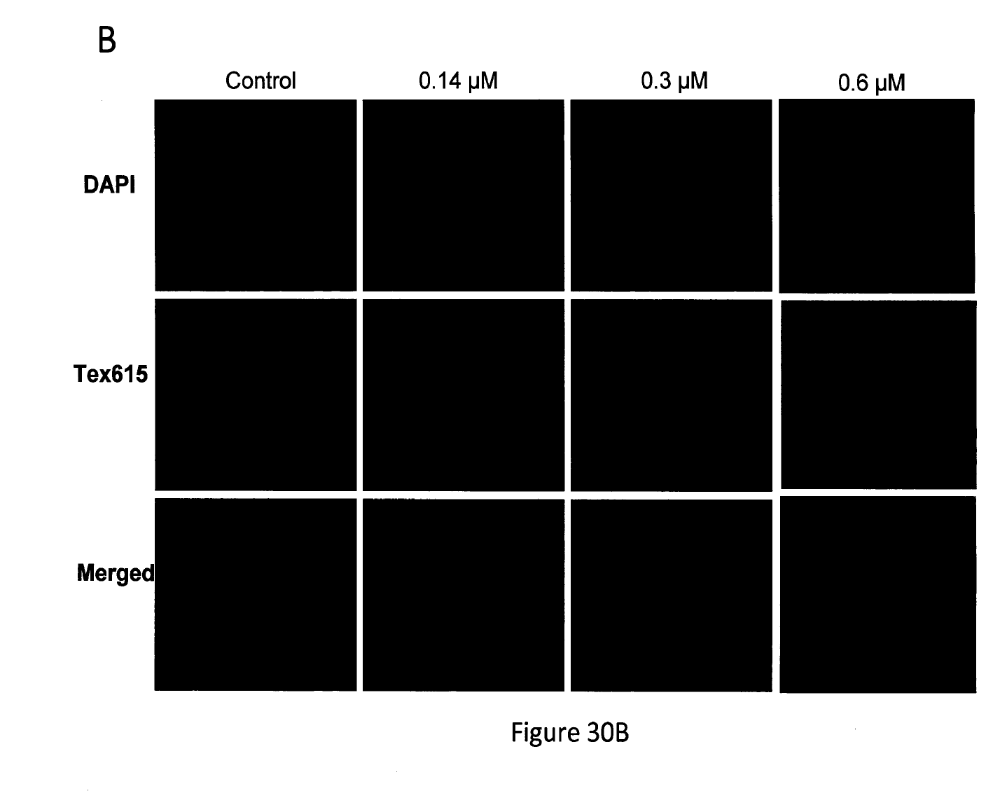

The X-protein cell-permeable peptide LCLRPVG (SEQ ID NO 2) fused to a polyglutamine stretch (biotin-LCLRPVGGGRRRQQQQQQRRR (SEQ ID NO 17)) was conjugated to the Tex615-labelled 18mer oligonucleotide 5AmMC12-GAGCTGCACGCTGCCGTC (SEQ ID NO 18) containing a 5′-amino group using transglutaminase. The X-protein cell-permeable peptide conjugated to the 18mer oligonucleotide was not purified, but simply added to HepG2 cells at a final concentration of 8.7 μM. It was readily taken up by HepG2 cells within 30 min, whereas the unconjugated Tex615-labelled 18mer oligonucleotide at 8.7 μM was not (FIG. 30A). The 18 mer oligo was conjugated to the carrier peptide at differing ratios, with the optimal ratio for cell uptake resulting from conjugation of 4.5 to 8.7 μM oligo to 10 μM carrier peptide, giving final concentrations in solution of 0.3 and 0.6 μM (FIG. 30B).

A Cell-Permeable Carrier Peptide is Able to Carry an Anti-Cancer Peptide into Cancer Cells Leading to Cell Death

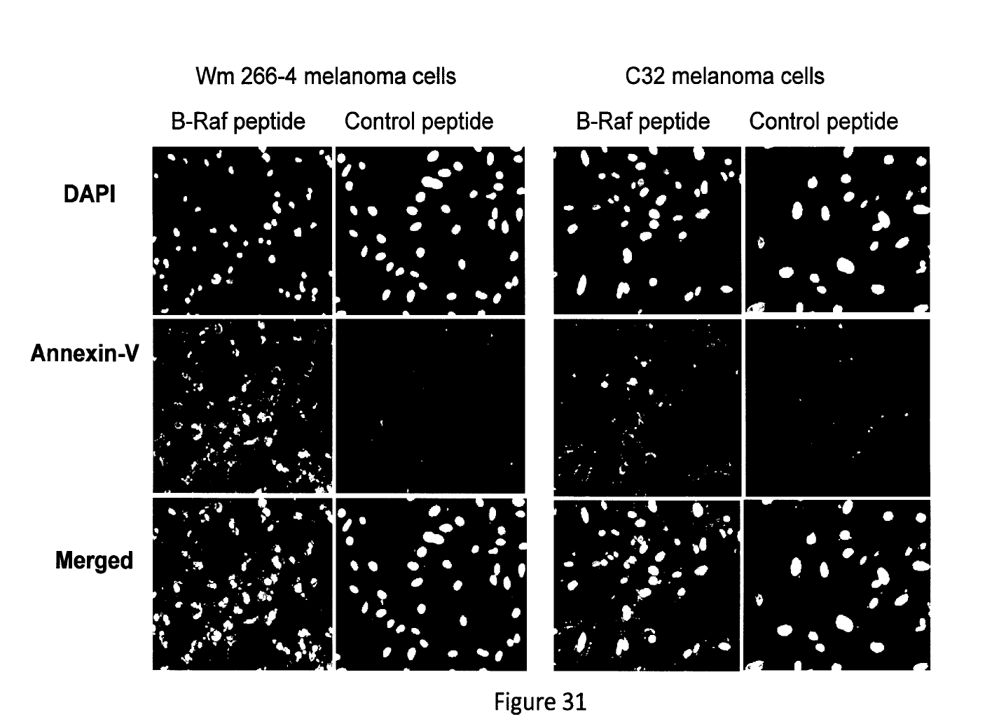

The growth of ˜50% of all melanomas is driven by a mutation (V600→E) in the B-Raf gene, and 25% by overexpression of wild-type B-Raf.²⁷⁻²⁹ PLX4032, a small-molecule inhibitor specific for mutated B-Raf recently raised excitement as it caused unprecedented regression of melanomas in 80% of patients.³⁰ Hence, approaches to target the B-Raf oncoprotein hold promise in the treatment of melanoma. Here we have developed a peptide capable of binding to the dimerization interface of B-Raf, and causing destruction of B-Raf. The polyarg (R8: SEQ ID NO: 144) cell-permeable peptide was fused to the degradation domain of the X-protein and to a dimerization domain (LNVTAPTPQQLQAFKNEVGVLRK (SEQ ID NO 89)) from the B-Raf protein.³¹ A biotin tag was included at the N-terminus, giving the peptide biotin-RRRRRRRRHKLVRSPAPCKFFTSAGGLNVTAPTPQQLQAFKNEVGVLRK (SEQ ID NO 19). It was expected that the peptide would be taken up by melanoma cells, bind to B-Raf targeting it for polyubiquitin-mediated destruction, leading to death of the cells. Indeed, addition of the peptide to WM-266-4 and C32 melanoma cells led to tumour cell death, whereas the control peptide biotin-RRRRRRRRMAARLCCQLDPARDVLCLRP (SEQ ID NO 20) had no affect (FIG. 31).

A Cell-Permeable Carrier Peptide is Able to Carry an Anti-B-Raf Antibody into Melanoma Cells Leading to Cell Death

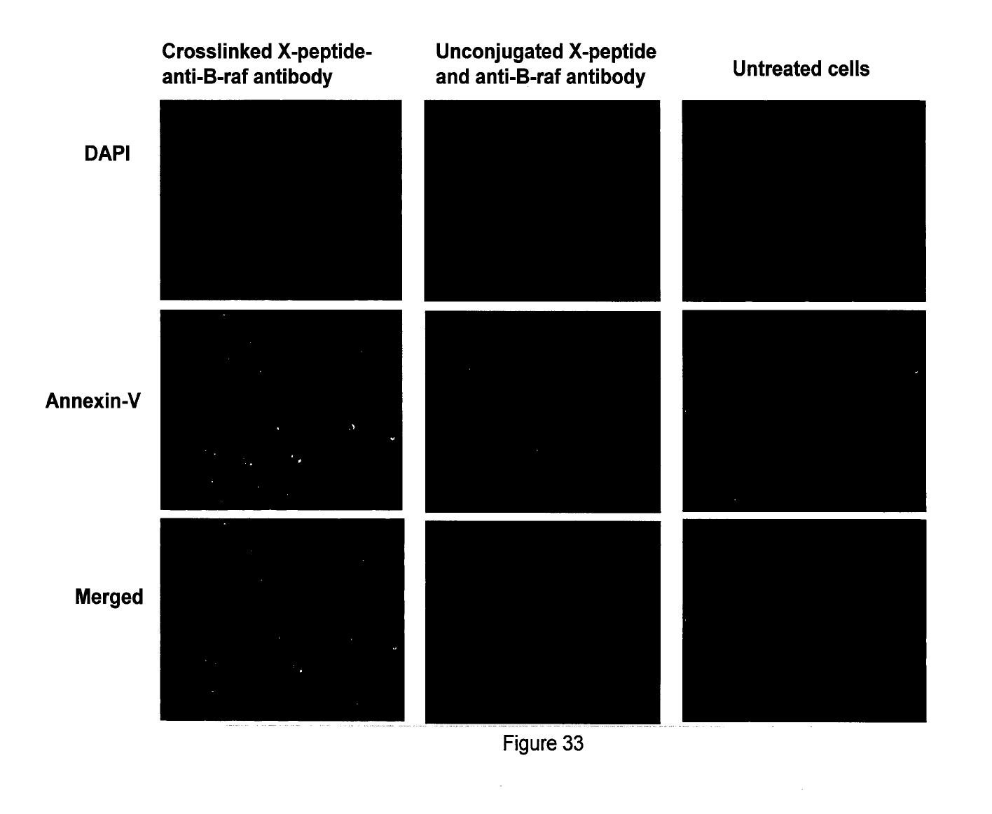

Here we delivered a rabbit polyclonal anti-human B-raf antibody to WM-266-4 melanoma cells using the X-protein aa 16-22 carrier peptide LCLRPVG (SEQ ID NO: 2), given the previous finding that the carrier peptide can intracellularly deliver an antibody to cells. The anti-B-raf antibody delivered by the X-protein carrier peptide caused the cells to undergo apoptosis as detected by Annexin-V fluos staining, whereas in contrast unconjugated B-raf antibody and carrier peptide had no detectable effect (FIG. 33).

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features. Furthermore, titles, headings, or the like are provided to enhance the reader's comprehension of this document, and should not be read as limiting the scope of the present invention. The entire disclosures of all applications, patents and publications, cited above and below, if any, are hereby incorporated by reference. However, the reference to any prior art in this specification is not, and should not be taken as, an acknowledgment or any form of suggestion that that prior art forms part of the common general knowledge in any country in the world. Throughout this specification (and any claims which follow), unless the context requires otherwise, the words “comprise”, “comprising” and the like, are to be construed in an inclusive sense as opposed to an exclusive sense, that is to say, in the sense of “including, but not limited to”.

### REFERENCES

1. Kumar V, Jayasuryan N, Kumar R. A truncated mutant (residues 58-140) of the hepatitis B virus X protein retains transactivation function. Proc Natl Acad Sci USA 93:5647-52, 1996.
2. Misra K P, Mukherji A, Kumar V. The conserved amino-terminal region (amino acids 1-20) of the hepatitis B virus X protein shows a transrepression function. Virus Res 105:157-65, 2004.
3. Zanetti A R, Van Damme P, Shouval D. The global impact of vaccination against hepatitis B: a historical overview. Vaccine 26:6266-73, 2008.
4. Lodato F, Mazzella G, Festi D, et al: Hepatocellular carcinoma prevention: A worldwide emergence between the opulence of developed countries and the economic constraints of developing nations. World J Gastroenterol 12:7239-7249, 2006.
5. Murakami, S. Hepatitis B virus X-protein: Structure, function, and biology. Intervirol 42: 81-99, 1999.
6. Lee A T, Lee C G. Oncogenesis and transforming viruses: the hepatitis B virus and hepatocellularcarcinoma—the etiopathogenic link. Front Biosci 12: 234-45, 2007.
7. Newell P, Villanueva A, Llovet J M. Molecular targeted therapies in hepatocellular carcinoma: From pre-clinical models to clinical trials. J Hepatol 49: 1-5, 2008.
8. Pang R W, Poon R T. From Molecular biology to targeted therapies for hepatocellular carcinoma: The future is now. Oncology 72 Suppl 1: 30-44, 2007.
9. Koike, K., Moriya, K., Iino, S., Yotsuyanagi, H., Endo, Y., Miyamura, T., and Kurokawa, K. High-level expression of hepatitis B virus HBX-gene and hepatocarcinogenesis in transgenic mice. Hepatol 19: 810-819, 1994.
10. Madden, C. R., Finegold, M. J., and Slagle, B. L. Hepatitis B virus X-protein acts as a tumour promoter in development of diethylnitrosamine-induced preneoplastic lesions. J Virol 75: 3851-3858, 2001.
11. Zhu H, Wang Y, Chen J, Cheng G, Xue J. Transgenic mice expressing hepatitis B virus X protein are more susceptible to carcinogen induced hepatocarcinogenesis. Exp Mol Pathol 76:44-50, 2004.
12. Luber, B., Arnold, N., Sturzl, M., Hohne, M., Schirmacher, P., Lauer, U., Wienberg, J., Hofschneider, P., and Kekule, A. Hepatoma derived integrated HBV DNA causes multi-stage transformation in vitro. Oncogene 12: 1597-1608, 1996.
13. Feitelson, M. A. and Clayton, M. M. X antigen polypeptides in the sera of hepatitis B virus-infected patients. Virol 177: 367-371, 1990.
14. Feitelson, M. A., Clayton, M. M. and Blumberg, B. S. X antigen/antibody markers in hepadavirus infections. Presence and significance of hepadnavirus X-gene products(s) in serum. Gastroenterol 98: 1071-1078, 1990.
15. Chen H Y, Tang N H, Lin N, Chen Z X, Wang X Z. Hepatitis B virus X protein induces apoptosis and cell cycle deregulation through interfering with DNA repair and checkpoint responses. Hepatol Res 38:174-82, 2008.
16. Pollicino T, Terradillos O, Lecoeur H, Gougeon M L, Buendia M A. Pro-apoptotic effect of the hepatitis B virus X gene. Biomed Pharmacother 52:363-8, 1998.
17. Zhang X, Dong N, Yin L, Cai N, Ma H, You J, Zhang H, Wang H, He R, Ye L. Hepatitis B virus X protein upregulates survivin expression in hepatoma tissues. J Med Virol 77: 374-81, 2005.
18. Pan J, Clayton M, Feitelson M A. Hepatitis B virus X antigen promotes transforming growth factor-beta1 (TGF-beta1) activity by up-regulation of TGF-beta1 and down-regulation of alpha2-macroglobulin. J Gen Virol 85(Pt 2): 275-82, 2004.
19. Lee S W, Lee Y M, Bae S K, Murakami S, Yun Y, Kim K W. Human hepatitis B virus X protein is a possible mediator of hypoxia-induced angiogenesis in hepatocarcinogenesis. Biochem Biophys Res Commun 268: 456-61, 2000.
20. Keasler V V, Lerat H, Madden C R, Finegold M J, McGarvey M J, Mohammed E M, Forbes S J, Lemon S M, Hadsell D L, Grona S J, Hollinger F B, Slagle B L. Increased liver pathology in hepatitis C virus transgenic mice expressing the hepatitis B virus X protein. Virol 347:466-75, 2006.
21. Chan D W, Ng I O. Knock-down of hepatitis B virus X protein reduces the tumorigenicity of hepatocellular carcinoma cells. J Pathol 208: 372-80, 2006.
22. Kayhan H, Karatayli E, Turkyilmaz A R, Sahin F, Yurdaydin C, Bozdayi A M. Inhibition of hepatitis B virus replication by shRNAs in stably HBV expressed HEPG2 2.2.15 cell lines. Arch Virol 152:871-9, 2007.
23. Ren J L, Pan J S, Cheng T, Dong J, Lu Y P, Huang S J, Shi H X, Wang L, Lian Y M. RNA interference inhibits hepatitis B virus gene expression and replication in HepG2-N10 cells. Chin J Dig Dis 7: 230-6, 2006.
24. Zhao Z F, Yang H, Han D W, Zhao L F, Zhang G Y, Zhang Y, Liu M S. Inhibition of hepatitis B virus expression and replication by RNA interference in HepG2.2.15. World J Gastroenterol 12: 6046-9, 2006.
25. Li H, Chi C Y, Lee S, Andrisani O M. The mitogenic function of hepatitis B virus X protein resides within amino acids 51 to 140 and is modulated by N- and C-terminal regulatory regions. J Virol. 2006 November; 80(21):10554-64.
26. Murakami S, Cheong J H, and Kaneko S. Human Hepatitis Virus X gene encodes a regulatory domain that represses transactivation of X protein. J Biol Chem 269:15118-15123, 1994.
27. Shao Y, Aplin A E. Akt3-mediated resistance to apoptosis in B-RAF-targeted melanoma cells. Cancer Res 70: 6670-81, 2010.
28. Wellbrock C, Hurlstone A. BRAF as therapeutic target in melanoma. Biochem Pharmacol 80: 561-7, 2010.
29. Tanami H, Imoto I, Hirasawa A, Yuki Y, Sonoda I, Inoue J, Yasui K, Misawa-Furihata A, Kawakami Y, Inazawa J. Involvement of overexpressed wild-type BRAF in the growth of malignant melanoma cell lines. Oncogene 23: 8796-804, 2004.
30. Bollag et al. Clinical efficacy of a RAF inhibitor needs broad target blockade in BRAF mutant melanoma. Nature 467: 596-9, 2010.
31. Rushworth L K, Hindley A D, O'Neill E, Kolch W. Regulation and role of Raf-1/B-Raf heterodimerization. Mol Cell Biol 26: 2262-72, 2006.
32. Yokoyama K, Nio N, Kikuchi Y. Properties and applications of microbial transglutaminase. Appl Microbiol Biotechnol. 2004 May; 64(4):447-54.

1. An isolated peptide consisting of a first segment having an amino acid sequence selected from the group consisting of:
(SEQ ID NO: 1)
LCLRP;
(SEQ ID NO: 2)
LCLRPVG;
(SEQ ID NO: 90)
LCLRPVGAESRGRPV;
(SEQ ID NO: 6)
LCLRPVGAESRGRPVSGPFG;
(SEQ ID NO: 71)
LCLRPVGAESRGRPVSGPF;
(SEQ ID NO: 70)
LCLRPVGAESRGRPVSGP;
(SEQ ID NO: 69)
LCLRPVGAESRGRPVSG;
(SEQ ID NO: 68)
LCLRPVGAESRGRPVS;
(SEQ ID NO: 67)
LCLRPVGAESRGRPV;
(SEQ ID NO: 66)
LCLRPVGAESRGRP;
(SEQ ID NO: 65)
LCLRPVGAESRG;
(SEQ ID NO: 64)
LCLRPVGAER;
and
(SEQ ID NO: 3)
MAARLCCQLDPARDVLCLRP;
or a functionally equivalent variant thereof;
and, optionally, a second segment connected to the first segment and comprising one or more heterologous amino acids such that the isolated peptide is a fusion peptide.

2. An isolated peptide comprising the amino acid sequence MAARLCCQ (SEQ ID NO: 7), or a functionally equivalent variant thereof, excluding peptides consisting of the full length sequence of the Hepatitis B virus X-protein and a peptide consisting of the amino acid sequence MAARVCCQL.

3. The isolated peptide of claim 2, further comprising at its C-terminus one or more consecutive amino acids of amino acids 9 to 35 of a native X-protein.

4. The isolated peptide of claim 3, wherein the peptide comprises the amino acid sequence MAARLCCQLDPARDV (SEQ ID NO: 8), or a functionally equivalent variant thereof.

5. The isolated peptide of claim 2, wherein the amino acid sequence is MAARLCCQ (SEQ ID NO: 7) or MAARLCCQLDPARDV (SEQ ID NO: 8).

6. An isolated nucleic acid encoding the peptide or functionally equivalent variant of claim 1 or 2 or a vector comprising said nucleic acid.

7. A construct comprising a peptide comprising the amino acid sequence LCLRP (SEQ ID NO: 1) or the amino acid sequence MAARLCCQ (SEQ ID NO: 7), or a functionally equivalent variant of any one thereof and a compound desired to be delivered to a cell.

8. The construct of claim 7, wherein the compound is a nucleic acid, a peptide nucleic acid, a polypeptide, a carbohydrate, a peptidomimetic, a small molecule inhibitor, a proteoglycan, lipid, a lipoprotein, a glycolipid, a natural product, or a glycomimetic.

9. The construct of claim 8, wherein the compound is a peptide comprising the amino acid sequence LVRSPAPCKFFTSA (SEQ ID NO: 9) or a functionally equivalent variant thereof, KLVRSPAPCKFFTSA (SEQ ID NO: 10) or a functionally equivalent variant thereof, HKLVRSPAPCKFFTSA (SEQ ID NO: 11) or a functionally equivalent variant thereof, RHKLVRSPAPCKFFTSA (SEQ ID NO: 12) or a functionally equivalent variant thereof, or GGCRHKLVRSPAPCKFFTSA (SEQ ID NO: 91), or a functionally equivalent variant thereof.

10. The construct of claim 8, wherein the compound is MLAPYIPM (SEQ ID NO: 13) or a functionally equivalent variant thereof.

11. A nucleic acid encoding the construct of claim 7 or a vector comprising said nucleic acid.

12. A method for increasing the cell membrane permeability of a compound, the method comprising connecting a peptide comprising the amino acid sequence LCLRP (SEQ ID NO: 1), MAARLCCQ (SEQ ID NO: 7), or a functionally equivalent variant thereof, to the compound.

13. A method for delivering a compound into a cell, the method comprising contacting the cell or a composition comprising the cell with a construct that includes the compound and a peptide comprising the amino acid sequence LCLRP (SEQ ID NO: 1) or the amino acid sequence MAARLCCQ (SEQ ID NO: 7), or a functionally equivalent variant of any one thereof; or a nucleic acid encoding the construct.

14. The method of claim 13, wherein the contacting step is accomplished by administering the construct or the nucleic acid encoding the construct to a subject.

15. A method for targeting delivery of a compound to adherent cells in a mixed population of adherent and non-adherent cells, the method comprising contacting the construct of claim 7 or a nucleic acid encoding the construct with a mixed population of cells or a composition comprising a mixed population of cells.

16. The method of claim 15, wherein the method is for targeting delivery of a compound to adherent cells in a subject, the method comprising administering the construct or a nucleic acid encoding the construct to the subject.

17. A host cell comprising the isolated nucleic acid of claim 6 or 11.


SEQUENCE LISTING (145 sequences)

SEQ ID NO: 1 - protein, 5 aa, Artificial sequence
  Synthetic polypeptide
LCLRP

SEQ ID NO: 2 - protein, 7 aa, Artificial sequence
  Synthetic polypeptide
LCLRPVG

SEQ ID NO: 3 - protein, 20 aa, Artificial sequence
  Synthetic polypeptide
MAARLCCQLDPARDVLCLRP

SEQ ID NO: 4 - protein, 9 aa, Artificial sequence
  Synthetic polypeptide
LCLRPVGAE

SEQ ID NO: 5 - protein, 11 aa, Artificial sequence
  Synthetic polypeptide
LCLRPVGAESR

SEQ ID NO: 6 - protein, 20 aa, Artificial sequence
  Synthetic polypeptide
LCLRPVGAESRGRPVSGPFG

SEQ ID NO: 7 - protein, 8 aa, Artificial sequence
  Synthetic polypeptide
MAARLCCQ

SEQ ID NO: 8 - protein, 15 aa, Artificial sequence
  Synthetic polypeptide
MAARLCCQLDPARDV

SEQ ID NO: 9 - protein, 14 aa, Artificial sequence
  Synthetic polypeptide
LVRSPAPCKFFTSA

SEQ ID NO: 10 - protein, 15 aa, Artificial sequence
  Synthetic polypeptide
KLVRSPAPCKFFTSA

SEQ ID NO: 11 - protein, 16 aa, Artificial sequence
  Synthetic polypeptide
HKLVRSPAPCKFFTSA

SEQ ID NO: 12 - protein, 17 aa, Artificial sequence
  Synthetic polypeptide
RHKLVRSPAPCKFFTSA

SEQ ID NO: 13 - protein, 8 aa, Artificial sequence
  Synthetic polypeptide
MLAPYIPM

SEQ ID NO: 14 - protein, 15 aa, Artificial sequence
  Synthetic polypeptide
RRRRRRRRRYDRREY

SEQ ID NO: 15 - protein, 14 aa, Artificial sequence
  Synthetic polypeptide
LCLRPVGGYDRREY

SEQ ID NO: 16 - protein, 6 aa, Artificial sequence
  Synthetic polypeptide
YDRREY

SEQ ID NO: 17 - protein, 21 aa, Artificial sequence
  Synthetic polypeptide
LCLRPVGGGRRRQQQQQQRRR

SEQ ID NO: 18 - DNA, 18 bp, Artificial sequence
  Synthetic oligonucleotide
gagctgcacgctgccgtc

SEQ ID NO: 19 - protein, 49 aa, Artificial sequence
  Synthetic polypeptide
RRRRRRRRHKLVRSPAPCKFFTSAGGLNVTAPTPQQLQAFKNEVGVLRK

SEQ ID NO: 20 - protein, 28 aa, Artificial sequence
  Synthetic polypeptide
RRRRRRRRMAARLCCQLDPARDVLCLRP

SEQ ID NO: 21 - protein, 35 aa, Artificial sequence
  Synthetic polypeptide
MAARVCCQLDPARDVLCLRPVGAESRGRPVSGPFG

SEQ ID NO: 22 - protein, 35 aa, Artificial sequence
  Synthetic polypeptide
MAARMCCQLDPARDVLCLRPVGAESRGRPVSGPFG

SEQ ID NO: 23 - protein, 35 aa, Artificial sequence
  Synthetic polypeptide
MAARLCCQLDPARDVLCLRPVGAESRGRPFSGPFG

SEQ ID NO: 24 - protein, 35 aa, Artificial sequence
  Synthetic polypeptide
MAARVCCQLDPARDVLCLRPVGAESRGRPVSRPFG

SEQ ID NO: 25 - protein, 35 aa, Artificial sequence
  Synthetic polypeptide
MAARLCCQLDPARDVLCLRPVGAESRGRPFSGPLG

SEQ ID NO: 26 - protein, 35 aa, Artificial sequence; identical to SEQ ID NO: 25
  Synthetic polypeptide

SEQ ID NO: 27 - protein, 35 aa, Artificial sequence
  Synthetic polypeptide
MAARLCCQLDPTRDVLCLRPVGAESRGRPVSGPLG

SEQ ID NO: 28 - protein, 35 aa, Artificial sequence; identical to SEQ ID NO: 25
  Synthetic polypeptide

SEQ ID NO: 29 - protein, 35 aa, Artificial sequence; identical to SEQ ID NO: 25
  Synthetic polypeptide

SEQ ID NO: 30 - protein, 35 aa, Artificial sequence
  Synthetic polypeptide
MAARLCCQLDPSRDVLCLRPVGAESRGRPLSGPLG

SEQ ID NO: 31 - protein, 35 aa, Artificial sequence
  Synthetic polypeptide
MAARLCCQLDPARDVLCLRPVGAESRGRPVSGSLG

SEQ ID NO: 32 - protein, 35 aa, Artificial sequence
  Synthetic polypeptide
MAARLCCQLDPARDVLCLRPVGAESRGRPLPGPLG

SEQ ID NO: 33 - protein, 35 aa, Artificial sequence; identical to SEQ ID NO: 32
  Synthetic polypeptide

SEQ ID NO: 34 - protein, 35 aa, Artificial sequence; identical to SEQ ID NO: 32
  Synthetic polypeptide

SEQ ID NO: 35 - protein, 35 aa, Artificial sequence; identical to SEQ ID NO: 32
  Synthetic polypeptide

SEQ ID NO: 36 - protein, 35 aa, Artificial sequence
  Synthetic polypeptide
MATRLCCQLDPSRDVLCLRPVGAESRGRPLSGPLG

SEQ ID NO: 37 - protein, 35 aa, Artificial sequence; identical to SEQ ID NO: 32
  Synthetic polypeptide

SEQ ID NO: 38 - protein, 35 aa, Artificial sequence
  Synthetic polypeptide
MAARLCCQLDPARDVLCLRPVGAESRGRPFSGSLG

SEQ ID NO: 39 - protein, 35 aa, Artificial sequence
  Synthetic polypeptide
MAARLCCQLDPARDVLCLRPVGAESRGRPFPGPLG

SEQ ID NO: 40 - protein, 35 aa, Artificial sequence
  Synthetic polypeptide
MAARMCCQLDPARDVLCLRPVGAESRGRPLPGPLG

SEQ ID NO: 41 - protein, 35 aa, Artificial sequence
  Synthetic polypeptide
MAARVCCQLDPARDVLCLRPVGAESRGRPLPGPLG

SEQ ID NO: 42 - protein, 35 aa, Artificial sequence
  Synthetic polypeptide
MAARLYCQLDPSRDVLCLRPVGAESRGRPLSGPLG

SEQ ID NO: 43 - protein, 35 aa, Artificial sequence; identical to SEQ ID NO: 42
  Synthetic polypeptide

SEQ ID NO: 44 - protein, 35 aa, Artificial sequence
  Synthetic polypeptide
MAARLCCQLDPSRDVLCLRPVSAESSGRPLPGPFG

SEQ ID NO: 45 - protein, 35 aa, Artificial sequence
  Synthetic polypeptide
MAARLCCQLDTARDVLCLRPVGAESRGRPLPGPLG

SEQ ID NO: 46 - protein, 35 aa, Artificial sequence
  Synthetic polypeptide
MAARMCCKLDPARDVLCLRPIGAESRGRPLPGPLG

SEQ ID NO: 47 - protein, 35 aa, Artificial sequence
  Synthetic polypeptide
MAARLCCQLDPARDVLCLRPVGAESCGRPVSGSLG

SEQ ID NO: 48 - protein, 35 aa, Artificial sequence
  Synthetic polypeptide
MAARLCCQLDPARDVLCLRPVGAESRGRSLSGSLG

SEQ ID NO: 49 - protein, 35 aa, Artificial sequence
  Synthetic polypeptide
MAARLCCQLDTARDVLCLRPVGAESRGRPFSGSVG

SEQ ID NO: 50 - protein, 35 aa, Artificial sequence
  Synthetic polypeptide
MAARLCCQLDPARDVLCLRPVSAESCGRPVSGSLG

SEQ ID NO: 51 - protein, 35 aa, Artificial sequence; identical to SEQ ID NO: 50
  Synthetic polypeptide

SEQ ID NO: 52 - protein, 35 aa, Artificial sequence
  Synthetic polypeptide
MAARLYCQLDSSRDVLCLRPVGAESRGRPFSGPLG

SEQ ID NO: 53 - protein, 35 aa, Artificial sequence
  Synthetic polypeptide
MAARLYCQLDSSRDVLCLRPVGAESRGRPLAGPLG

SEQ ID NO: 54 - protein, 35 aa, Artificial sequence
  Synthetic polypeptide
MAARLCCQLDPARDVLCLRPVGAESCGRPLSWSLG

SEQ ID NO: 55 - protein, 35 aa, Artificial sequence
  Synthetic polypeptide
MAARLCCQLDPARDVLCLRPVSAESCGRSVSGSLG

SEQ ID NO: 56 - protein, 34 aa, Artificial sequence
  Synthetic polypeptide
MAARLCCQLDTSRDVLCLRPVGAESCGRPFSGPL

SEQ ID NO: 57 - protein, 35 aa, Artificial sequence
  Synthetic polypeptide
MAARLCCQLDPARDVLCLRPVGAESSGRTLPGSLG

SEQ ID NO: 58 - protein, 31 aa, Artificial sequence
  Synthetic polypeptide
MAARLCCQLDPARDVLCLRPVGAESCGRPLS

SEQ ID NO: 59 - protein, 31 aa, Artificial sequence; identical to SEQ ID NO: 58
  Synthetic polypeptide

SEQ ID NO: 60 - protein, 29 aa, Artificial sequence
  Synthetic polypeptide
MAARMCCQLDPSQDVLCLRPVGAESRGRP

SEQ ID NO: 61 - protein, 32 aa, Artificial sequence
  Synthetic polypeptide
MAARLCCQLDPARDVLCLRPVGAEPCRRPVSG

SEQ ID NO: 62 - protein, 35 aa, Artificial sequence
  Synthetic polypeptide
MAARLYCQLDSSRNVLCLRPVGAESCGRPLSGPVG

SEQ ID NO: 63 - protein, 8 aa, Artificial sequence
  Synthetic polypeptide
LCLRPVGA

SEQ ID NO: 64 - protein, 10 aa, Artificial sequence
  Synthetic polypeptide
LCLRPVGAER

SEQ ID NO: 65 - protein, 12 aa, Artificial sequence
  Synthetic polypeptide
LCLRPVGAESRG

SEQ ID NO: 66 - protein, 14 aa, Artificial sequence
  Synthetic polypeptide
LCLRPVGAESRGRP

SEQ ID NO: 67 - protein, 15 aa, Artificial sequence
  Synthetic polypeptide
LCLRPVGAESRGRPV

SEQ ID NO: 68 - protein, 16 aa, Artificial sequence
  Synthetic polypeptide
LCLRPVGAESRGRPVS

SEQ ID NO: 69 - protein, 17 aa, Artificial sequence
  Synthetic polypeptide
LCLRPVGAESRGRPVSG

SEQ ID NO: 70 - protein, 18 aa, Artificial sequence
  Synthetic polypeptide
LCLRPVGAESRGRPVSGP

SEQ ID NO: 71 - protein, 19 aa, Artificial sequence
  Synthetic polypeptide
LCLRPVGAESRGRPVSGPF

SEQ ID NO: 72 - DNA, 15 bp, Artificial sequence
  Synthetic oligonucleotide
ctttgtctacgtccc

SEQ ID NO: 73 - DNA, 21 bp, Artificial sequence
  Synthetic oligonucleotide
ctttgtctacgtcccgtcggc

SEQ ID NO: 74 - DNA, 27 bp, Artificial sequence
  Synthetic oligonucleotide
ctttgtctacgtcccgtcggcgctgaa

SEQ ID NO: 75 - DNA, 33 bp, Artificial sequence
  Synthetic oligonucleotide
ctttgtctacgtcccgtcggcgctgaatcccgc

SEQ ID NO: 76 - DNA, 60 bp, Artificial sequence
  Synthetic oligonucleotide
ctttgtctacgtcccgtcggcgctgaatcccgcggacgacccgtctcggggccgtttggg

SEQ ID NO: 77 - DNA, 60 bp, Artificial sequence
  Synthetic oligonucleotide
atggctgctaggctgtgctgccaactggatcctgcgcgggacgtcctttgtctacgtccc

SEQ ID NO: 78 - protein, 38 aa, Artificial sequence
  Synthetic polypeptide
RRRRRRRRMLAPYIPMGGLCLRPVGAESRGRPVSGPFG

SEQ ID NO: 79 - protein, 37 aa, Artificial sequence
  Synthetic polypeptide
RRRRRRRRLCLRPVGAESRGRPVSGPFGGMLAPYIPM

SEQ ID NO: 80 - protein, 46 aa, Artificial sequence
  Synthetic polypeptide
RRRRRRRRHKLVRSPAPCKFFTSAGGLCLRPVGAESRGRPVSGPFG

SEQ ID NO: 81 - protein, 47 aa, Artificial sequence
  Synthetic polypeptide
RRRRRRRRLCLRPVGAESRGRPVSGPFGGCRHKLVRSPAPCKFFTSA

SEQ ID NO: 82 - protein, 47 aa, Artificial sequence; identical to SEQ ID NO: 81
  Synthetic polypeptide

SEQ ID NO: 83 - protein, 37 aa, Artificial sequence; identical to SEQ ID NO: 79
  Synthetic polypeptide

SEQ ID NO: 84 - protein, 20 aa, Artificial sequence
  Synthetic polypeptide
VGAESRGRPVSGPFGPLSSP

SEQ ID NO: 85 - protein, 20 aa, Artificial sequence
  Synthetic polypeptide
FGPLSSPAFSVPADHGAHLS

SEQ ID NO: 86 - protein, 50 aa, Artificial sequence
  Synthetic polypeptide
MAARLCCQLDPARDVLCLRPVGAESRGRPVSGPFGPLSSPAFSVPADHGA

SEQ ID NO: 87 - DNA, 452 bp, Artificial sequence
  Synthetic oligonucleotide
gggaagcttggtaccatggctgctaggctgtgctgccaactggatcctgcgcgggacgtcctttgtctacgtcccgtcggcgctgaatcccgcggacgacccgtctcggggccgtttgggcctctatcgtcccctgctttctccgttccggccgaccacggggcgcacctctctttacgcggtctccccgtctgtgccttctcatctgccggaccgtgtgcacttcgcttcacctctgcacgtcgcatggagaccaccgtgaacggccgccggggcttgcccaaggtcttgcataagaggactcttggactctcagcaatgtcaacgaccgaccctgaggcatacttcaaagactgtgtgttcacatactggagggagttgggggcagagggtaggttaaaggtctttgtactaggaggctgtaggcataaattgtagctctagtctagagc

SEQ ID NO: 88 - DNA, 452 bp, Artificial sequence
  Synthetic oligonucleotide
gctctagactagagctacaatttatgcctacagcctcctagtacaaagacctttaacctaccctctgcccccaactccctccagtatgtgaacacacagtctttgaagtatgcctcagggtcggtcgttgacattgctgagagtccaagagtcctcttatgcaagaccttgggcaagccccggcggccgttcacggtggtctccatgcgacgtgcagaggtgaagcgaagtgcacacggtccggcagatgagaaggcacagacggggagaccgcgtaaagagaggtgcgccccgtggtcggccggaacggagaaagcaggggacgatagaggcccaaacggccccgagacgggtcgtccgcgggattcagcgccgacgggacgtagacaaaggacgtcccgcgcaggatccagttggcagcacagcctagcagccatggtaccaagcttccc

SEQ ID NO: 89 - protein, 23 aa, Artificial sequence
  Synthetic polypeptide
LNVTAPTPQQLQAFKNEVGVLRK

SEQ ID NO: 90 - protein, 15 aa, Artificial sequence
  Synthetic polypeptide
LCLRPVGAESRGRPV

SEQ ID NO: 91 - protein, 20 aa, Artificial sequence
  Synthetic polypeptide
GGCRHKLVRSPAPCKFFTSA

SEQ ID NO: 92 - protein, 23 aa, Artificial sequence
  Synthetic polypeptide
TTHNFVRKTFFTLAFCDFCRKLL

SEQ ID NO: 93 - protein, 23 aa, Artificial sequence
  Synthetic polypeptide
SLPGSLTNVKALQKSPGPQRERK

SEQ ID NO: 94 - protein, 8 aa, Artificial sequence
  Synthetic polypeptide
RKTFLKLA

SEQ ID NO: 95 - protein, 7 aa, Artificial sequence
  Synthetic polypeptide
CCAVFRL

SEQ ID NO: 96 - protein, 44 aa, Artificial sequence
  Synthetic polypeptide
MPNLWFLLFLGLVAAMQLLLLLFLLLFFLVYWDHFECSCTGLPF

SEQ ID NO: 97 - protein, 5 aa, Artificial sequence
  Synthetic polypeptide
YIGSR

SEQ ID NO: 98 - protein, 25 aa, Artificial sequence
  Synthetic polypeptide
NLEASPHISDPHEARGTRPGLYLYS

SEQ ID NO: 99 - protein, 11 aa, Artificial sequence
  Synthetic polypeptide
GGCRHNFFTSA

SEQ ID NO: 100 - protein, 20 aa, Artificial sequence
  Synthetic polypeptide
GGCRHKLVCSPAPCNFFTSA

SEQ ID NO: 101 - protein, 20 aa, Artificial sequence
  Synthetic polypeptide
GGCRHKLVCSPAPCNFFTSA

SEQ ID NO: 102 - protein, 20 aa, Artificial sequence
  Synthetic polypeptide
GGCRHKLVCSPAPCNFFTSA

SEQ ID NO: 103 - protein, 20 aa, Artificial sequence
  Synthetic polypeptide
GGCRHKLVCAPAPCNFFTSA

SEQ ID NO: 104 - protein, 20 aa, Artificial sequence
  Synthetic polypeptide
GGCRHKLVCSPAPCNFFHLC

SEQ ID NO: 105 - protein, 20 aa, Artificial sequence
  Synthetic polypeptide
GGCRHKLVCAPAPCNFFTSA

SEQ ID NO: 106 - protein, 20 aa, Artificial sequence
  Synthetic polypeptide
GGCRHKLVCAPAPCNFFTSA

SEQ ID NO: 107 - protein, 20 aa, Artificial sequence
  Synthetic polypeptide
GGCRHKLVCSPAPCNFFTSA

SEQ ID NO: 108 - protein, 20 aa, Artificial sequence
  Synthetic polypeptide
GGCRHKLVCAPAPCNFFTSA

SEQ ID NO: 109 - protein, 20 aa, Artificial sequence
  Synthetic polypeptide
GGCRHKLVCSPAPCNFFTSA

SEQ ID NO: 110 - protein, 20 aa, Artificial sequence
  Synthetic polypeptide
GGCRHKLVCAPAPCNFFTSA

SEQ ID NO: 111 - protein, 20 aa, Artificial sequence
  Synthetic polypeptide
GGCRHKLVCVPAPCNFFTSA

SEQ ID NO: 112 - protein, 20 aa, Artificial sequence
  Synthetic polypeptide
GGCRHKLVCSPAPCNFFTSA

SEQ ID NO: 113 - protein, 20 aa, Artificial sequence
  Synthetic polypeptide
GGCRHKLVCSPAPCNFFTSA

SEQ ID NO: 114 - protein, 20 aa, Artificial sequence
  Synthetic polypeptide
GGCRHKLVCSPAPCNFFTSA

SEQ ID NO: 115 - protein, 20 aa, Artificial sequence
  Synthetic polypeptide
GGCRHKLVCSPAPCNFFTSA

SEQ ID NO: 116 - protein, 20 aa, Artificial sequence
  Synthetic polypeptide
GGCRHKLVCAPAPCNFFTSA

SEQ ID NO: 117 - protein, 20 aa, Artificial sequence
  Synthetic polypeptide
GGCRHKLVCPPAPCNFFTSA

SEQ ID NO: 118 - protein, 20 aa, Artificial sequence
  Synthetic polypeptide
GGCRHKLVCAPAPCNFFTSA

SEQ ID NO: 119 - protein, 20 aa, Artificial sequence
  Synthetic polypeptide
GGCRHKLVCSPAPCNFFTSA

SEQ ID NO: 120 - protein, 20 aa, Artificial sequence
  Synthetic polypeptide
GGCRHKLVCSPAPCNFFTSA

SEQ ID NO: 121 - protein, 20 aa, Artificial sequence
  Synthetic polypeptide
GGCRHKLVCSPAPCNFFTSA

SEQ ID NO: 122 - protein, 20 aa, Artificial sequence
  Synthetic polypeptide
GGCRHKLVCAPAPCNFFTSA

SEQ ID NO: 123 - protein, 20 aa, Artificial sequence
  Synthetic polypeptide
GGCRHKLVCAPAPCNFFTSA

SEQ ID NO: 124 - protein, 20 aa, Artificial sequence
  Synthetic polypeptide
GGCRHKLVCAPAPCNFFTSA

SEQ ID NO: 125 - protein, 20 aa, Artificial sequence
  Synthetic polypeptide
GGCRHKLVCSPAPCNFFTSA

SEQ ID NO: 126 - protein, 20 aa, Artificial sequence
  Synthetic polypeptide
GGCRHKLVCSPAPCNFFTSA

SEQ ID NO: 127 - protein, 20 aa, Artificial sequence
  Synthetic polypeptide
GGCRHKLVCVPAPCNFFTSA

SEQ ID NO: 128 - protein, 20 aa, Artificial sequence
  Synthetic polypeptide
GGCRHKLVCSPAPCNFFTSA

SEQ ID NO: 129 - protein, 20 aa, Artificial sequence
  Synthetic polypeptide
GGCRHKLVCSPAPCNFFTSA

SEQ ID NO: 130 - protein, 20 aa, Artificial sequence
  Synthetic polypeptide
GGCRHKLVCAPAPCNFFTSA

SEQ ID NO: 131 - protein, 20 aa, Artificial sequence
  Synthetic polypeptide
GGCRHKLVCAPAPCNFFTSA

SEQ ID NO: 132 - protein, 20 aa, Artificial sequence
  Synthetic polypeptide
GGCRHKLVCAPSSCNFFTSA

SEQ ID NO: 133 - protein, 20 aa, Artificial sequence
  Synthetic polypeptide
GGCRHKLVFAPSSCNFFTSA

SEQ ID NO: 134 - protein, 20 aa, Artificial sequence
  Synthetic polypeptide
GGCRHKLVCSPAPCNFFTSA

SEQ ID NO: 135 - protein, 20 aa, Artificial sequence
  Synthetic polypeptide
GGCRHKLVCAPAPCNFFTSA

SEQ ID NO: 136 - protein, 20 aa, Artificial sequence
  Synthetic polypeptide
GGCRHKLVCTPAPCNFFTSA

SEQ ID NO: 137 - protein, 20 aa, Artificial sequence
  Synthetic polypeptide
GGCRHKLVCSPAPCNFFTSA

SEQ ID NO: 138 - protein, 20 aa, Artificial sequence
  Synthetic polypeptide
GGCRHKLVCSPAPCNFFTSA

SEQ ID NO: 139 - protein, 20 aa, Artificial sequence
  Synthetic polypeptide
GGCRHKLVCSPAPCNFFTSA

SEQ ID NO: 140 - protein, 20 aa, Artificial sequence
  Synthetic polypeptide
GGCRHKLVCSPAPCNFFTSA

SEQ ID NO: 141 - protein, 20 aa, Artificial sequence
  Synthetic polypeptide
GGCRHKLVCAPAPCNFFTSA

SEQ ID NO: 142 - protein, 20 aa, Artificial sequence
  Synthetic polypeptide
GGCRHKLVCAPFSCNFFTSA

SEQ ID NO: 143 - protein, 4 aa, Artificial sequence
  Synthetic peptide
RRRR

SEQ ID NO: 144 - protein, 8 aa, Artificial sequence
  Synthetic peptide
RRRRRRRR

SEQ ID NO: 145 - protein, 9 aa, Artificial sequence
  Synthetic peptide
RRRRRRRRR